(19) United States
(12) Patent Application Publication
(10) Pub. No.: US 2016/0153039 A1
(43) Pub. Date: Jun. 2, 2016

(54) COMPOSITIONS AND METHODS FOR TARGETED NUCLEIC ACID SEQUENCE ENRICHMENT AND HIGH EFFICIENCY LIBRARY GENERATION

(71) Applicant: NuGEN Technologies, Inc., San Carlos, CA (US)

(72) Inventors: Doug Amorese, Los Altos, CA (US); Nurith Kurn, Palo Alto, CA (US); Jonathan Scolnick, San Francisco, CA (US); Alexandra Hui Wang, Mountain View, CA (US)

(21) Appl. No.: 14/836,936

(22) Filed: Aug. 26, 2015

Related U.S. Application Data
(60) Provisional application No. 62/042,240, filed on Aug. 26, 2014.
(60) Provisional application No. 62/188,337, filed on Jul. 2, 2015.

(51) Int. Cl. C12Q 1/68; C12P 19/34

(52) U.S. Cl. CPC C12Q 1/6874; C12P 19/34

(57) ABSTRACT

Provided herein are methods, compositions and kits for targeted nucleic acid sequence enrichment in a nucleic acid sample and for high efficiency nucleic acid library generation for next generation sequencing (NGS). The methods, compositions and kits provided herein can be useful for the production and capture of amplification-ready, target-specific and strand-specific regions of interest from nucleic acid samples containing complex DNA.

### CROSS-REFERENCE

This application claims priority to U.S. Provisional Application No. 62/042,240, filed Aug. 26, 2014 and U.S. Provisional Application No. 62/188,337, filed Jul. 2, 2015, which applications are incorporated herein by reference. This application is related to U.S. Provisional Application No. 61/591,241, filed Jan. 26, 2012; PCT Application No. PCT/US13/23278, filed Jan. 25, 2013, and U.S. patent application Ser. No. 13/750,768, which applications are incorporated herein by reference.

### BACKGROUND OF THE INVENTION

With the rapid development of next generation sequencing (NGS) technologies and platforms, whole genome sequencing is becoming increasingly feasible. Researchers are driven to generate increasing amounts of data to achieve greater understanding of variance and biological trends, and to generate data from smaller sample sizes to avoid averaging across multiple cells within a tissue.

Although the cost of whole genome sequencing is decreasing and the throughput of the NGS platforms is increasing, it is nonetheless often more practical and cost-effective to select genomic regions of interest for sequencing and analysis. Target enrichment is a commonly employed strategy in genomic DNA sequencing in which genomic regions of interest are selectively captured from a DNA sample before sequencing. Focused target enrichment is an important tool especially in the fields of study where sequencing of a large number of samples is necessary (e.g. population-based studies of disease markers or SNPs), making whole genome sequencing cost-prohibitive. Similarly, improvements have been made that enable DNA libraries to be made from nucleic acid from fewer number of cells, but these are bound by the limitations of the efficiency of ligation reactions.

Several approaches to target enrichment have been developed which vary from one another in terms of sensitivity, specificity, reproducibility, uniformity, cost and ease of use. The target enrichment methods commonly employed today can be divided into three major categories, each with its distinct advantages and disadvantages: 1) PCR-based methods; 2) capture-by-hybridization, e.g., on-array or in-solution hybrid capture; and 3) capture-by-circularization, e.g., molecular inversion probe-based methods.

The PCR-based methods employ highly parallel PCR amplification, where each target sequence in the sample has a corresponding pair of unique, sequence-specific primers. The simultaneous use of numerous primer pairs makes multiplex PCR impractical due to high level of non-specific amplification and primer-primer interactions. Recently developed microdroplet PCR technology (Tewhey et al., 2009) in which each amplification reaction is physically separated into an individual droplet removes the constraints of multiplex PCR relating to non-specific amplification and primer-primer interactions. However, microdroplet PCR and other improved PCR-based methods require special instrumentation or platforms, are limited in their throughput, and, as with conventional multiplex PCR, require a large number of individual primer pairs when enriching for a multitude of regions on interest, thus making target enrichment costly.

Hybrid capture methods are based on the selective hybridization of the target genomic regions to user-designed oligonucleotides. The hybridization can be to oligonucleotides immobilized on high or low density microarrays (on-array capture), or solution-phase hybridization to oligonucleotides modified with a ligand (e.g. biotin) which can subsequently be immobilized to a solid surface, such as a bead (in-solution capture). The hybrid capture methods require complex pools of costly long oligonucleotides and long periods (typically 48 hours) of hybridization for efficient capture. For on-array hybrid capture, expensive instrumentation and hardware is also required. Because of the relatively low efficiency of the hybridization reaction, large quantities of input DNA are needed.

The molecular inversion probe (MIP) based method relies on construction of numerous single-stranded linear oligonucleotide probes, consisting of a common linker flanked by target-specific sequences. Upon annealing to a target sequence, the probe gap region is filled via polymerization and ligation, resulting in a circularized probe. The circularized probes are then released and amplified using primers directed at the common linker region. One of the main disadvantages of the MIP-based target enrichment is its relatively low capture uniformity, meaning there is large variability in sequence coverage across the target regions. As with PCR and hybrid capture, the MIP-based method requires a large number of target-specific oligonucleotides, which can be costly.

There is a need for improved methods for selective target enrichment that allow for low-cost, high throughput capture of genomic regions of interest without specialized instrumentation. Additionally, there is also a need for high efficiency nucleic acid library generation. The methods of the invention described herein fulfills these needs.

### SUMMARY OF THE INVENTION

In one aspect, a method is provided for enriching for a nucleic acid sequence of interest in a sample comprising nucleic acids, the method comprising: a) annealing an oligonucleotide to the nucleic acid sequence of interest in a nucleic acid fragment, wherein the oligonucleotide comprises a 3′ portion complementary to the nucleic acid sequence of interest and a 5′ portion comprising a first adaptor sequence; b) extending the oligonucleotide annealed to the nucleic acid sequence of interest in the nucleic acid fragment with a polymerase, thereby generating an oligonucleotide extension product comprising sequence complementary to the nucleic acid sequence of interest and the first adaptor sequence at a first end; c) ligating a sequence complementary to a second adaptor sequence to the oligonucleotide extension product, thereby generating an oligonucleotide extension product comprising the first adaptor sequence at the first end, the sequence complementary to the nucleic acid sequence of interest, and a sequence complementary to the second adaptor sequence at a second end; and d) amplifying the nucleic acid sequence of interest using a first primer that anneals to the complement of the first adaptor sequence and a second primer that anneals to the complement of the second adaptor sequence to enrich for the nucleic acid sequence of interest, thereby generating an enriched nucleic acid sequence of interest. In some cases, the method further comprises, before step a), fragmenting a nucleic acid in the sample, thereby generating the nucleic acid fragment of step a) comprising the sequence of interest. In some cases, the nucleic acid fragment is double-stranded prior to step a). In some cases, the nucleic acid fragment comprises deoxyribonucleic acid (DNA). In some cases, the DNA is genomic DNA. In some cases, the DNA is cDNA. In some cases, the method further comprises denaturing the double-stranded nucleic acid fragment prior to step b), wherein the nucleic acid fragment comprising the nucleic acid sequence of interest in step b) is single-stranded prior to annealing to the oligonucleotide. In some cases, the 3′ portion of the oligonucleotide comprises a random sequence. In some cases, the 3′ portion of the oligonucleotide comprises a sequence designed to anneal to the nucleic acid sequence of interest. In some cases, ligating the sequence complementary to the second adaptor sequence comprises ligating the sequence complementary to the second adaptor sequence to a 3′ end of the oligonucleotide extension product. In some cases, the second adaptor sequence is within a strand of a double-stranded adaptor. In some cases, the first adaptor sequence and the second adaptor sequence are distinct. In some cases, the first adaptor sequence and/or the second adaptor sequence comprise a barcode sequence. In some cases, the first adaptor sequence and/or the second adaptor sequence comprise sequence that can anneal to a sequencing primer, or complement of a sequence that can anneal to a sequencing primer. In some cases, the polymerase is a DNA polymerase. In some cases, the method further comprises annealing a plurality of oligonucleotides to different nucleic acid sequences of interest in nucleic acid fragments, wherein each of the plurality of oligonucleotides comprises a 3′ portion complementary to a nucleic acid sequence of interest among the nucleic acid sequences of interest and a 5′ portion comprising a first adaptor sequence; extending the plurality of oligonucleotides annealed to the different nucleic acid sequences of interest in the nucleic acid fragments with a polymerase, thereby generating a plurality of oligonucleotide extension products comprising sequence complementary to the nucleic acid sequences of interest and the first adaptor sequence at a first end; ligating a sequence complementary to a second adaptor sequence to the plurality of oligonucleotide extension products, thereby generating a plurality of oligonucleotide extension products comprising the first adaptor sequence at the first end, the sequence complementary to the nucleic acid sequence of interest, and a sequence complementary to the second adaptor sequence at a second end; and amplifying the nucleic acid sequences of interest using a first primer that anneals to the complement of the first adaptor sequence and a second primer that anneals to the complement of the second adaptor sequence to enrich for the nucleic acid sequences of interest, thereby generating enriched nucleic acid sequences of interest.

In some cases, the amplifying of step d) comprises a polymerase chain reaction. In some cases, the method further comprises enriching a plurality of nucleic acid sequences of interest using the steps of the method. In some cases, the method further comprises sequencing the plurality of enriched nucleic acid sequences of interest. In some cases, the nucleic acid sequence of interest comprises a gene. In some cases, the gene is a cancer gene. In some cases, the method further comprises sequencing the nucleic acid sequence of interest. In some cases, the sequencing comprises use of a reversible-dye terminator.

In another aspect, a method is provided for enriching for a nucleic acid sequence of interest in a sample comprising nucleic acids, the method comprising: a) combining a transposome comprising a transposase and a transposon sequence with a nucleic acid in the sample, thereby generating a nucleic acid fragment comprising the nucleic acid sequence of interest and a transposon sequence appended to a first end of the nucleic acid fragment; b) annealing an oligonucleotide to the nucleic acid fragment comprising the transposon sequence at the first end, wherein the oligonucleotide comprises a 3′ portion that is complementary to the transposon sequence and a 5′ portion comprising a first adaptor sequence; c) extending the first oligonucleotide annealed to the transposon sequence with a polymerase, thereby generating a first oligonucleotide extension product comprising sequence complementary to the nucleic acid fragment and a first adaptor sequence at the first end; d) annealing a second oligonucleotide to a sequence complementary to the nucleic acid sequence of interest in the nucleic acid fragment comprising the first adaptor sequence at the first end, wherein the second oligonucleotide comprises a 3′ portion that anneals to the complement of the nucleic acid sequence of interest and a 5′ portion comprising a second adaptor sequence; e) extending the second oligonucleotide annealed to the complement of the nucleic acid sequence of interest in the nucleic acid fragment comprising the first adaptor sequence at the first end with a polymerase, thereby generating a second oligonucleotide extension product comprising sequence complementary to the first adaptor sequence at the first end, the nucleic acid sequence of interest, and the second adaptor sequence at the second end; and f) amplifying the nucleic acid sequence of interest using a first primer that anneals to the complement of the first adaptor sequence and a second primer that anneals to the complement of the second adaptor sequence to enrich for the nucleic acid sequence of interest. In some cases, the transposase is a Tn5 transposase. In some cases, the nucleic acid fragment of step a) is double stranded. In some cases, the method further comprises denaturing the double stranded nucleic acid fragment prior to step b), wherein the nucleic acid fragment in step b) is single-stranded prior to annealing to the oligonucleotide. In some cases, the first oligonucleotide extension product is annealed to the nucleic acid fragment after the extension step c). In some cases, the method further comprises denaturing the first oligonucleotide extension product from the nucleic acid fragment prior to step d), whereby the first oligonucleotide extension product in step d) is single stranded. In some cases, the 3′ portion of the second oligonucleotide comprises a random sequence. In some cases, the 3′ portion of the second oligonucleotide comprises a sequence designed to anneal to the nucleic acid sequence of interest. In some cases, the first adaptor sequence and the second adaptor sequence are distinct. In some cases, the first adaptor sequence and/or the second adaptor sequence comprise a barcode sequence. In some cases, the first adaptor sequence and/or the second adaptor sequence comprise sequence designed to anneal to a sequencing primer. In some cases, the polymerase is a DNA polymerase. In some cases, the amplification of step f) comprises polymerase chain reaction. In some cases, the annealing step d) comprises annealing a plurality of second oligonucleotides to a plurality of nucleic acid fragments, wherein the plurality of second oligonucleotides comprise a 3′ portion that anneals to the complement of a different nucleic acid sequence of interest in the nucleic acid fragments and a 5′ portion comprising the second adaptor sequence. In some cases, the method further comprises using steps a)-c) to produce a plurality of first oligonucleotide extension products comprising sequence complementary to nucleic acid fragments and a first adaptor sequence at the first end; annealing each of a plurality of second oligonucleotides to a sequence complementary to a nucleic acid sequence of interest in the nucleic acid fragments comprising the first adaptor sequence at the first end, wherein the plurality of second oligonucleotides comprise a 3′ portion that anneals to a complement of a nucleic acid sequence of interest and a 5′ portion comprising a second adaptor sequence; extending the plurality of second oligonucleotides annealed to a complement of a nucleic acid sequence of interest in a nucleic acid fragment comprising the first adaptor sequence at the first end with a polymerase, thereby generating a plurality of second oligonucleotide extension products comprising sequence complementary to the first adaptor sequence at the first end, a nucleic acid sequence of interest, and the second adaptor sequence at the second end; and amplifying the nucleic acid sequences of interest using a first primer that anneals to the complement of the first adaptor sequence and a second primer that anneals to the complement of the second adaptor sequence to enrich for the nucleic acid sequence of interest.

In some cases, the method further comprises enriching a plurality of nucleic acid sequences of interest using the steps of the method. In some cases, the method further comprises sequencing a plurality of enriched nucleic acid sequences of interest. In some cases, the sequencing comprises use of a reversible-dye terminator.

In another aspect, a method is provided for enriching for a nucleic acid sequence of interest in a sample comprising nucleic acids, the method comprising: a) combining a transposome comprising a transposase and a transposon sequence with a nucleic acid in the sample, thereby generating a nucleic acid fragment comprising the nucleic acid sequence of interest and a transposon sequence appended to a first end of the nucleic acid fragment; b) annealing an oligonucleotide to the nucleic acid fragment comprising the transposon sequence at the first end, wherein the oligonucleotide comprises a 3′ portion that is complementary to the nucleic acid sequence of interest in the nucleic acid fragment and a 5′ portion comprising a first adaptor sequence; c) extending the oligonucleotide annealed to the nucleic acid sequence of interest with a polymerase, thereby generating an oligonucleotide extension product comprising sequence complementary to the transposon sequence at a first end, sequence complementary to the nucleic acid sequence of interest, and a first adaptor sequence at a second end; and d) amplifying the nucleic acid sequence of interest using a first primer that anneals to the complement of the transposon sequence and a second primer that anneals to the complement of the first adaptor sequence to enrich for the nucleic acid sequence of interest. In some cases, the transposase is a Tn5 transposase. In some cases, the nucleic acid fragment of step a) is double stranded. In some cases, the method further comprises denaturing the double stranded nucleic acid fragment prior to step b), wherein the nucleic acid fragment in step b) is single-stranded prior to annealing to the oligonucleotide. In some cases, the oligonucleotide extension product is annealed to the nucleic acid fragment after the extension of step c). In some cases, the method further comprises denaturing the oligonucleotide extension product from the nucleic acid fragment prior to step d), whereby the oligonucleotide extension product in step d) is single stranded. In some cases, the first adaptor sequence comprises a barcode sequence. In some cases, the first adaptor sequence comprises sequence designed to anneal to a sequencing primer. In some cases, the polymerase is a DNA polymerase. In some cases, the method further comprises generating a plurality of nucleic acid fragments comprising a nucleic acid sequence of interest and a transposon sequence appended to a first end of the nucleic acid fragment; annealing a plurality of oligonucleotides to the nucleic acid fragments comprising the transposon sequence at the first end, wherein each of the plurality of oligonucleotides comprises a 3′ portion that is complementary to the nucleic acid sequence of interest in a nucleic acid fragment and a 5′ portion comprising a first adaptor sequence; extending the plurality of oligonucleotides annealed to the nucleic acid sequences of interest with a polymerase, thereby generating a plurality of oligonucleotide extension products comprising sequence complementary to the transposon sequence at a first end, sequence complementary to the nucleic acid sequence of interest, and a first adaptor sequence at a second end; and amplifying the nucleic acid sequences of interest using a first primer that anneals to the complement of the transposon sequence and a second primer that anneals to the complement of the first adaptor sequence to enrich for the nucleic acid sequences of interest.

In some cases, the amplification of step f) comprises polymerase chain reaction. In some cases, the method further comprises enriching a plurality of nucleic acid sequences of interest using the steps of the method. In some cases, the method further comprises sequencing a plurality of enriched nucleic acid sequences of interest. In some cases, the sequencing comprises use of a reversible-dye terminator.

In some aspects, a method is provided for enriching for a nucleic acid sequence of interest in a sample comprising nucleic acids, the method comprising: a) annealing a first oligonucleotide to a nucleic acid fragment comprising the nucleic acid sequence of interest, wherein the first oligonucleotide comprises a 3′ portion complementary to a sequence in the nucleic acid fragment and a 5′ portion comprising a first adaptor sequence; b) extending the first oligonucleotide annealed to the nucleic acid fragment comprising the nucleic acid sequence of interest with a polymerase, thereby generating a first oligonucleotide extension product comprising sequence complementary to the nucleic acid sequence of interest and the first adaptor sequence at a first end; c) annealing a second oligonucleotide to the complement of the nucleic acid sequence of interest in the first oligonucleotide extension product, wherein the second oligonucleotide comprises a 3′ portion complementary to the sequence complementary to the nucleic acid sequence of interest and a 5′ portion comprising a second adaptor sequence; d) extending the second oligonucleotide annealed to the complement of the nucleic acid sequence of interest with a polymerase, thereby generating a second oligonucleotide extension product comprising a sequence complementary to the first adaptor sequence at a first end, the nucleic acid sequence of interest, and the second adaptor sequence at a second end; and e) amplifying the nucleic acid sequence of interest using a first primer that anneals to the complement of the first adaptor sequence and a second primer that anneals to the complement of the second adaptor sequence to enrich for the nucleic acid sequence of interest, thereby generating an enriched nucleic acid sequence of interest. In some cases, the method further comprises, before step a), fragmenting a nucleic acid in the sample, thereby generating the nucleic acid fragment of step a) comprising the nucleic acid sequence of interest. In some cases, the nucleic acid fragment is double-stranded prior to step a). In some cases, the nucleic acid fragment comprises deoxyribonucleic acid (DNA) or ribonucleic acid (RNA). In some cases, the DNA is genomic DNA. In some cases, the DNA is cDNA. In some cases, the 3′ portion of the first oligonucleotide comprises DNA and the 5′ portion of the first oligonucleotide comprises RNA. In some cases, the method further comprises denaturing the double-stranded nucleic acid fragment prior to step a), wherein the nucleic acid fragment comprising the nucleic acid sequence of interest in step a) is single-stranded prior to annealing to the first oligonucleotide. In some cases, the 3′ portion of the first oligonucleotide comprises a random sequence. In some cases, the 3′ portion of the second oligonucleotide comprises a sequence designed to anneal to the nucleic acid sequence of interest. In some cases, the first adaptor sequence and the second adaptor sequence are distinct. In some cases, the first adaptor sequence and/or the second adaptor sequence comprise barcode sequence. In some cases, the first adaptor sequence and/or the second adaptor sequence comprise sequence that can anneal to a sequencing primer, or complement of a sequence that can anneal to a sequencing primer. In some cases, the polymerase is a DNA polymerase.

In some cases, the method further comprises: annealing a plurality of first oligonucleotides to different nucleic acid fragments comprising different nucleic acid sequences of interest, wherein each of the plurality of first oligonucleotides comprises a 3′ portion complementary to a sequence in the nucleic acid fragment among the nucleic acid fragments and a 5′ portion comprising a first adaptor sequence; extending the plurality of first oligonucleotides annealed to the different nucleic acid fragments comprising different nucleic acid sequences of interest with a polymerase, thereby generating a plurality of first oligonucleotide extension products each comprising sequence complementary to the nucleic acid sequences of interest and the first adaptor sequence at a first end; annealing a plurality of second oligonucleotides to the complement of the nucleic acid sequences of interest in the plurality of first oligonucleotide extension products, wherein each of the plurality of second oligonucleotides comprises a 3′ portion complementary to the sequence complementary to the nucleic acid sequence of interest among the nucleic acid sequences of interest and a 5′ portion comprising a second adaptor sequence; extending the plurality of second oligonucleotides annealed to the complement of the nucleic acid sequences of interest in the first oligonucleotide extension products, thereby generating a plurality of second oligonucleotide extension products each comprising a sequence complementary to the first adaptor sequence at a first end, the nucleic acid sequence of interest among the nucleic acid sequences of interest, and the second adaptor sequence at a second end; and amplifying the nucleic acid sequences of interest using a first primer that anneals to the complement of the first adaptor sequence and a second primer that anneals to the complement of the second adaptor sequence to enrich for the nucleic acid sequences of interest, thereby generating enriched nucleic acid sequences of interest. In some cases, the method further comprises prior to step e), digesting the second oligonucleotide extension product with RNase H. In some cases, amplifying in step e) comprises a polymerase chain reaction. In some cases, the method further comprises enriching a plurality of nucleic acid sequences of interest using steps a)-e). In some cases, the method further comprises sequencing the plurality of enriched nucleic acid sequences of interest. In some cases, the nucleic acid sequence of interest comprises a gene. In some cases, the gene is a cancer gene. In some cases, the method further comprises sequencing the enriched nucleic acid sequence of interest. In some cases, the sequencing comprises use of a reversible-dye terminator.

In another aspect, a method is provided for enriching for a nucleic acid sequence of interest in a sample comprising nucleic acids, the method comprising: a) fragmenting the nucleic acids thereby generating nucleic acid fragments, wherein the nucleic acid fragments comprise the nucleic acid sequence of interest; b) ligating a first adaptor sequence to a 5′ end of each of the nucleic acid fragments thereby generating nucleic acid fragments comprising the first adaptor sequence at the 5′ end; c) annealing one or more oligonucleotides to the nucleic acid fragments comprising the first adaptor sequence at the 5′ end, wherein each of the one or more oligonucleotides comprise a 3′ portion that is complementary to a nucleic acid sequence of interest present in one or more of the nucleic acid fragments, and a 5′ portion comprising a second adapter sequence; d) extending the one or more oligonucleotides with a polymerase thereby generating one or more oligonucleotide extension products comprising a sequence complementary to the first adaptor sequence at a first end, a sequence complementary to the nucleic acid sequence of interest and the second adaptor sequence at a second end; and e) amplifying the one or more oligonucleotide extension products using a first primer that anneals to the complement of the first adaptor sequence and a second primer that anneals to the complement of the second adaptor sequence to enrich for the nucleic acid sequence of interest, wherein the nucleic acid sequence of interest comprises a gene fusion or a gene rearrangement. In some cases, the nucleic acids in the sample comprise RNA. In some cases, the RNA is converted to double-stranded cDNA prior to step b). In some cases, the gene fusion or gene rearrangement is in a cancer gene. In some cases, the gene rearrangement is a translocation or an inversion. In some cases, the one or more oligonucleotides anneals to a portion of at least one gene in the gene fusion. In some cases, the at least one gene in the gene fusion is BCR, ABL, ALK, RET, or ROS1. In some cases, the method further comprises sequencing the nucleic acid sequence of interest after step e). In some cases, the method further comprises denaturing the nucleic acid fragments prior to step c), thereby generating single-stranded nucleic acid fragments comprising the first adaptor sequence at the 5′ end. In some cases, the first adaptor sequence, the second adaptor sequence or a combination thereof comprises barcode sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following description that sets forth illustrative embodiments, in which the principles of the invention are utilized, and the accompanying drawings of which:

FIG. 1 depicts selective target enrichment using ligation of a single forward adaptor at the ends of the DNA fragments in the DNA library. The sequence-specific oligonucleotide that anneals to the target region of interest contains a common reverse adaptor sequence at its 5′ end, and following sequence-specific oligonucleotide extension, PCR is performed using a set of primers specific to the forward and reverse adaptors.

FIG. 2 depicts an alternative ligation protocol wherein the DNA-fragment-adaptor complex is denatured following ligation without nick repair and adaptor fill-in, generating ligation products where non-complementary ends exist on each insert.

FIG. 3 depicts selective target enrichment using ligation of partial duplex adaptors. Cleavage of the 5′ end of the long strand of the partial duplex adaptor (and the corresponding complementary sequence of the extended sequence-specific oligonucleotide) by a nucleic acid modifying enzyme specific for double-stranded DNA allows for ligation of a new adaptor pair, and consequently, amplification with primers corresponding to the new adaptors.

FIG. 4 depicts high efficiency NGS library generation using random priming. The oligonucleotide that anneals to the DNA fragment contains a common reverse adaptor sequence at its 5′ end, and following primer extension, PCR is performed using a set of primers specific to the forward and reverse adaptors.

FIG. 5 depicts the Ovation Target Enrichment System workflow.

FIG. 6 depicts Agencourt RNAClean XP Bead purification process overview.

FIG. 7 depicts fragment distribution when 1 μL of a target enriched library is loaded into a Bioanalyzer High Sensitivity DNA Chip.

FIG. 8 depicts stylized qPCR amplification plot.

FIG. 9 depicts enrichment of cDNA from 100 ng universal human reference (UHR) RNA using the Ovation Target Enrichment System.

FIG. 10 depicts selective target enrichment using random priming of a forward adaptor and target-specific priming of a reverse adaptor.

FIG. 11 depicts selective target enrichment using target-specific priming to append a reverse adaptor to nucleic acid fragments followed by ligation to append a forward adaptor.

FIG. 12 depicts selective target enrichment using a transposase-mediated fragmenting and tagging reaction to append a forward adaptor to the ends of nucleic acid fragments followed by target-specific priming to append a reverse adaptor.

FIG. 13 depicts selective target enrichment using a transposase-mediated fragmenting and tagging reaction to append a first adaptor to the end of nucleic acid fragments followed by target-specific priming to append a second adaptor.

FIG. 14 depicts selective target enrichment using random priming to append a first adaptor nucleic acid fragments followed by target-specific priming to append a second adaptor.

### DETAILED DESCRIPTION OF THE INVENTION

### General

The methods provided herein can be used for the selective enrichment of a plurality of defined target sequences from complex DNA with a set of common primers and adaptors, thus circumventing the need for multiplex PCR and multiple primer pairs. A multiplicity of target regions of interest are envisioned: for example, the regions of interest can represent all known coding regions, the entire exome, selected regions of coding genomic regions representing selected pathways, selected genomic regions known to comprise genomic variation related to altered phenotype, entire or selected regions of a specific chromosome, and the like. The methods described herein can be used for high efficiency nucleic acid library production as well. A target region of interest can include sequence used for primer annealing (e.g., a probe “landing zone”).

Altogether, the methods described herein can create a simple, low cost, high throughput system for target enrichment and library preparation.

Reference will now be made in detail to exemplary embodiments described herein. While the disclosed methods and compositions will be described in conjunction with the exemplary embodiments, it will be understood that these exemplary embodiments are not intended to limit the invention. Methods and compositions described herein encompass alternatives, modifications and equivalents.

Provided herein are methods and compositions for the enrichment of specific target sequences of interest from a sample comprising nucleic acids. The methods described herein can enrich target sequences using conventional duplex adaptors and/or partial duplex adaptors, sequence specific oligonucleotides, restriction enzymes and ligation. The methods can further enable enrichment of target sequences from specific strands of template nucleic acids which can be further amplified using a variety of amplification methods. Provided herein are methods for high efficiency generation of libraries comprising specific nucleic acid sequences of interest.

Provided herein are methods and compositions for the enrichment of target nucleic acid sequences from a sample comprising nucleic acids. The method can comprise fragmenting nucleic acids in an input sample to generate nucleic acid fragments. The nucleic acids can be DNA, or RNA. The nucleic acids can be single or double stranded. The DNA can be genomic DNA or cDNA or any combination thereof. The nucleic acids in an input sample can be double stranded DNA. Fragmentation of the nucleic acids can be achieved through methods known in the art. Fragmentation can be through physical fragmentation methods and/or enzymatic fragmentation methods. Physical fragmentation methods can include nebulization, sonication, and/or hydrodynamic shearing. The fragmentation can be accomplished mechanically comprising subjecting the nucleic acids in the input sample to acoustic sonication. The fragmentation can comprise treating the nucleic acids in the input sample with one or more enzymes under conditions suitable for the one or more enzymes to generate double-stranded nucleic acid breaks. Examples of enzymes useful in the generation of nucleic acid or polynucleotide fragments include sequence specific and non-sequence specific nucleases. Non-limiting examples of nucleases include DNase I, Fragmentase, restriction endonucleases, variants thereof, and combinations thereof. Reagents for carrying out enzymatic fragmentation reactions can be commercially available (e.g, from New England Biolabs). For example, digestion with DNase I can induce random double-stranded breaks in DNA in the absence of Mg⁺⁺ and in the presence of Mn⁺⁺. Fragmentation can comprise treating the nucleic acids in the input sample with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence. The method can include the step of size selecting the fragments via standard methods known in the art such as column purification or isolation from an agarose gel.

The nucleic acids in the input sample can be fragmented into a population of fragmented nucleic acid molecules or polynucleotides of one or more specific size range(s). The fragments can have an average length from about 10 to about 10,000 nucleotides. The fragments can have an average length from about 50 to about 2,000 nucleotides. The fragments can have an average length from about 100 to about 2,500, about 10 to about 1,000, about 10 to about 800, about 10 to about 500, about 50 to about 500, about 50 to about 250, or about 50 to about 150 nucleotides. The fragments can have an average length less than 10,000 nucleotides, such as less than 5,000 nucleotides, less than 2,500 nucleotides, less than 2,500 nucleotides, less than 1,000 nucleotides, less than 500 nucleotides, such as less than 400 nucleotides, less than 300 nucleotides, less than 200 nucleotides, or less than 150 nucleotides.

Fragmentation of the nucleic acids can be followed by end repair of the nucleic acid fragments. End repair can include the generation of blunt ends, non-blunt ends (i.e sticky or cohesive ends), or single base overhangs such as the addition of a single dA nucleotide to the 3′-end of the nucleic acid fragments, by a polymerase lacking 3′-exonuclease activity. End repair can be performed using any number of enzymes and/or methods known in the art including, but not limited to, commercially available kits such as the Encore™ Ultra Low Input NGS Library System I. End repair can be performed on double stranded DNA fragments to produce blunt ends wherein the double stranded DNA fragments contain 5′ phosphates and 3′ hydroxyls. The double-stranded DNA fragments can be blunt-end polished (or “end repaired”) to produce DNA fragments having blunt ends, prior to being joined to adapters. Generation of the blunt ends on the double stranded fragments can be generated by the use of a single strand specific DNA exonuclease such as for example exonuclease 1, exonuclease 7 or a combination thereof to degrade overhanging single stranded ends of the double stranded products. The double stranded DNA fragments can be blunt ended by the use of a single stranded specific DNA endonuclease, for example, but not limited to, mung bean endonuclease or 51 endonuclease. The double stranded products can be blunt ended by the use of a polymerase that comprises single stranded exonuclease activity such as for example T4 DNA polymerase, or any other polymerase comprising single stranded exonuclease activity or a combination thereof to degrade the overhanging single stranded ends of the double stranded products. The polymerase comprising single stranded exonuclease activity can be incubated in a reaction mixture that does or does not comprise one or more dNTPs. A combination of single stranded nucleic acid specific exonucleases and one or more polymerases can be used to blunt end the double stranded fragments generated by fragmenting the sample comprising nucleic acids. The nucleic acid fragments can be made blunt ended by filling in the overhanging single stranded ends of the double stranded fragments. For example, the fragments can be incubated with a polymerase such as T4 DNA polymerase or Klenow polymerase or a combination thereof in the presence of one or more dNTPs to fill in the single stranded portions of the double stranded fragments. The double stranded DNA fragments can be made blunt by a combination of a single stranded overhang degradation reaction using exonucleases and/or polymerases, and a fill-in reaction using one or more polymerases in the presence of one or more dNTPs.

In some embodiments, the 5′ and/or 3′ end nucleotide sequences of fragmented nucleic acids are not modified or end-repaired prior to appending with the adapter oligonucleotides of the present invention. For example, fragmentation by a restriction endonuclease can be used to leave a predictable overhang, followed by ligation with one or more adapter oligonucleotides comprising an overhang complementary to the predictable overhang on a nucleic acid fragment. In another example, cleavage by an enzyme that leaves a predictable blunt end can be followed by ligation of blunt-ended nucleic acid fragments to adapter oligonucleotides comprising a blunt end. In some embodiments, end repair can be followed by an addition of 1, 2, 3, 4, 5, 6, 7, 8, 9, 10, 11, 12, 13, 14, 15, 16, 17, 18, 19, 20 or more nucleotides, such as one or more adenine, one or more thymine, one or more guanine, or one or more cytosine, to produce an overhang. Nucleic acid fragments having an overhang can be joined to one or more adapter oligonucleotides having a complementary overhang, such as in a ligation reaction. For example, a single adenine can be added to the 3′ ends of end repaired DNA fragments using a template independent polymerase, followed by ligation to one or more adapters each having a thymine at a 3′ end. In some embodiments, adapter oligonucleotides can be joined to blunt end double-stranded nucleic acid fragments which have been modified by extension of the 3′ end with one or more nucleotides followed by 5′ phosphorylation. In some cases, extension of the 3′ end can be performed with a polymerase such as for example Klenow polymerase or any of the suitable polymerases provided herein, or by use of a terminal deoxynucleotide transferase, in the presence of one or more dNTPs in a suitable buffer containing magnesium. In some embodiments, nucleic acid fragments having blunt ends can be joined to one or more adapters comprising a blunt end. Phosphorylation of 5′ ends of nucleic acid fragments can be performed for example with T4 polynucleotide kinase in a suitable buffer containing ATP and magnesium. The fragmented nucleic acid molecules may optionally be treated to dephosphorylate 5′ ends or 3′ ends, for example, by using enzymes known in the art, such as phosphatases.

The methods described herein for enriching for target nucleic acid sequences further comprise appending a first adaptor to the nucleic acid fragments generated by the methods described herein. Appending the first adaptor to the nucleic acid fragments generated by methods described herein can be achieved using a ligation reaction or a priming reaction. In one embodiment, appendage of a first adaptor to the nucleic acid fragments comprises ligation. In one embodiment, ligation of the first adaptor to the nucleic acid fragments can be following end repair of the nucleic acid fragments. In another embodiment, the ligation of the first adaptor to the nucleic acid fragments can be following generation of the nucleic acid fragments without end repair of the nucleic acid fragments. The first adaptor can be any type of adaptor known in the art including, but not limited to, conventional duplex or double stranded adaptors in which the adaptor comprises two complementary strands. In one embodiment, the first adaptor can be a forward adaptor. In a preferred embodiment, the first adaptor can be a double stranded DNA adaptor. In one embodiment, the first adaptor can be an oligonucleotide of known sequence and, thus, allow generation and/or use of sequence specific primers for amplification and/or sequencing of any polynucleotides to which the first adaptor(s) is appended or attached. In one embodiment, the first adaptor can be a conventional duplex adaptor, wherein the first adaptor comprises sequence well known in the art. In a preferred embodiment, the first adaptor can be appended to the nucleic acid fragments generated by the methods described herein in multiple orientations. In a preferred embodiment, the methods described herein can involve the use of a first duplex adaptor comprising double stranded DNA of known sequence that is blunt ended and can bind to the double stranded nucleic acid fragments generated by the methods described herein in one of two orientations. In one embodiment, the first adaptor can be ligated to each of the nucleic acid fragments such that each of the nucleic acid fragments comprises the same first adaptor. In other words, each of the nucleic acid fragments comprises a common first adaptor. In another embodiment, a first adaptor can be appended or ligated to a library of nucleic acid fragments generated by the methods described herein such that each nucleic acid fragment in the library of nucleic acid fragments comprises the first adaptor ligated to one or both ends.

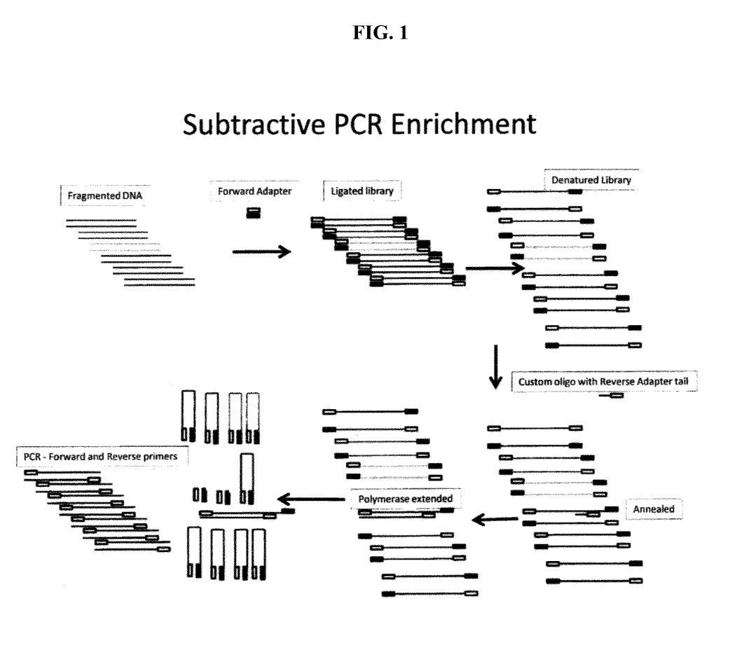

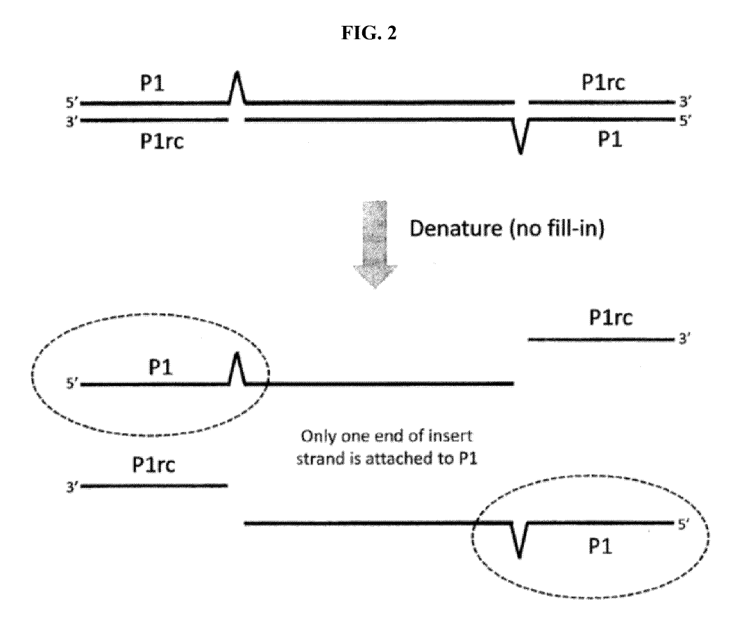

In one embodiment, the first adaptor can be ligated or appended to the 5′ and/or 3′ ends of the nucleic acid fragments generated by the methods described herein. The first adaptor can comprise two strands wherein each strand comprises a free 3′ hydroxyl group but neither strand comprises a free 5′ phosphate. In one embodiment, the free 3′ hydroxyl group on each strand of the first adaptor can be ligated to a free 5′ phosphate present on either end of the nucleic acid fragments of the present invention. In this embodiment, the first adaptor comprises a ligation strand and a non-ligation strand whereby the ligation strand can be ligated to the 5′phosphate on either end of the nucleic acid fragment while a nick or gap can be present between the non-ligation strand of the first adaptor and the 3′ hydroxyl on either end of the nucleic acid fragment. In one embodiment, the nick or gap can be filled in by performing a gap repair reaction. In one embodiment, the gap repair can be performed with a DNA dependent DNA polymerase with strand displacement activity. In one embodiment, the gap repair can be performed using a DNA dependent DNA polymerase with weak or no strand displacement activity. In one embodiment, the ligation strand of the first adaptor can serve as the template for the gap repair or fill-in reaction. In this embodiment, the gap repair or fill-in reaction comprises an extension reaction wherein the ligation strand of the first adaptor serves as a template and leads to the generation of nucleic acid fragments with complementary termini or ends as depicted, for example, in FIG. 1. In one embodiment, the gap repair can be performed using Taq DNA polymerase. In one embodiment, the ligation of the first adaptor to the nucleic acid fragments generated by the methods described herein may not be followed by gap repair as depicted, for example, in FIG. 2. In this embodiment, the nucleic acid fragments comprise first adaptor sequence ligated only at the 5′ end of each strand.

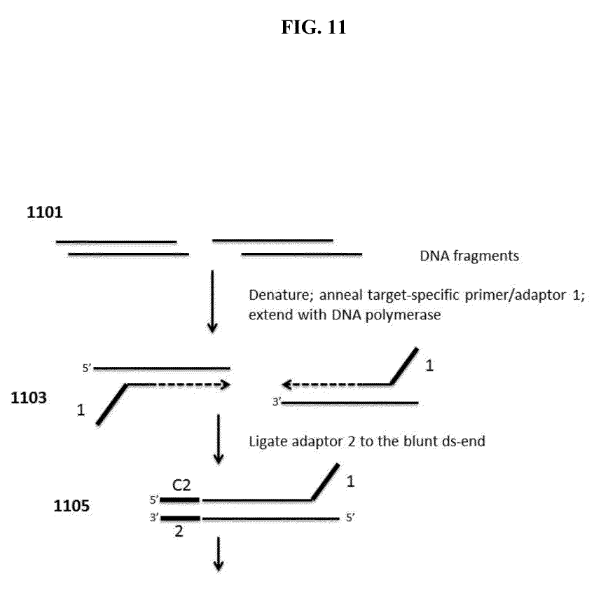

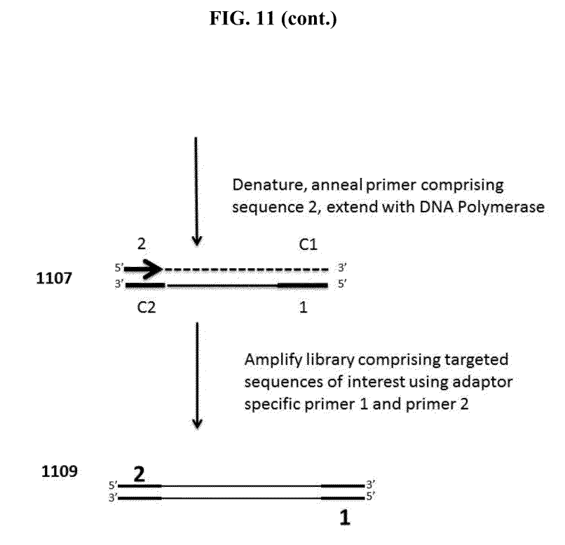

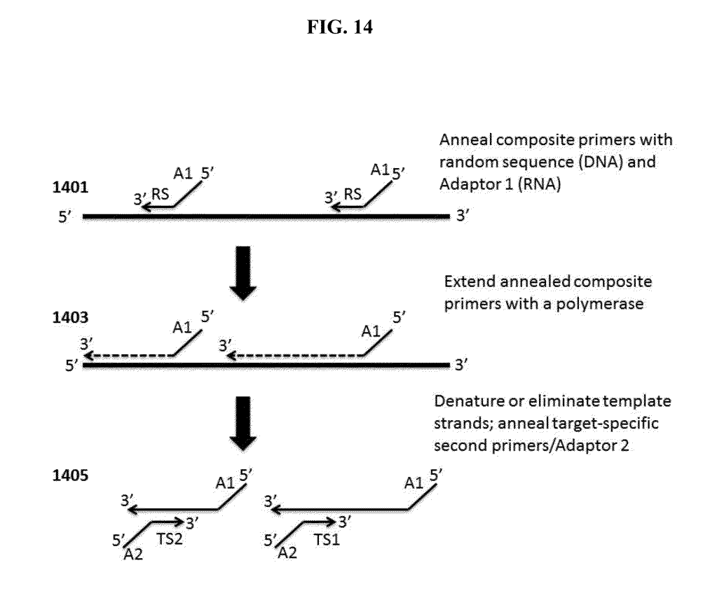

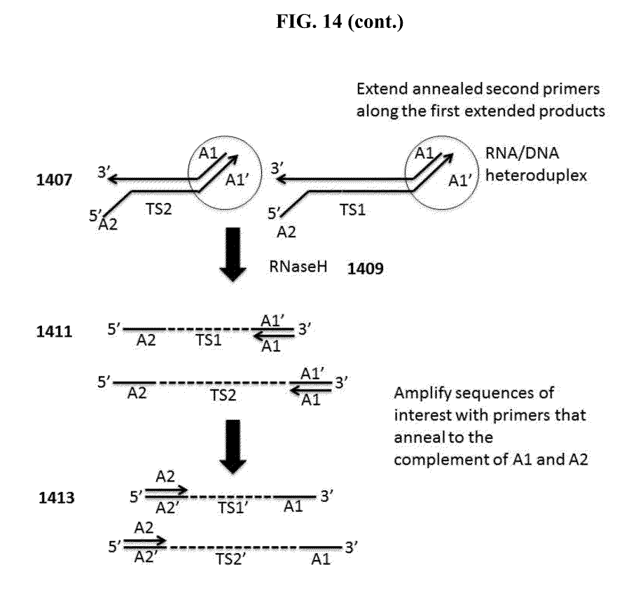

Appending a first adaptor to a nucleic acid fragment can comprise a priming reaction, for example, in FIG. 11. An oligonucleotide comprising, at a first end, sequence complementary to a target sequence of interest present in the nucleic acid fragment and, at a second end, sequence comprising the first adaptor sequence, wherein the first adaptor sequence is not complementary to the target nucleic acid can be annealed to the nucleic acid fragment. In some examples, the first adaptor can be appended by annealing a random primer containing random sequence to the nucleic acid fragment, as shown in FIG. 14. In this example, the random primer can be a composite primer (e.g., a 3′ portion containing DNA and a 5′ portion containing RNA). The nucleic acid fragment can be single stranded. A double-stranded nucleic acid fragment can be denatured to generate a single-stranded nucleic acid fragment. Denaturation can be achieved using any of the methods known in the art including, but not limited to, physical, thermal, and/or chemical denaturation. Denaturation can be achieved using thermal or heat denaturation. The single stranded target nucleic acid sequence of interest can be present in one or more single stranded nucleic acid fragments. Different or distinct target nucleic acid sequences of interest can be present in one or more of the single stranded nucleic acid fragments. One or more oligonucleotides can comprise sequence complementary to the same sequence of interest present in one or more single stranded nucleic acid fragments. The one or more oligonucleotides can comprise sequence that is complementary to different parts or regions of the same sequence of interest. The different regions can be adjacent to each other. The different regions can be non-adjacent to each other. The one or more oligonucleotides that comprise sequence complementary to the same target nucleic acid sequence of interest can further comprise the same first adaptor sequence. One or more oligonucleotides can comprise sequence complementary to different or distinct sequences of interest which can be present in one or more single stranded nucleic acid fragments. The one or more oligonucleotides that comprise sequence complementary to different or distinct target nucleic acid sequences of interest can further comprise the same first adaptor sequence. The sequence complementary to the target sequence of interest can be at the 3′ end of the oligonucleotide and the first adaptor sequence can be at the 5′end of the oligonucleotide. The first adaptor sequence can be non-complementary to the target nucleic acid sequence of interest. The first adaptor sequence can serve as a tail. The first adaptor sequence can be a conventional adaptor sequence. The first adaptor can comprise reverse adaptor sequence. The first adaptor sequence can be of known sequence and, thus, can allow generation and/or use of sequence specific primers for amplification and/or sequencing of any polynucleotides to which the first adaptor sequence is appended or attached. The oligonucleotide can be annealed to the nucleic acid fragments without prior denaturation. Annealing of the oligonucleotide can be via formation of a triple helix or triplex between the oligonucleotide and a double stranded nucleic acid fragment. The double stranded nucleic acid fragment can comprise a sequence of interest and can be present amongst a plurality of double stranded nucleic acid fragments. The oligonucleotide can comprise sequence complementary to the sequence of interest in the double stranded nucleic acid fragment. The use of the oligonucleotide comprising sequence complementary to a target sequence of interest present in a nucleic acid fragment amongst one or more of a plurality of nucleic acid fragments can allow for selective binding and subsequent enrichment of the nucleic acid fragment using the methods described herein. In some cases, the annealing can occur in solution.

Following annealing of the oligonucleotide as described above, a polymerase can be used to extend the oligonucleotide. The polymerase can be a DNA dependent DNA polymerase. The DNA dependent DNA polymerase can be any of the DNA dependent DNA polymerases as described herein and extension of the oligonucleotide can be by any of the methods known in the art. An oligonucleotide comprising the first adaptor sequence, wherein the first adaptor sequence is not complementary to the target nucleic acid, and sequence complementary to a target sequence of interest present in a nucleic acid fragment can be annealed to the nucleic acid fragment and extended with a polymerase to generate an oligonucleotide extension product comprising the first adaptor sequence at a first end. The nucleic acid fragment can be present amongst a plurality of nucleic acid fragments. In this embodiment, the oligonucleotide extension product can only be generated for a nucleic acid fragment that contains the target sequence of interest.

After generating a nucleic acid molecule comprising a first adaptor sequence at a first end by any of the methods described above (e.g., ligation or priming reaction), a second adaptor sequence may be appended to the nucleic acid molecule at a second end. The second adaptor sequence can be appended to the nucleic acid molecule at a second end by ligation, for example, as shown in FIG. 11. The second adaptor sequence can be appended to the nucleic acid molecule at a second end by a priming reaction.

In some embodiments, the second adaptor sequence is appended to a nucleic acid fragment comprising a first adaptor at a first end by a priming reaction. In one embodiment, the nucleic acid fragment is double stranded. In one embodiment, the double stranded nucleic acid fragment is denatured prior to the priming reaction to generate a single stranded nucleic acid fragment comprising a first adaptor sequence at a first end. Denaturation can be achieved using any of the methods as described above. In one embodiment, denaturation of the first adaptor-nucleic acid fragment complex generates single stranded nucleic acid fragments comprising first adaptor sequence at only the 5′ end of the nucleic acid fragments as depicted, for example, in FIG. 2. In another embodiment, denaturation of the first adaptor-nucleic acid fragment complex generates single stranded nucleic acid fragments comprising first adaptor sequence at both the 5′end and 3′ end of the nucleic acid fragments as depicted, for example, in FIG. 1.

In one embodiment, the nucleic acid fragments comprising first adaptor sequence appended to either the 5′ end or both the 5′ and 3′ end can be denatured to generate single stranded nucleic acid fragments comprising first adaptor sequence appended to either the 5′ end or both the 5′ and 3′ end. In one embodiment, the methods of the present invention described herein can be used to generate a plurality of single stranded nucleic acid fragments comprising first adaptor sequence appended to either the 5′ end or both the 5′ and 3′ end.

In some embodiments, a second adaptor sequence may be appended to a single stranded nucleic acid fragment comprising first adaptor sequence appended to either the 5′ end or both the 5′ and 3′ ends. In one embodiment, appending a second adaptor sequence to a single stranded nucleic acid fragment comprising first adaptor sequence appended to either the 5′ end or both the 5′ and 3′ ends can comprise a priming reaction. In this embodiment, an oligonucleotide comprising, at a first end, sequence complementary to a target sequence of interest present in a single stranded nucleic acid fragment, and at a second end, sequence from a second adaptor, wherein the second adaptor sequence is not complementary to the target nucleic acid can be annealed to the single stranded nucleic acid fragments. In one embodiment, the second adaptor sequence can be sequence from a reverse adaptor. In one embodiment, the target nucleic acid sequence of interest can be present in one or more of the single stranded nucleic acid fragments. In one embodiment, different or distinct target nucleic acid sequences of interest can be present in one or more of the single stranded nucleic acid fragments. In one embodiment, one or more oligonucleotides can comprise sequence complementary to the same sequence of interest present in one or more single stranded nucleic acid fragments. In this embodiment, the one or more oligonucleotides can comprise sequence that is complementary to different parts or regions of the same sequence of interest. In one embodiment, the different regions can be adjacent to each other. In one embodiment, the different regions can be non-adjacent to each other. In a preferred embodiment, the one or more oligonucleotides that comprise sequence complementary to the same target nucleic acid sequence of interest further comprise the same second adaptor sequence. In another embodiment, one or more oligonucleotides can comprise sequence complementary to different or distinct sequences of interest which can be present in one or more single stranded nucleic acid fragments. In a preferred embodiment, the one or more oligonucleotides that comprise sequence complementary to different or distinct target nucleic acid sequences of interest further comprise the same second adaptor sequence. In one embodiment, the sequence complementary to the target sequence of interest can be at the 3′end of the oligonucleotide and the second adaptor sequence can be at the 5′ end of the oligonucleotide. In a preferred embodiment, the second adaptor sequence is non-complementary to the target nucleic acid sequence of interest. In this manner, the second adaptor sequence serves as a tail. The second adaptor sequence can be a conventional adaptor sequence. In a preferred embodiment, the second adaptor sequence can be a conventional adaptor sequence that is different than or distinct from the sequence of the first adaptor appended to the single stranded nucleic acid fragment as described above. In one embodiment, the second adaptor sequence can be of known sequence and, thus, allow generation and/or use of sequence specific primers for amplification and/or sequencing of any polynucleotides to which the second adaptor sequence is appended or attached. In a separate embodiment, the oligonucleotide can be annealed to the nucleic acid fragments comprising the first adaptor sequence appended to either the 5′ end or both the 5′ and 3′ end without prior denaturation. In this embodiment, annealing of the oligonucleotide can be via formation of a triple helix or triplex between the oligonucleotide and a double stranded nucleic acid fragment comprising the first adaptor sequence appended to either the 5′ end or both the 5′ and 3′ ends of the double stranded nucleic acid fragment. In this embodiment, the double stranded nucleic acid fragment comprises a sequence of interest and can be present amongst a plurality of double stranded nucleic acid fragments comprising first adaptor sequence appended to either the 5′ end or both the 5′ and 3′ end. Further to this embodiment, the oligonucleotide comprises sequence complementary to the sequence of interest in the double stranded nucleic acid fragment. Overall, the use of the oligonucleotide comprising sequence complementary to a target sequence of interest present in a nucleic acid fragment amongst one or more or a plurality of nucleic acid fragments allows for selective binding and subsequent enrichment of said nucleic acid fragment using the methods described herein.

Following annealing of the oligonucleotide as described above, a polymerase can be used to extend the oligonucleotide. In one embodiment, the polymerase can be a DNA dependent DNA polymerase. In one embodiment, the DNA dependent DNA polymerase can be any of the DNA dependent DNA polymerases as described herein and extension of the oligonucleotide can be by any of the methods known in the art. In one embodiment, an oligonucleotide comprising the second adaptor sequence, wherein the second adaptor sequence is not complementary to the target nucleic acid, and sequence complementary to a target sequence of interest present in a nucleic acid fragment comprising a first adaptor appended to one and/or both ends can be annealed to the nucleic acid fragment and extended with a polymerase to generate an oligonucleotide extension product comprising the first adaptor sequence at a first end and the second adaptor sequence at a second end. In some examples, the oligonucleotide extension product may form an RNA/DNA heteroduplex, for example, as depicted in FIG. 14. In one embodiment, the nucleic acid fragment can be present amongst a plurality of nucleic acid fragments comprising first adaptor appended to one and/or both ends. In this embodiment, the oligonucleotide extension product can only be generated for a nucleic acid fragment that contains the target sequence of interest.

A second adaptor sequence can be appended to a second end of a nucleic acid molecule comprising a first adaptor sequence at a first end by ligation as shown in, for example, FIG. 11. The nucleic acid molecule can be an oligonucleotide extension product comprising a first adaptor sequence at a first end as disclosed herein. The second adaptor sequence can be sequence from a forward adaptor. Ligation of the second adaptor to the nucleic acid fragment can follow end repair of the oligonucleotide extension product. Ligation of the second adaptor to the oligonucleotide extension product can follow generation of the oligonucleotide extension product without end repair of the oligonucleotide extension product. The second adaptor can be any type of adaptor known in the art including, but not limited to, conventional duplex or double stranded adaptors in which the adaptor comprises two complementary strands. The second adaptor can be a double stranded DNA adaptor. The second adaptor can be a single stranded DNA adaptor. The second adaptor can be an oligonucleotide of known sequence and, thus, allow generation and/or use of sequence specific primers for amplification and/or sequencing of any polynucleotides to which the second adaptor(s) is appended or attached. The second adaptor can be a conventional duplex adaptor, wherein the second adaptor comprises sequence well known in the art. The second adaptor can be appended to the oligonucleotide extension product generated by the methods described herein in multiple orientations. The methods described herein can involve the use of a second duplex adaptor comprising double stranded DNA of known sequence that is blunt ended and can bind to the oligonucleotide extension product comprising a first adaptor at a first end generated by the methods described herein in one of two orientations. The second adaptor can be ligated to each of the oligonucleotide extension products such that each of the oligonucleotide extension products comprises the same second adaptor. Each of the oligonucleotide extension products can comprise a common second adaptor. A second adaptor can be appended or ligated to a library of oligonucleotide extension products generated by the methods described herein such that each oligonucleotide extension products in the library of oligonucleotide extension products comprises the second adaptor ligated to one or both ends.

The second adaptor can be ligated or appended to a 5′ and/or 3′ end of the oligonucleotide extension product generated by the methods described herein. The second adaptor can comprise two strands wherein each strand comprises a free 3′ hydroxyl group but neither strand comprises a free 5′ phosphate. The free 3′ hydroxyl group on each strand of the second adaptor can be ligated to a free 5′ phosphate present on either end of the oligonucleotide extension products. The second adaptor can comprise a ligation strand and a non-ligation strand whereby the ligation strand can be ligated to the 5′phosphate on either end of the oligonucleotide extension product while a nick or gap can be present between the non-ligation strand of the first adaptor and the 3′ hydroxyl on either end of the oligonucleotide extension product. The nick or gap can be filled in by performing a gap repair reaction. The gap repair can be performed with a DNA dependent DNA polymerase with strand displacement activity. The gap repair can be performed using a DNA dependent DNA polymerase with weak or no strand displacement activity. The ligation strand of the second adaptor can serve as the template for the gap repair or fill-in reaction. The gap repair or fill-in reaction can comprise an extension reaction wherein the ligation strand of the second adaptor serves as a template and leads to the generation of oligonucleotide extension products with complementary termini or ends. The gap repair can be performed using Taq DNA polymerase. In some cases, the ligation of the second adaptor to the nucleic acid fragments generated by the methods described herein may not be followed gap repair. In this embodiment, the oligonucleotide extension products comprise second adaptor sequence ligated only at the 5′ end of each strand.

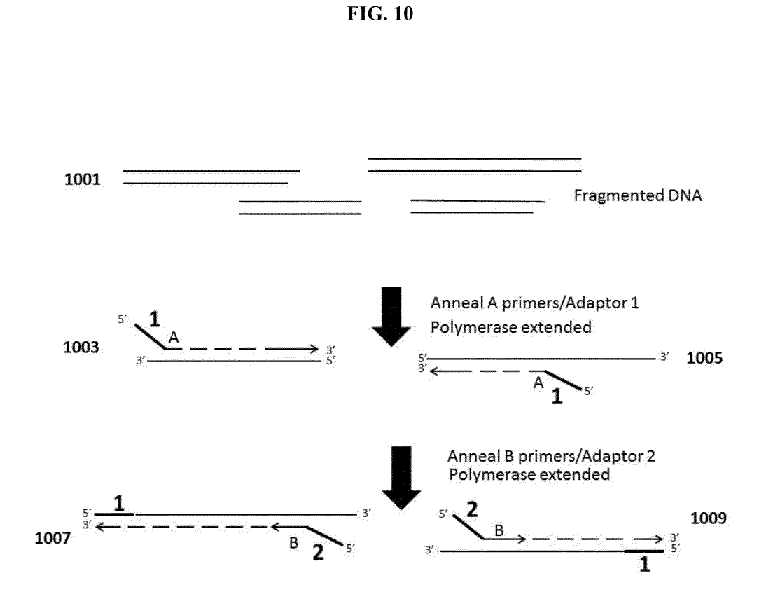

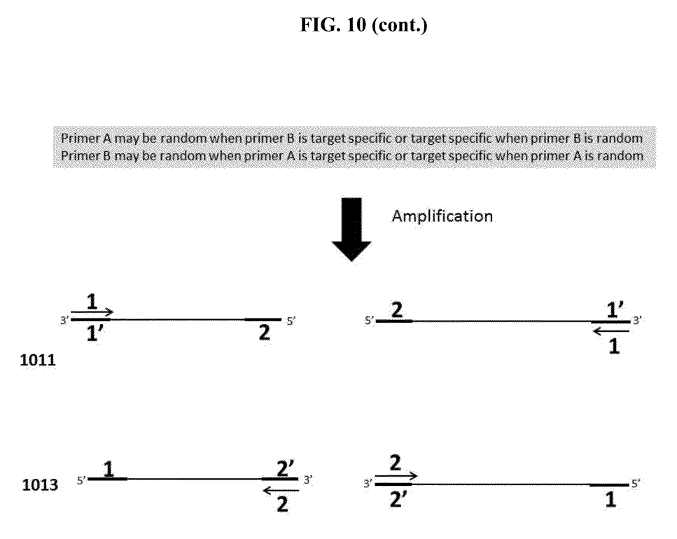

Methods provided herein can be used to generate nucleic acid molecules (i.e., oligonucleotide extension products) comprising a first adaptor sequence at a first end and a second adaptor sequence at a second end. Any method or any combinations of methods as disclosed herein can be used to append one or more adaptors to one or more ends of a nucleic acid fragment. The first adaptor and the second adaptor can be appended by ligation. The first adaptor and the second adaptor can be appended by a priming reaction as shown, for example, in FIG. 10. The first adaptor can be appended by ligation and the second adaptor can be appended by a priming reaction. The first adaptor can be appended by a priming reaction and the second adaptor can be appended by ligation. In one example, the first adaptor can be appended by annealing a primer comprising a target specific sequence at the 3′-end and the first adaptor sequence at a second end, to the nucleic acid of interest followed by extension of the primer with DNA polymerase to generate a partial duplex comprising a blunt end. In this example, the second adaptor can be appended to the partial duplex by ligating the second adaptor to the blunt end without the need for an end repair process, as shown in FIG. 11.

In one embodiment, the oligonucleotide extension products comprising a first adaptor at a first end and a second adaptor at a second end as generated by the methods described herein can be subjected to an amplification reaction. In one embodiment, the amplification reaction can be exponential, and can be carried out at various temperature cycles or can be isothermal. In one embodiment, the amplification can be polymerase chain reaction. In one embodiment, the amplification reaction can be isothermal. In one example, the oligonucleotide extension product can be separated from the template nucleic acid fragment in order to generate a single stranded oligonucleotide extension product with first adaptor sequence on the 5′ end and second adaptor sequence on the 3′ end. The single stranded oligonucleotide extension product can then be amplified using a first primer comprising sequence complementary to the first adaptor and a second primer comprising sequence complementary to the second adaptor sequence. In an alternative example, the oligonucleotide extension product can be separated from the template nucleic acid fragment in order to generate a single stranded oligonucleotide extension product with a first adaptor sequence on the 3′ end and a second adaptor sequence on the 5′ end. The single stranded oligonucleotide extension product can then be amplified using a first primer that anneals to the complement of the second adaptor sequence and a second primer that anneals to the complement of the first adaptor sequence. In this manner only oligonucleotide extension products comprising both the first and the second adaptor sequence will be amplified and thus enriched. In one embodiment, the first adaptor and/or the second adaptor sequence can comprise an identifier sequence. In one embodiment, the identifier sequence can be barcode sequence. In one embodiment, the barcode sequence can be the same or different for the first adaptor and the second adaptor sequence. In one embodiment, the first adaptor and/or the second adaptor sequence can comprise sequence that can be used for downstream applications such as, for example, but not limited to, sequencing. In one embodiment, the first adaptor and/or the second adaptor sequence can comprise flow cell sequences which can be used for sequencing with the sequencing method developed by Illumina and described herein.

In an aspect of the disclosure, a method for enriching a nucleic acid sequence of interest in a sample comprising nucleic acids is provided comprising combining a transposome comprising a transposase and a transposon sequence with nucleic acids in the sample. The nucleic acids can be fragmented generating a nucleic acid fragment comprising the nucleic acid sequence of interest. The transposon sequence can be appended to a first end of the nucleic acid fragment generating a nucleic acid fragment comprising a transposon sequence at a first end. A “transposase” can be any enzyme that catalyzes the transfer of a transposon (or transposable element) from one region of the genome to another. A transposase enzyme (as part of a transposome complex) can be used to tag a nucleic acid molecule with the transposon by fragmenting the nucleic acid to generate nucleic acid fragments and by appending a transposon sequence to the nucleic acid fragments at a first end thereby generating nucleic acid fragments comprising the transposon sequence at a first end. The transposase can form a functional complex with a transposon sequence that is capable of catalyzing a transposition reaction. The transposase can bind to the transposon sequence and append the transposon sequence to the nucleic acid molecules in a process sometimes termed “tagmentation.” Tagmentation can comprise a fragmentation step and an appending step. The fragmentation step and the appending step can be carried out by the transposase enzyme. The fragmenting step and the appending step can be simultaneous or near simultaneous. Fragmentation can generate nucleic acid fragments of various lengths as provided herein. The fragmenting step and appending step can be random. The fragmenting step and the appending step can be site-selective. When fragmentation of a nucleic acid is carried out by the transposase enzyme, it may not be necessary to perform other fragmentation steps as disclosed herein (e.g., mechanical shearing, sonicating, etc).

Any transposase that can tag a nucleic acid molecule with a transposon sequence may be used. Methods of tagging nucleic acid molecules with transposases are known in the art and kits for performing such methods are commercially available (e.g., Nextera DNA-sample prep kit from Epicentre). In some examples, the transposase enzyme is the Tn5 transposase. In other examples, the transposase enzyme may be Tn10 transposase, Mu transposase, or P-element transposase.

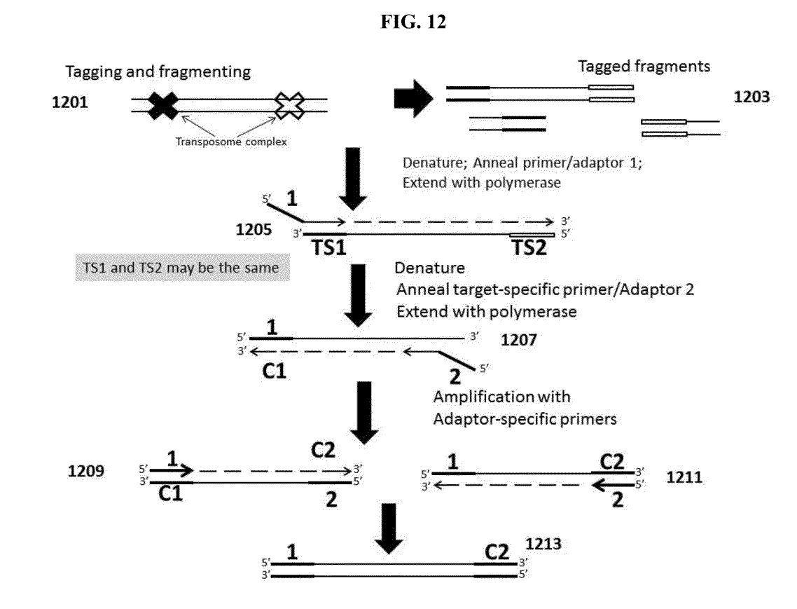

The nucleic acid fragment may be double stranded. The double stranded nucleic acid fragment can be denatured, thereby generating single stranded nucleic acid fragments. The transposon sequence can be appended to a first end of the nucleic acid fragment. The transposon sequence can have a known sequence. Tailed primers can be used to append an adaptor to the nucleic acid fragment. One or more first oligonucleotides can be annealed to the nucleic acid fragments comprising the transposon sequence at a first end. The one or more first oligonucleotides can comprise a 3′ portion that is complementary to the transposon sequence and a 5′ portion comprising a first adaptor sequence. The one or more first oligonucleotides annealed to the transposon sequence can be extended with a polymerase, thereby generating one or more first oligonucleotide extension products comprising sequence complementary to the nucleic acid fragments and the first adaptor sequence at the first end. The polymerase may be a DNA polymerase. Any method of annealing and extending (e.g., a priming reaction) as disclosed herein may be used. After generating a nucleic acid fragment comprising a first adaptor at a first end by the methods described herein, any method of appending a second adaptor to the nucleic acid fragment can be used (e.g., ligation or priming reaction). One or more second oligonucleotides can be annealed to the nucleic acid sequence of interest in the nucleic acid fragments comprising the first adaptor sequence at the first end. The one or more second oligonucleotides can comprise a 3′ portion that is complementary to the nucleic acid sequence of interest and a 5′ portion comprising a second adaptor sequence. Each of the one or more second oligonucleotides can comprise a 3′ portion that is complementary to a portion of a different nucleic acid sequence of interest in the nucleic acid fragments and a 5′ portion comprising the second adaptor sequence. The one or more second oligonucleotides can be extended, thereby generating one or more second oligonucleotide extension products comprising sequence complementary to the first adaptor sequence at the first end, the sequence complementary to the nucleic acid sequence of interest, and the second adaptor sequence at the second end. The one or more second oligonucleotide extension products can be amplified using a first primer that anneals to the complement of the first adaptor sequence and a second primer that anneals to the complement of the second adaptor sequence to enrich for the nucleic acid sequence of interest. A library of nucleic acid sequences may be created. FIG. 12 illustrates an example of selective target enrichment using a transposome-mediated tagmentation protocol.

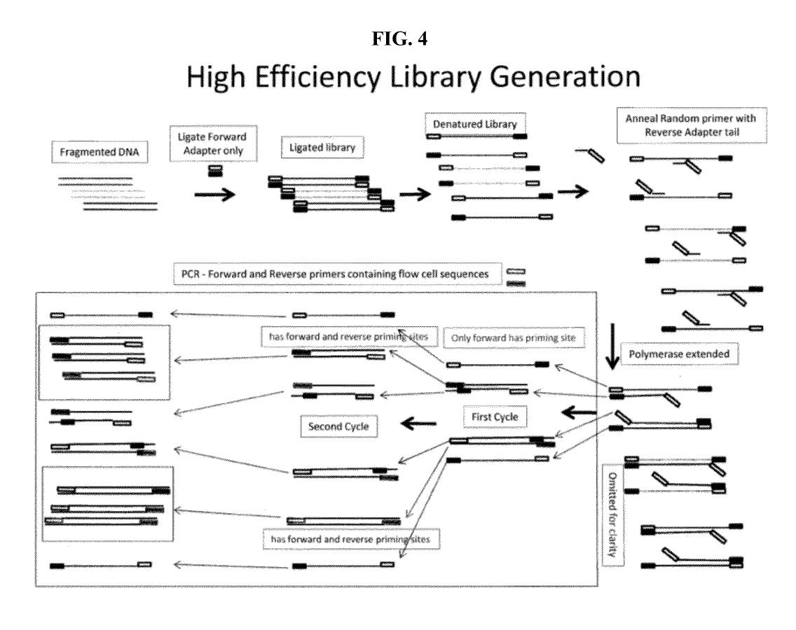

In an alternate embodiment, the methods of the present invention can be used to generate a library of nucleic acid fragments or inserts wherein each nucleic acid fragment comprises an adaptor at one or both ends. In one embodiment, the adaptors can be present at both ends and can be distinct from each other. In one embodiment, the adaptors can be present at both ends and can comprise the same adaptor sequence. The generation of the library comprising nucleic acid inserts with distinct adaptors at both ends can involve the methods for generating oligonucleotide extension products comprising first adaptor sequence on one end and second adaptor sequence on the other end as described herein with the exception that the oligonucleotide that binds to the nucleic acid fragments and can be extended comprises random sequence. The first adaptor may be appended to the nucleic acid fragment by ligation and the second adaptor may subsequently be appended by a priming reaction, as described herein. The oligonucleotide can comprise random sequence at the 3′ portion that is hybridizable to one or more nucleic acid fragments and can further comprises second adaptor sequence at the 5′-portion. Extension of the oligonucleotide along the nucleic acid fragment and the corresponding first adaptor can generates a product, or products, comprising the second adaptor at one end and a sequence complementary to the first adaptor at the other end, as illustrated in FIG. 4. The first adaptor can be appended to the nucleic acid fragment by a priming reaction and the second adaptor can subsequently be appended by ligation, as described herein. The oligonucleotide can comprise random sequence at the 3′ portion that is hybridizable to one or more nucleic acid fragments and can further comprise first adaptor sequence at the 5′-portion. Extension of the oligonucleotide along the nucleic acid fragment can generate an oligonucleotide extension product, or products, comprising the first adaptor at a first end. The second adaptor can then be ligated to a second end of the oligonucleotide extension product generating an oligonucleotide extension product comprising a first adaptor sequence at a first end and a second adaptor sequence at a second end. Random sequence present in the oligonucleotide can bind to and be extended on one or more nucleic acid inserts. One or more oligonucleotides comprising a 3′ portion comprising random sequence and a 5′ portion comprising second adaptor sequence can be annealed to a library comprising nucleic acid inserts comprising a first adaptor sequence on one or both ends of each of the nucleic acid inserts. One or more second adaptors can be ligated to a library comprising nucleic acid inserts comprising a first adaptor sequence on one end of each of the nucleic acid inserts. The first adaptor can be the same or common to each of the nucleic acid inserts. The first adaptor sequence can be the same or common to each of the one or more oligonucleotides. The second adaptor can be the same or common to each of the nucleic acid inserts. The second adaptor sequence can be the same or common to each of the one or more oligonucleotides. The methods described above can be used to generate a library of nucleic acid inserts wherein each of the nucleic acid inserts comprises a common first adaptor on one end and a common second adaptor sequence on a second end. The first adaptor and the second adaptor sequence can be distinct from each other. The first adaptor and the second adaptor sequence can comprise the same adaptor sequence. The methods described herein can be used for the high efficiency generation of a library of nucleic acid sequences.

In yet another alternate embodiment to the methods of the invention as described above, the first adaptor can be a double stranded DNA adaptor comprising a partial duplex, wherein the two strands of the adaptor can be different lengths with a complementary region and an overhanging region at the 5′ end. In this embodiment, the 5′ end of the long strand of the partial duplex adaptor can comprise a unique site for a nucleic acid modifying enzyme, such as a restriction enzyme, that is absent from the short strand of the duplex adaptor. In a further embodiment, the 3′ end of the short strand adaptor can be modified by a replacement of the 3′ OH-group by a blocking group, for example, a dideoxynucleotide (ddCMP, ddAMP, ddTMP, or ddGMP) to prevent polymerase extension. In this embodiment, the first adaptor comprising the partial duplex can be ligated to nucleic acid fragments generated by the methods described herein. In one embodiment, ligation of the partial duplex first adaptor can be followed by a gap repair reaction as described above. In this embodiment, ligation of the partial duplex first adaptor is not followed by a gap repair reaction. In a preferred embodiment, the partial duplex first adaptor comprises a free 5′ phosphate on the short strand and a free 3′hydroxyl on the long strand. In this embodiment, ligation of the partial duplex adaptor generates double stranded nucleic acid fragments wherein both ends of the double stranded nucleic acid fragment comprise the long strand and short strand of the partial duplex first adaptor. A double stranded partial duplex first adaptor-nucleic acid fragment complex can be generated by ligation. In one embodiment, the double stranded partial duplex first adaptor-nucleic acid fragment complex can be denatured to generate a single stranded nucleic acid fragment comprising the long strand of the first adaptor on a first end and the short strand of the first adaptor on a second end. In this embodiment, the first end is the 5′ end and the second end is the 3′ end. In one embodiment, the first adaptor can be appended to one or more nucleic acid fragments as generated by the methods described herein such that each of the nucleic acid fragments comprises the same first adaptor or, in other words, the first adaptor can be common to each of the nucleic acid inserts. An oligonucleotide or primer comprising sequence complementary to a sequence of interest in the single stranded nucleic acid fragment can be annealed to the single stranded nucleic acid fragment and extended using a polymerase. In one embodiment, the polymerase can be a DNA dependent DNA polymerase. In one embodiment, the DNA dependent DNA polymerase can be any of the DNA dependent DNA polymerases as described herein and extension of the oligonucleotide can be by any of the methods known in the art. Extension of the primer annealed to the single stranded nucleic acid fragment generates an oligonucleotide extension product comprising sequence complementary to the long strand of the first adaptor on one end. In one embodiment, the oligonucleotide extension product remains hybridized to the single stranded nucleic acid fragment such that the restriction and/or cleavage site specific for a nucleic acid modifying enzyme is made double stranded. The double stranded site can then be cleaved by the nucleic acid modifying enzyme specific for the double stranded restriction site. In one embodiment, the nucleic acid modifying enzyme can be a restriction enzyme. In one embodiment, the restriction enzyme can be specific for a double stranded restriction site. In one embodiment, cleavage of the restriction site can generate a blunt end or non-blunt end. In one embodiment, end repair by any of the methods described herein can be performed on the end of the nucleic acid fragment following cleavage. Cleavage of the restriction and/or cleavage site generates a site to which a second adaptor can be ligated. Ligation of the second adaptor can be through any of the methods for ligation as described herein. In one embodiment, ligation generates a double stranded nucleic acid fragment comprising the second adaptor on a first end and a partial duplex on a second end, wherein the partial duplex comprises a 3′ overhang comprising the sequence of the short strand of the first adaptor. The double stranded nucleic acid fragment can then be denatured using any of the methods for denaturation disclosed herein to generate a single stranded nucleic acid fragment comprising the second adaptor sequence on the first end and the sequence of the short strand of the first adaptor on the second end. In one embodiment, the first end and second end comprise the 5′ end and 3′ end, respectively. In one embodiment, the second adaptor can be appended to one or more nucleic acid fragments following cleavage of the double stranded restriction site such that each of the nucleic acid fragments comprises the same second adaptor or, in other words, the second adaptor can be common to each of the nucleic acid inserts. The single stranded nucleic acid fragment can then be amplified using a first primer specific for the second adaptor and a second primer specific for sequence present in the short strand of the first adaptor. In one embodiment, the amplification reaction can be exponential, and may be carried out at various temperature cycles or isothermal. In one embodiment, the amplification can be polymerase chain reaction. In one embodiment, the amplification reaction can be isothermal. Overall, only a fragment comprising the second adaptor and the short strand of the first adaptor will be amplified or enriched. In so far as the method provides for enrichment of targeted fragments of the library, and not enrichment of oligonucleotide extension products generated by the extension of the oligonucleotide comprising sequence complementary to a target sequence of interest, there is no distortion of the original DNA library, and the enrichment is independent of the insert length. Because the 3′ end of the short strand of the partial duplex adaptor is 3′ blocked, the method enables directional or asymmetric ligation. In one embodiment, the oligonucleotide that comprises sequence complementary to a sequence of interest in a nucleic acid fragment further comprises reverse adaptor sequence. In this embodiment, the sequence complementary to a sequence of interest in the nucleic acid fragment can be present in a 3′ portion of the oligonucleotide and the reverse adaptor sequence can be present at a 5′ portion. Further to this embodiment, the reverse adaptor sequence can be a common or conventional adaptor sequence and can be different or distinct from the first and/or second adaptors. Further still to this embodiment, the methods described above can lead to the generation of a single stranded nucleic acid fragment comprising the second adaptor at one end and the reverse adaptor sequence at the other end. Subsequent to this embodiment, the single stranded nucleic acid fragment can be enriched through amplification using a first primer specific to the second adaptor and a second primer specific to the third adaptor sequence.

The methods of the inventions are further applicable to any enrichment of target nucleic acid sequences of interest from libraries comprising fragments of nucleic acid of a sample appended with adaptor sequence at one or both ends, wherein the libraries are generated using ligation of the adaptor or adaptor sequences to one or both ends as described herein or by ligation independent methods, such as for example Nextera, a transposome driven method. In one embodiment, the nucleic acid can be DNA such as genomic DNA or cDNA. In one embodiment, the nucleic acid can be double stranded. Enrichment of nucleic acid sequences of interest can be achieved using the methods described herein for target enrichment. In one embodiment, the method for enriching for target nucleic acid sequences of interest from a library comprising nucleic inserts with adaptors appended to one or both ends comprises denaturing the nucleic acid inserts to generate a library of single stranded nucleic acid inserts. In one embodiment, each of the nucleic acid inserts can comprise a first adaptor sequence on one end and a second adaptor sequence on an opposite end. In one embodiment, the first adaptor and the second adaptor can be distinct from each other. In one embodiment, the first adaptor and the second adaptor can comprise the same adaptor sequence. In one embodiment, each of the nucleic acid inserts can comprise a first adaptor sequence on one end and a second adaptor sequence on an opposite end such that denaturation generates a library of single stranded nucleic acid inserts comprising the first adaptor sequence on one end and the second adaptor sequence on an opposite end. Denaturation can be achieved using any of the methods described herein. Further to the embodiments described above, one or more oligonucleotides can be annealed to the single stranded nucleic acid inserts. In one embodiment, each of the one or more oligonucleotides comprises a 3′ portion that is complementary to a target nucleic acid sequence of interest present in one or more of the nucleic acid inserts, and a 5′ portion comprising a third adaptor sequence. In one embodiment, the third adaptor sequence is distinct from either or both of the first adaptor and the second adaptor. The one or more oligonucleotides can be extended with a polymerase (e.g., a DNA polymerase) thereby generating one or more oligonucleotide extension products with the first or second adaptor at a first end and the third adaptor sequence at a second end. In one embodiment, the first end comprises the 5′ end and the second end comprises the 3′ end. The one or more oligonucleotide extension products can be amplified using a first primer that can be complementary to the first or second adaptor and a second primer that can be complementary to the third adaptor sequence to enrich for nucleic acid fragments comprising the first or second adaptor and the third adaptor sequence at each end. In one embodiment, the first and second adaptors can be common to each of the nucleic acid inserts in the library. In one embodiment, the third adaptor sequence can be common to each of the one or more oligonucleotides. Overall, the target enrichment methods as described above can be used to generate a composition comprising a library of nucleic acid inserts enriched for any target sequence of interest from a non-enriched library comprised of nucleic acid inserts with an adaptor ligated to one or both ends.

A schematic of a preferred embodiment of the methods described herein for enriching for target sequences of interest is illustrated in FIG. 1. Overall, FIG. 1 depicts a method for isolating or enriching for a nucleic acid fragment or insert comprising a target nucleic acid sequence from a library or plurality of nucleic acid fragments. The method in FIG. 1 involves generation of a ligated library of nucleic acid fragments or inserts wherein each fragment or insert of the ligated library comprises a common forward adaptor and a fragment or insert specific reverse adaptor distinct from the forward adaptor such that subtractive PCR using a primer directed against the common forward adaptor and a primer directed against the reverse adaptor enriches for a nucleic acid fragment or insert comprising a target nucleic acid sequence. The input for the method depicted in FIG. 1 is fragmented DNA. The fragmented DNA is double stranded and comprises a plurality or library of DNA fragments. In one embodiment, the DNA fragments can be derived from complex DNA, such as double-stranded DNA, genomic DNA or mixed DNA from more than one organism. In one embodiment, the DNA fragments can be derived from RNA that has been converted to cDNA through a first strand synthesis reaction using any of the methods well known in the art for generating cDNA from an RNA template which can include, but is not limited to, combining the RNA with a primer (e.g., random primer), and reverse transcribing the RNA template with an RNA-dependent DNA polymerase. In one embodiment, the DNA fragments can be derived from RNA that has been converted to double stranded cDNA through a first and second strand synthesis reaction using any of the methods well known in the art. Fragmentation of the DNA to produce the DNA fragments can be achieved through any of the methods described herein for fragmenting nucleic acids which can include, but are not limited to, physical (e.g., sonication), and/or chemical (e.g., restriction enzyme treatment) fragmentation reactions.

As depicted in FIG. 1, a single forward adaptor is ligated to the DNA fragments. In one embodiment, the single forward adaptor can comprise known sequence. In one embodiment, the single forward adaptor can be a common adaptor. In one embodiment, the DNA fragments can be subjected to an end repair reaction as described herein to produce blunt ends. In this embodiment, the single forward adaptor can also comprise blunt ends and ligation between the single forward adaptor and the DNA fragments can be through blunt end ligation as described herein. Ligation can be facilitated through the use of enzymes (e.g., T4 DNA ligase) and methods known in the art, including, but not limited to, commercially available kits such as the Encore Ultra Low Input NGS Library System. In FIG. 1, the forward adaptor can contain a strand (the ligation strand) that ligates with the free 5′phosphate on a 5′ end of the DNA fragments and a strand that does not ligate (non-ligation strand) to a 3′ end of the DNA fragments. In one embodiment, the ligation reaction can lead to the generation of a nick or gap between the non-ligation strand of the single forward adaptor and the 3′ end of the DNA fragments. In this embodiment, the nick or gap can be repaired or filled in through a gap repair or fill-in reaction wherein the 3′ end of the DNA fragments can be extended with a polymerase (preferably with a DNA dependent DNA polymerase such as Taq DNA polymerase) wherein the ligation strand of the forward adaptor can serve as template. In this embodiment, the gap repair generates DNA fragments with complementary ends. As depicted in FIG. 1, the DNA fragments with complementary ends are denatured to generate a denatured library comprising single stranded DNA fragments with complementary ends. Denaturation can be achieved using any of the methods known in the art which can include, but are not limited to, heat denaturation, and/or chemical denaturation.

As depicted in FIG. 1, a custom oligonucleotide with a reverse adaptor tail is annealed to the single stranded DNA fragments with complementary ends. In one embodiment, the custom oligonucleotide with a reverse adaptor tail can comprise a 3′ portion comprising sequence complementary to a target sequence of interest in one of the single-stranded DNA fragments and a 5′ portion comprising reverse adaptor sequence that is not complementary to the single-stranded DNA fragments in the denatured library. In one embodiment, the reverse adaptor sequence can be known sequence. In a preferred embodiment, the reverse adaptor sequence can be distinct from the single forward adaptor as described herein. In one embodiment, a plurality of custom oligonucleotides with a reverse adaptor tail can be added to the denatured library wherein the plurality of custom oligonucleotides with a reverse adaptor tail comprise a 3′ portion comprising sequence complementary to a target sequence of interest in one or more of the single-stranded DNA fragments of the denatured library and a 5′ portion comprising a reverse adaptor sequence that is not complementary to the single-stranded DNA fragments in the denatured library. In one embodiment, the reverse adaptor tail comprises the same reverse adaptor sequence in each of the plurality of custom oligonucleotides with a reverse adaptor tail, and wherein the reverse adaptor sequence is distinct from the forward adaptor sequence. In another embodiment, the reverse adaptor tail comprises a different reverse adaptor sequence for each of the plurality of custom oligonucleotides with a reverse adaptor tail, and wherein each of the different reverse adaptor sequences is distinct from the forward adaptor sequence. In one embodiment, the 3′ portion of the custom oligonucleotide with a reverse adaptor tail can be a specific sequence, wherein the custom oligonucleotide comprises a sequence complementary to the target sequence of interest and provides a means for targeted enrichment of sequence or sequences of interest using the methods of the invention. In another embodiment the 3′ portion of the custom oligonucleotide with reverse adaptor tail can be a randomly generated sequence hybridizable to random sequences of the library of fragments with adaptor sequences on one or both ends, providing means for efficient, non-enriched library generation employing the methods of the invention.

Following annealing of the custom oligonucleotide with a reverse adaptor tail to a sequence of interest in a single-stranded DNA fragment of the denatured library, the custom oligonucleotide with a reverse adaptor tail is extended using any method known in the art, which can include but is not limited to, extension using a DNA dependent DNA polymerase using the single stranded DNA fragment of the denatured library as a template. Extension of the custom oligonucleotide with a reverse adaptor tail generates an oligonucleotide extension product with forward adaptor sequence at one end and reverse adaptor sequence at the other end. In this embodiment, the custom oligonucleotide with a reverse adaptor tail can only anneal to and be extended on DNA fragments in the denatured library comprising the target sequence of interest for which the custom oligonucleotide with a reverse adaptor tail is directed. As illustrated in FIG. 1, a subtractive polymerase chain reaction (PCR) procedure is subsequently performed using a first primer directed against the forward adaptor sequence and a second primer directed toward the reverse adaptor sequence such that only the oligonucleotide extension product with the forward adaptor sequence at one end and the reverse adaptor sequence at the other end can be amplified and thus enriched.

FIG. 2 depicts another embodiment of subtractive PCR enrichment method as described for FIG. 1, wherein ligation of a duplex forward adaptor (P1) to a double stranded nucleic acid fragment is not subjected to gap repair. The duplex forward adaptor (P1) comprises a strand (the ligation strand; P1) that ligates with the free 5′phosphate on a 5′ end of the nucleic acid fragment and a strand that does not ligate (non-ligation strand; P1rc) to a 3′ end of the nucleic acid fragment. In this embodiment, the ligation strand is ligated to the 5′ end of both strands of a double stranded nucleic fragment whereas a gap or nick is generated between the non-ligation strand and the 3′end of both strands of the double stranded nucleic acid fragment. As depicted in FIG. 2, the ligation of the P1 adaptor to the nucleic acid fragment is followed by denaturation without a gap repair or fill-in reaction thereby generating a single stranded nucleic acid fragment with non-complementary ends. In this embodiment, the single stranded nucleic acid fragment with non-complementary ends comprises a P1 forward adaptor sequence at a 5′ end and fragment specific sequence at a 3′ end. In a further embodiment, the single stranded nucleic fragment with non-complementary ends can be further processed as described above and illustrated in FIG. 1 to generate a single stranded nucleic fragment with the P1 forward adaptor sequence at the 5′ end and a distinct reverse adaptor sequence on the 3′ end.

FIG. 4 illustrates another embodiment of the present invention for the high efficiency generation of libraries comprising nucleic acid fragments with distinct adaptors on each end. In this embodiment, the methods for generating a denatured library comprising single stranded DNA fragments with a single forward adaptor sequence on a 5′ end and a reverse adaptor sequence on a 3′ end is similar to the method described for FIG. 1. However, in FIG. 4, an oligonucleotide with a reverse adaptor tail is used whereby the oligonucleotide comprises a 3′ portion comprising random sequence and a reverse adaptor tail wherein the reverse adaptor tail comprises reverse adaptor sequence that is distinct from the single forward adaptor sequence. As illustrated in FIG. 4, PCR can be carried out with a first primer directed against the single forward adaptor sequence and a second primer directed toward the reverse adaptor sequence wherein both the first and second primers further comprise flow cell sequences. In this manner, single stranded DNA fragments with a single forward adaptor sequence on a 5′ end and a reverse adaptor sequence on a 3′ end comprise flow cell sequences that can be used to adhere the amplified single stranded DNA fragments to flow cells for subsequent sequencing by the method commercialized by Illumina, as described U.S. Pat. Nos. 5,750,341; 6,306,597; and 5,969,119.

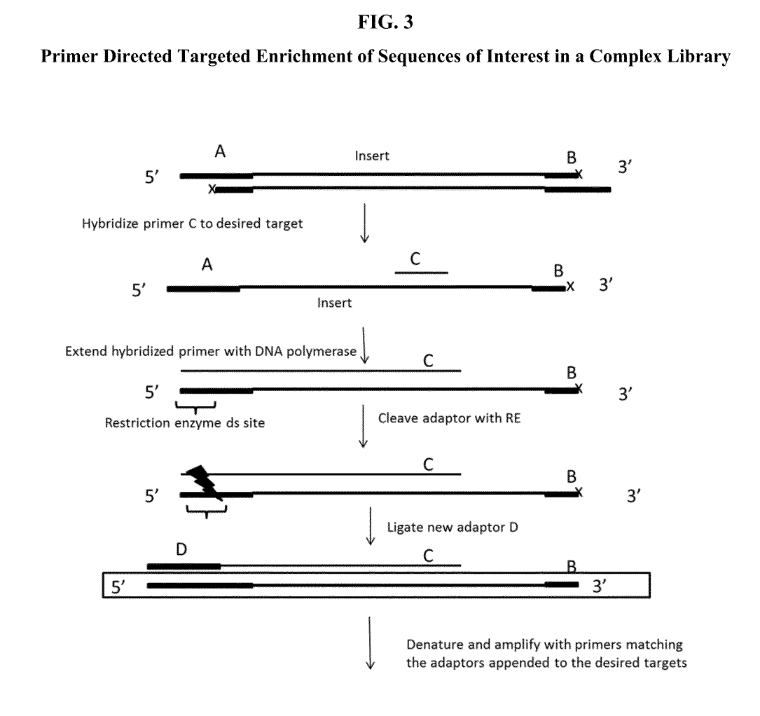

FIG. 3 illustrates a method for enrichment of a target nucleic acid sequence, or sequences of interest, contained in a double stranded nucleic acid insert from a complex library. In one embodiment, the complex library comprises nucleic acid inserts from a genomic DNA sample, In FIG. 3, the single forward adaptor comprises a partial duplex forward adaptor comprising a long strand, A, that forms a partial duplex with a short strand, B. Strand A of the partial duplex adaptor further comprises a restriction enzyme site while strand B does not contain the restriction enzyme site. Strand B further comprises a blocking group wherein the 3′ end of strand B is modified by replacement of the 3′ OH group with a blocking group that can prevent polymerase extension. In one embodiment, the partial duplex forward adaptor is ligated to the double stranded nucleic acid fragments such that a double stranded insert with the partial duplex forward adaptor appended to both ends is generated. In this embodiment, the 5′ end of strand B of the partial duplex adaptor can contain a free 5′ phosphate which can be ligated to a free 3′ OH present on one or both strands of the double stranded insert. Subsequent denaturation generates a single stranded insert comprising sequence A on a 5′ end and strand B on a 3′ end. A primer, C, directed against a specific sequence of interest within the single stranded insert can be annealed to the specific sequence and extended with a DNA polymerase using the single stranded insert as a template. In one embodiment, primer C can be a sequence specific primer and is employed for enrichment of target, or targets, of interest according to the methods of the invention. In one embodiment, primer C can be a random primer. Extension of primer C with DNA polymerase generates an extended primer C product that comprises sequence complementary to sequence A at its' 3′ end in a double stranded complex with the template insert strand such that a double stranded restriction site has been generated between sequence A and its' complement. In one embodiment, the double stranded restriction enzyme recognition site can be cleaved by a restriction enzyme specific for the double stranded restriction site thereby generating a truncated, or cleaved, adaptor sequence at one end. A second forward adaptor comprising a common, or conventional, duplex adaptor D is then ligated to the cleavage site using any of the ligation methods described herein, thereby generating a double stranded complex comprising the second forward adaptor D at one end and a 3′ overhang comprising strand B on the opposite end. The double stranded complex comprising the second forward adaptor D at one end and a 3′ overhang comprising strand B on the opposite end is denatured and amplified using a first primer directed against the second forward adaptor D and a second primer directed against strand B. In this manner, the methods depicted in FIG. 3 can be used to enrich for specific sequences of interest from a complex library since the methods are designed such that the second forward adaptor D can only bind to the double stranded cleavage site generated by restriction enzyme digestion of the double stranded restriction site created between sequence A and its complement following extension of primer C. As described, primer C can be directed against a target sequence of interest present in a single, or multiple insert, or inserts, amongst a plurality of inserts. Moreover, the method can be made strand specific by designing primer C to bind to target sequence of interest present on one strand or the other of an insert in an amongst a plurality of inserts.

Unless otherwise specified, terms and symbols of genetics, molecular biology, biochemistry and nucleic acid used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

### Input Nucleic Acid

The input can be a nucleic acid. In one embodiment, the input can be DNA. In one embodiment, the input nucleic acid can be complex DNA, such as double-stranded DNA, genomic DNA or mixed DNA from more than one organism. In one embodiment, the input can be RNA. In one embodiment, the RNA can be obtained and purified using standard techniques in the art and include RNAs in purified or unpurified form, which include, but are not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA may include snoRNAs, microRNAs, siRNAs, piRNAs and long nc RNAs. In one embodiment, the input nucleic acid can be cDNA. The cDNA can be generated from RNA, e.g., mRNA. The cDNA can be single or double stranded. The input DNA can be of a specific species, for example, human, rat, mouse, other animals, specific plants, bacteria, algae, viruses, and the like. The input complex also can be from a mixture of genomes of different species such as host-pathogen, bacterial populations and the like. The input DNA can be cDNA made from a mixture of genomes of different species. Alternatively, the input nucleic acid can be from a synthetic source. The input DNA can be mitochondrial DNA. The input DNA can be cell-free DNA. The cell-free DNA can be obtained from, e.g., a serum or plasma sample. The input DNA can comprise one or more chromosomes. For example, if the input DNA is from a human, the DNA can comprise one or more of chromosome 1, 2, 3, 4, 5, 6, 7, 8, 9, 10, 11, 12, 13, 14, 15, 16, 17, 18, 19, 20, 21, 22, X, or Y. The DNA can be from a linear or circular genome. The DNA can be plasmid DNA, cosmid DNA, bacterial artificial chromosome (BAC), or yeast artificial chromosome (YAC). The input DNA can be from more than one individual or organism. The input DNA can be double stranded or single stranded. The input DNA can be part of chromatin. The input DNA can be associated with histones.

In some embodiments, the oligonucleotides targeting the selected sequence regions of interest are designed to hybridize to single-stranded nucleic acid targets. In one embodiment, the oligonucleotides targeting the selected sequence regions of interest are designed to hybridize to single-stranded DNA targets. In the case where the input nucleic acid sample comprises genomic DNA or other double-stranded DNA, the input nucleic acid sample can be first denatured to render the target single stranded and enable hybridization of the oligonucleotides to the desired sequence regions of interest. In these embodiments, the methods and compositions described herein can allow for region-specific enrichment and amplification of sequence regions of interest. In some embodiments, the other double-stranded DNA can be double-stranded cDNA generated by first and second strand synthesis of one or more target RNAs.

In other embodiments, the oligonucleotides targeting the selected sequence regions of interest are designed to hybridize to double-stranded nucleic acid targets, without denaturation of the double stranded nucleic acids. In other embodiments, the oligonucleotides targeting the selected sequence regions of interest are designed to hybridize to a double-stranded DNA target, without denaturation of the dsDNA. In these embodiments, the oligonucleotides targeting the selected sequence regions of interest are designed to form a triple helix (triplex) at the selected sequence regions of interest. The hybridization of the oligonucleotides to the double-stranded DNA sequence regions of interest can be carried out without prior denaturation of the double stranded nucleic acid sample. In such embodiments, the methods and compositions described herein can allow for region-specific enrichment as well as strand-specific enrichment and amplification of sequence regions of interest. This method can be useful for generation of copies of strand specific sequence regions of interest from complex nucleic acid without the need to denature the dsDNA input DNA, thus enabling enrichment and analysis of multiplicity of sequence regions of interest in the native complex nucleic acid sample. The method can find use for studies and analyses carried out in situ, enable studies and analysis of complex genomic DNA in single cells or collection of very small well defined cell population, as well as permit the analysis of complex genomic DNA without disruption of chromatin structures.

A “target nucleic acid sequence” or “target sequence” as used herein, is a polynucleotide sequence of interest, for which enrichment is desired. The target sequence may be known or not known, in terms of its actual sequence. Generally, a “template”, as used herein, is a polynucleotide that contains the target nucleic acid sequence. The terms “target sequence,” “target nucleic acid sequence,” “target nucleotide sequence,” “regions of interest,” or “sequence of interest” and, variations thereof, are used interchangeably.

### Oligonucleotides of the Invention

As used within the invention, the term “oligonucleotide” refers to a polynucleotide chain, typically less than 200 residues long, most typically between 15 and 100 nucleotides long, but also intended to encompass longer polynucleotide chains. Oligonucleotides may be single- or double-stranded. As used in this invention, the term “oligonucleotide” may be used interchangeably with the terms “primer” and “adaptor”.

As used herein, the terms “hybridization”/“hybridizing” and “annealing” are used interchangeably and refer to the pairing of complementary nucleic acids.

The term “primer”, as used herein, can refer to a nucleotide sequence, generally with a free 3′ hydroxyl group, that is capable of hybridizing with a template (such as one or more target polynucleotides, one or more target DNAs, one or more target RNAs or a primer extension product) and is also capable of promoting polymerization of a polynucleotide complementary to the template. A primer can be, for example, an oligonucleotide. It can also be, for example, a sequence of the template (such as a primer extension product or a fragment of the template created following RNase (e.g., RNase H) cleavage of a template-DNA complex) that is hybridized to a sequence in the template itself (for example, as a hairpin loop), and that is capable of promoting nucleotide polymerization. Thus, a primer can be an exogenous (e.g., added) primer or an endogenous (e.g., template fragment) primer. A primer may contain a non-hybridizing sequence that constitutes a tail of the primer. A primer may still be hybridizing to a target even though its sequences are not fully complementary to the target.

The primers of the invention are generally oligonucleotides that are employed in an extension reaction by a polymerase along a polynucleotide template, such as in PCR, SPIA or cDNA synthesis, for example. The oligonucleotide primer can be a synthetic polynucleotide that is single stranded, containing a sequence at its 3′-end that is capable of hybridizing with a sequence of the target polynucleotide. Normally, the 3′ region of the primer that hybridizes with the target nucleic acid has at least 80%, preferably 90%, more preferably 95%, most preferably 100%, complementarity to a sequence or primer binding site.

“Complementary”, as used herein, can refer to complementarity to all or only to a portion of a sequence. The number of nucleotides in the hybridizable sequence of a specific oligonucleotide primer should be such that stringency conditions used to hybridize the oligonucleotide primer will prevent excessive random non-specific hybridization. Usually, the number of nucleotides in the hybridizing portion of the oligonucleotide primer will be at least as great as the defined sequence on the target polynucleotide that the oligonucleotide primer hybridizes to, namely, at least 5, at least 6, at least 7, at least 8, at least 9, at least 10, at least 11, at least 12, at least 13, at least 14, at least 15, at least about 20, and generally from about 6 to about 10 or 6 to about 12 or 12 to about 200 nucleotides, usually about 20 to about 50 nucleotides. In general, the target polynucleotide is larger than the oligonucleotide primer or primers as described previously.

In some cases, the identity of the investigated target polynucleotide sequence is known, and hybridizable sequence specific oligonucleotides or primers can be synthesized precisely according to the antisense sequence of the aforesaid target polynucleotide sequence. In some embodiments, multiple sequence-specific oligonucleotides or primers are employed to hybridize to a multiplicity of genomic regions of interest, allowing for selective enrichment of the regions of interest. In so far as the genomic regions may be very long, multiple oligonucleotides can be designed to hybridize to different sequence regions within the genomic regions of interest. In other embodiments, when the target polynucleotide sequence is unknown, the hybridizable sequence of an oligonucleotide or primer is a random sequence. Oligonucleotides or primers comprising random sequences may be referred to as “random primers”, or “random oligonucleotides,” as described herein. In one embodiment, an oligonucleotide or primer of the present invention hybridizable to a target sequence may comprise a mixture of primers or oligonucleotides designed to hybridize to a plurality (e.g. 2, 3, 4, about 6, 8, 10, 20, 40, 80, 100, 125, 150, 200, 250, 300, 400, 500, 600, 800, 1000, 1500, 2000, 2500, 3000, 4000, 5000, 6000, 7000, 8000, 10,000, 20,000, 25,000 or more) of target sequences. In some cases, the plurality of target sequences may comprise a group of related sequences, random sequences, a whole transcriptome or fraction (e.g. substantial fraction) thereof, or any group of sequences such as mRNA. In some embodiments, the primers can be directed to known sequences present in the adaptors used in the invention as described herein. In this embodiment, the primers can comprise groups of primers comprising one or more primers in each group, wherein each group of primers can be directed against distinct adaptors.

Tailed primers or oligonucleotides can be employed in certain embodiments of the invention. In general, a tailed primer comprises a 3′ portion that is hybridizable to one or more target polynucleotides, and a 5′ portion that is not hybridizable to the one or more target polynucleotides. In general, the non-hybridizable 5′ portion does not hybridize to the one or more target polynucleotides under conditions in which the hybridizable 3′ portion of the tailed primer hybridizes to the one or more target polynucleotides. In some embodiments, the non-hybridizable 5′ portion comprises an adaptor sequence. In some embodiments, the non-hybridizable 5′ portion comprises a common or conventional adaptor sequence. In some embodiments, the non-hybridizable 5′ portion comprises a common or conventional adaptor sequence that is distinct or different from the sequence of other adaptors used in the present invention. In some embodiments, the non-hybridizable 5′ portion comprises a promoter-specific sequence. Generally, a promoter-specific sequence comprises a single-stranded DNA sequence region which, in double-stranded form is capable of mediating RNA transcription. Examples of promoter-specific sequences are known in the art, and include, without limitation, T7, T3, or SP6 RNA polymerase promoter sequences. When the tailed primer is extended with a DNA polymerase, a primer extension product with a 5′ portion comprising a defined sequence can be created. This primer extension product can then have a second primer anneal to it, which can be extended with a DNA polymerase to create a double stranded product comprising a defined sequence at one end. In some embodiments, where the non-hybridizable 5′ portion of one or more tailed primers comprises a promoter-specific sequence, creation of a double-stranded product comprising a defined sequence at one end generates a double-stranded promoter sequence that is capable of mediating RNA transcription. In some embodiments, a double-stranded promoter sequence can be generated by hybridizing to the promoter-specific sequence an oligonucleotide comprising a sequence complementary to the promoter-specific sequence. In some embodiments, formation of a double-stranded promoter can be followed by the generation of single-stranded RNA by RNA transcription of sequence downstream of the double-stranded promoter, generally in a reaction mixture comprising all necessary components, including but not limited to ribonucleoside triphosphates (rNTPs) and a DNA-dependent RNA polymerase. Tailed primers can comprise DNA, RNA, or both DNA and RNA. In some embodiments, the tailed primer consists of DNA.

Composite primers can be employed in certain embodiments of the invention. Composite primers are primers that are composed of RNA and DNA portions. In some aspects, the composite primer can be a tailed composite primer comprising, for example, a 3′-DNA portion and a 5′-RNA portion. In the tailed composite primer, a 3′-portion, all or a portion of which comprises DNA, is complementary to a polynucleotide; and a 5′-portion, all or a portion of which comprises RNA, is not complementary to the polynucleotide and does not hybridize to the polynucleotide under conditions in which the 3′-portion of the tailed composite primer hybridizes to the polynucleotide target. When the tailed composite primer is extended with a DNA polymerase, a primer extension product with a 5′-RNA portion comprising a defined sequence can be created. This primer extension product can then have a second primer anneal to it, which can be extended with a DNA polymerase to create a double stranded product with an RNA/DNA heteroduplex comprising a defined sequence at one end. The RNA portion can be selectively cleaved from the partial heteroduplex to create a double-stranded DNA with a 3′-single-stranded overhang which can be useful for various aspects of the present invention including allowing for isothermal amplification using a composite amplification primer.

A “random primer,” as used herein, can be a primer that generally comprises a sequence that is designed not necessarily based on a particular or specific sequence in a sample, but rather is based on a statistical expectation (or an empirical observation) that the sequence of the random primer is hybridizable (under a given set of conditions) to one or more sequences in the sample. A random primer will generally be an oligonucleotide or a population of oligonucleotides comprising a random sequence(s) in which the nucleotides at a given position on the oligonucleotide can be any of the four nucleotides, or any of a selected group of the four nucleotides (for example only three of the four nucleotides, or only two of the four nucleotides). In some cases all of the positions of the oligonucleotide or population of oligonucleotides can be any of two or more nucleotides. In other cases, only a portion of the oligonucleotide, for instance a particular region, will comprise positions which can be any of two or more bases. In some cases, the portion of the oligonucleotide which comprises positions which can be any of two or more bases is about 5, 6, 7, 8, 9, 10, 11, 12, 13, 14, or about 15-20 nucleotides in length. In some cases, a random primer may comprise a tailed primer having a 3′-region that comprises a random sequence and a 5′-region that is a non-hybridizing sequence that comprises a specific, non-random sequence. The 3′-region may also comprise a random sequence in combination with a region that comprises poly-T sequences. The sequence of a random primer (or its complement) may or may not be naturally-occurring, or may or may not be present in a pool of sequences in a sample of interest. As is well understood in the art, a “random primer” can also refer to a primer that is a member of a population of primers (a plurality of random primers) which collectively are designed to hybridize to a desired and/or a significant number of target sequences. A random primer may hybridize at a plurality of sites on a nucleic acid sequence. The use of random primers provides a method for generating primer extension products complementary to a target polynucleotide or target nucleic sequence which does not require prior knowledge of the exact sequence of the target. In some embodiments one portion of a primer is random, and another portion of the primer comprises a defined sequence. For example, in some embodiments, a 3′-portion of the primer will comprise a random sequence, while the 5′-portion of the primer comprises a defined sequence. In some embodiments a 3′-random portion of the primer will comprise DNA, and a 5′-defined portion of the primer will comprise RNA, in other embodiments, both the 3′ and 5′-portions will comprise DNA. In some embodiments, the 5′-portion will contain a defined sequence and the 3′-portion will comprise a poly-dT sequence that is hybridizable to a multiplicity of RNAs in a sample (such as all mRNA). In some embodiments, a “random primer,” or primer comprising a randomly generated sequence, comprises a collection of primers comprising one or more nucleotides selected at random from two or more different nucleotides, such that all possible sequence combinations of the nucleotides selected at random may be represented in the collection. In some embodiments, generation of one or more random primers does not include a step of excluding or selecting certain sequences or nucleotide combinations from the possible sequence combinations in the random portion of the one or more random primers.

In one embodiment, the oligonucleotides of the invention can be tailed oligonucleotides. In one embodiment, the 5′-tail can comprise RNA and is non hybridizable to the RNA in the sample. In one embodiment, the 5′-tail can comprise DNA and is non hybridizable to the DNA in the sample. In one embodiment, the 5′-tail can comprise an adaptor that is not hydridizable to the DNA and/or nucleic acid fragments derived from the sample comprising nucleic acid. In one embodiment, the 5′-tail can comprise an adaptor sequence that is not hydridizable to the DNA and/or nucleic acid fragments derived from the sample comprising nucleic acid. In some embodiments, the 5′-tail can comprise a common adaptor sequence that is not hydridizable to the DNA and is distinct from any other adaptor or adaptor sequence used in the methods of the invention described herein. In some embodiments, the 5′-tail can comprise an identifier sequence. In some embodiments, the identifier sequence can comprise a barcode sequence. In some embodiments, the 5′-tail can comprise a common adaptor sequence that is not hydridizable to the DNA and a barcode sequence.

The term “adaptor”, as used herein, refers to an oligonucleotide of known sequence, the ligation of which to a target polynucleotide or a target polynucleotide strand of interest enables the generation of amplification-ready products of the target polynucleotide or the target polynucleotide strand of interest. The target polynucleotide molecules may be fragmented or not prior to the addition of adaptors.

Various adaptor designs are envisioned which are suitable for generation of amplification-ready products of target sequence regions/strands of interest. For example, the two strands of the adaptor may be self-complementary, non-complementary or partially complementary. A common feature of the adaptors depicted in FIG. 3 of the invention is the partial duplex design, wherein the two strands of the adaptor are different lengths with a complementary region and an overhanging region at the 5′ end. The 5′ end of the long strand of the partial duplex adaptor contains a unique site for a nucleic acid modifying enzyme, such as a restriction enzyme, that is absent from the short strand of the duplex adaptor. The 3′ end of the short strand adaptor is modified by a replacement of the 3′ OH-group is by a blocking group, for example, a dideoxynucleotide (ddCMP, ddAMP, ddTMP, or ddGMP) to prevent polymerase extension.

In some embodiments of the invention, the adaptors comprise an additional identifier sequence, e.g., a barcode sequence. As used herein, the term “barcode” refers to a known nucleic acid sequence that allows some feature of a polynucleotide with which the barcode is associated to be identified. In some embodiments, the feature of the polynucleotide to be identified is the sample from which the polynucleotide is derived. In some embodiments, barcodes are at least 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, each barcode in a plurality of barcodes differ from every other barcode in the plurality at at least three nucleotide positions, such as at least 3, 4, 5, 6, 7, 8, 9, 10, or more positions. In some embodiments, barcodes associated with some polynucleotides are of different length than barcodes associated with other polynucleotides. In general, barcodes are of sufficient length and comprise sequences that are sufficiently different to allow the identification of samples based on barcodes with which they are associated. In some embodiments, both the forward and reverse adapter comprise at least one of a plurality of barcode sequences. In some embodiments, the first, second, and/or third adaptor comprises at least one of a plurality of barcode sequences. In some embodiments, each reverse adapter comprises at least one of a plurality of barcode sequences, wherein each barcode sequence of the plurality of barcode sequences differs from every other barcode sequence in the plurality of barcode sequences. In some embodiments, both the first adapter and the second adapter comprise at least one of a plurality of barcode sequences. In some embodiments, barcodes for second adapter oligonucleotides are selected independently from barcodes for first adapter oligonucleotides. In some embodiments, first adapter oligonucleotides and second adapter oligonucleotides having barcodes are paired, such that adapters of the pair comprise the same or different one or more barcodes. In some embodiments, the methods of the invention further comprise identifying the sample from which a target polynucleotide is derived based on the barcode sequence to which the target polynucleotide is joined. In general, a barcode comprises a nucleic acid sequence that when joined to a target polynucleotide serves as an identifier of the sample from which the target polynucleotide was derived.

Recently, many improvements have been made in adaptor design that have reduced the occurrence of adapter dimer. These improvements include the use of nucleotide analogs and structured oligonucleotides, and have allowed for use of higher concentrations of oligonucleotides in ligation reactions. The higher concentrations of adapters in ligation reactions have enabled researchers to produce high quality libraries from as few as 150 copies of genome. Ligation of adaptors to the ends of DNA fragments, in particular those fragments containing the regions of interest is suitable for carrying out the methods of the invention. Various ligation modalities are envisioned, dependent on the choice of nucleic acid modifying enzymes and the resulting double-stranded DNA cleavage. For example, when a blunt end product comprising the target region/sequence of interest is generated, blunt end ligation can be suitable. Alternatively, where the cleavage is carried out using a restriction enzyme of known sequence specificity, leading to the generation of cleavage sites with known sequence overhangs, suitable ends of the adaptors can be designed to enable hybridization of the adaptor to the cleavage site of the sequence region of interest and subsequent ligation. Reagents and methods for efficient and rapid ligation of adaptors are commercially available and are known in the art.

### Nucleic Acid Modifying Enzymes

The nucleic acid (NA)-modifying enzyme can be DNA-specific modifying enzyme. The NA-modifying enzyme can be selected for specificity for double-stranded DNA. The enzyme can be a duplex-specific endonuclease, a blunt-end frequent cutter restriction enzyme, or other restriction enzyme. Examples of blunt-end cutters include DraI or SmaI. The NA-modifying enzyme can be an enzyme provided by New England Biolabs. The NA-modifying enzyme can be a homing endonuclease (a homing endonuclease can be an endonuclease that does not have a stringently-defined recognition sequence). The NA-modifying enzyme can be a nicking endonuclease (a nicking endonuclease can be an endonuclease that can cleave only one strand of DNA in a double-stranded DNA substrate). The NA-modifying enzyme can be a high fidelity endonuclease (a high fidelity endonuclease can be an engineered endonuclease that has less “star activity” than the wild-type version of the endonuclease).

In a preferred embodiment, the NA-modifying enzyme is a sequence and duplex-specific, DNA modifying enzyme.

### DNA Dependent DNA Polymerases

DNA-dependent DNA polymerases for use in the methods and compositions of the invention are capable of effecting extension of a primer or oligonucleotide according to the methods of the invention. In one embodiment, a preferred DNA-dependent DNA polymerase can be one that is capable of extending a nucleic acid primer in the presence of the DNA and/or cDNA template. Exemplary DNA dependent DNA polymerases suitable for the methods of the present invention include but are not limited to Klenow polymerase, with or without 3′-exonuclease, Bst DNA polymerase, Bca polymerase, .phi.29 DNA polymerase, Vent polymerase, Deep Vent polymerase, Taq polymerase, T4 polymerase, and E. coli DNA polymerase 1, derivatives thereof, or mixture of polymerases. In some cases, the polymerase does not comprise a 5′-exonuclease activity. In other cases, the polymerase comprises 5′ exonuclease activity. In some cases, the primer or oligonucleotide extension of the present invention may be performed using a polymerase comprising strong strand displacement activity such as for example Bst polymerase. In other cases, the primer extension of the present invention may be performed using a polymerase comprising weak or no strand displacement activity. One skilled in the art may recognize the advantages and disadvantages of the use of strand displacement activity during the primer extension step, and which polymerases may be expected to provide strand displacement activity (see e.g., New England Biolabs Polymerases).

### Methods of Amplification

The methods, compositions and kits described herein can be useful to generate amplification-ready products for downstream applications such as massively parallel sequencing (e.g., next generation sequencing methods), generation of libraries with enriched population of sequence regions of interest, or hybridization platforms. Methods of amplification are well known in the art. Suitable amplification reactions can be exponential or isothermal and can include any DNA amplification reaction, including but not limited to polymerase chain reaction (PCR), strand displacement amplification (SDA), linear amplification, multiple displacement amplification (MDA), rolling circle amplification (RCA), single primer isothermal amplification (SPIA, see e.g. U.S. Pat. No. 6,251,639), Ribo-SPIA, or a combination thereof. In some cases, the amplification methods for providing the template nucleic acid may be performed under limiting conditions such that only a few rounds of amplification (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.), such as for example as is commonly done for cDNA generation. The number of rounds of amplification can be about 1-30, 1-20, 1-15, 1-10, 5-30, 10-30, 15-30, 20-30, 10-30, 15-30, 20-30, or 25-30.

PCR is an in vitro amplification procedure based on repeated cycles of denaturation, oligonucleotide primer annealing, and primer extension by thermophilic template dependent polynucleotide polymerase, resulting in the exponential increase in copies of the desired sequence of the polynucleotide analyte flanked by the primers. The two different PCR primers, which anneal to opposite strands of the DNA, are positioned so that the polymerase catalyzed extension product of one primer can serve as a template strand for the other, leading to the accumulation of a discrete double stranded fragment whose length is defined by the distance between the 5′ ends of the oligonucleotide primers.

LCR uses a ligase enzyme to join pairs of preformed nucleic acid probes. The probes hybridize with each complementary strand of the nucleic acid analyte, if present, and ligase is employed to bind each pair of probes together resulting in two templates that can serve in the next cycle to reiterate the particular nucleic acid sequence.

SDA (Westin et al 2000, Nature Biotechnology, 18, 199-202; Walker et al 1992, Nucleic Acids Research, 20, 7, 1691-1696), is an isothermal amplification technique based upon the ability of a restriction endonuclease such as HincII or BsoBI to nick the unmodified strand of a hemiphosphorothioate form of its recognition site, and the ability of an exonuclease deficient DNA polymerase such as Klenow exo minus polymerase, or Bst polymerase, to extend the 3′-end at the nick and displace the downstream DNA strand. Exponential amplification results from coupling sense and antisense reactions in which strands displaced from a sense reaction serve as targets for an antisense reaction and vice versa.

Some aspects of the invention utilize linear amplification of nucleic acids or polynucleotides. Linear amplification generally refers to a method that involves the formation of one or more copies of the complement of only one strand of a nucleic acid or polynucleotide molecule, usually a nucleic acid or polynucleotide analyte. Thus, the primary difference between linear amplification and exponential amplification is that in the latter process, the product serves as substrate for the formation of more product, whereas in the former process the starting sequence is the substrate for the formation of product but the product of the reaction, i.e., the replication of the starting template, is not a substrate for generation of products. In linear amplification the amount of product formed increases as a linear function of time as opposed to exponential amplification where the amount of product formed is an exponential function of time.

In some embodiments, the amplification is exponential, e.g. in the enzymatic amplification of specific double stranded sequences of DNA by a polymerase chain reaction (PCR). In other embodiments the amplification method is linear. In other embodiments the amplification method is isothermal.

### Downstream Applications

An important aspect of the invention is that the methods and compositions disclosed herein can be efficiently and cost-effectively utilized for downstream analyses, such as next generation sequencing or hybridization platforms, with minimal loss of biological material of interest. The methods of the present invention can also be used in the analysis of genetic information of selective genomic regions of interest (e.g., analysis of SNPs or other disease markers) as well as genomic regions which may interact with the selective region of interest.

### Sequencing

For example, the methods of the invention are useful for sequencing by the method commercialized by Illumina, as described U.S. Pat. Nos. 5,750,341; 6,306,597; and 5,969,119. In general, double stranded fragment polynucleotides can be prepared by the methods of the present invention to produce amplified nucleic acid sequences tagged at one (e.g., (A)/(A′) or both ends (e.g., (A)/(A′) and (C)/(C′)). In some cases, single stranded nucleic acid tagged at one or both ends is amplified by the methods of the present invention (e.g., by SPIA or linear PCR). The resulting nucleic acid is then denatured and the single-stranded amplified polynucleotides are randomly attached to the inside surface of flow-cell channels. Unlabeled nucleotides are added to initiate solid-phase bridge amplification to produce dense clusters of double-stranded DNA. To initiate the first base sequencing cycle, four labeled reversible terminators, primers, and DNA polymerase are added. After laser excitation, fluorescence from each cluster on the flow cell is imaged. The identity of the first base for each cluster is then recorded. Cycles of sequencing are performed to determine the fragment sequence one base at a time.

In some embodiments, the methods of the invention are useful for preparing target polynucleotides for sequencing by the sequencing by ligation methods commercialized by Applied Biosystems (e.g., SOLiD sequencing). In other embodiments, the methods are useful for preparing target polynucleotides for sequencing by synthesis using the methods commercialized by 454/Roche Life Sciences, including but not limited to the methods and apparatus described in Margulies et al., Nature (2005) 437:376-380 (2005); and U.S. Pat. Nos. 7,244,559; 7,335,762; 7,211,390; 7,244,567; 7,264,929; and 7,323,305. In other embodiments, the methods are useful for preparing target polynucleotide(s) for sequencing by the methods commercialized by Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058. In other embodiments, the methods are useful for preparing target polynucleotide(s) for sequencing by the methods commercialized by Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764.

Another example of a sequencing technique that can be used in the methods of the provided invention is nanopore sequencing (see e.g. Soni G V and Meller A. (2007) Clin Chem 53: 1996-2001). A nanopore can be a small hole of the order of 1 nanometer in diameter Immersion of a nanopore in a conducting fluid and application of a potential across it can result in a slight electrical current due to conduction of ions through the nanopore. The amount of current that flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore can represent a reading of the DNA sequence.

Another example of a sequencing technique that can be used in the methods of the provided invention is semiconductor sequencing provided by Ion Torrent (e.g., using the Ion Personal Genome Machine (PGM)). Ion Torrent technology can use a semiconductor chip with multiple layers, e.g., a layer with micro-machined wells, an ion-sensitive layer, and an ion sensor layer. Nucleic acids can be introduced into the wells, e.g., a clonal population of single nucleic can be attached to a single bead, and the bead can be introduced into a well. To initiate sequencing of the nucleic acids on the beads, one type of deoxyribonucleotide (e.g., dATP, dCTP, dGTP, or dTTP) can be introduced into the wells. When one or more nucleotides are incorporated by DNA polymerase, protons (hydrogen ions) are released in the well, which can be detected by the ion sensor. The semiconductor chip can then be washed and the process can be repeated with a different deoxyribonucleotide. A plurality of nucleic acids can be sequenced in the wells of a semiconductor chip. The semiconductor chip can comprise chemical-sensitive field effect transistor (chemFET) arrays to sequence DNA (for example, as described in U.S. Patent Application Publication No. 20090026082). Incorporation of one or more triphosphates into a new nucleic acid strand at the 3′ end of the sequencing primer can be detected by a change in current by a chemFET. An array can have multiple chemFET sensors.

### Genetic Analysis

The methods of the present invention can be used in the analysis of genetic information of selective genomic regions of interest as well as genomic regions which may interact with the selective region of interest. Amplification methods as disclosed herein can be used in the devices, kits, and methods known to the art for genetic analysis, such as, but not limited to those found in U.S. Pat. Nos. 6,449,562, 6,287,766, 7,361,468, 7,414,117, 6,225,109, and 6,110,709. In some cases, amplification methods of the present invention can be used to amplify target nucleic acid of interest for DNA hybridization studies to determine the presence or absence of polymorphisms. The polymorphisms, or alleles, can be associated with diseases or conditions such as genetic disease. In other cases the polymorphisms can be associated with susceptibility to diseases or conditions, for example, polymorphisms associated with addiction, degenerative and age related conditions, cancer, and the like. In other cases, the polymorphisms can be associated with beneficial traits such as increased coronary health, or resistance to diseases such as HIV or malaria, or resistance to degenerative diseases such as osteoporosis, Alzheimer's or dementia.

### Kits

Any of the compositions described herein may be comprised in a kit. In a non-limiting example, the kit, in a suitable container, comprises: an adaptor or several adaptors, one or more of oligonucleotide primers and reagents for ligation, primer extension and amplification. The kit may also comprise means for purification, such as a bead suspension.

The containers of the kits will generally include at least one vial, test tube, flask, bottle, syringe or other containers, into which a component may be placed, and preferably, suitably aliquotted. Where there is more than one component in the kit, the kit also will generally contain a second, third or other additional container into which the additional components may be separately placed. However, various combinations of components may be comprised in a container.

When the components of the kit are provided in one or more liquid solutions, the liquid solution can be an aqueous solution. However, the components of the kit may be provided as dried powder(s). When reagents and/or components are provided as a dry powder, the powder can be reconstituted by the addition of a suitable solvent.

A kit will preferably include instructions for employing, the kit components as well the use of any other reagent not included in the kit. Instructions may include variations that can be implemented.

In one aspect, the invention provides kits containing any one or more of the elements disclosed in the above methods and compositions. In some embodiments, a kit comprises a composition of the invention, in one or more containers. In some embodiments, the invention provides kits comprising adapters, primers, and/or other oligonucleotides described herein. In some embodiments, the kit further comprises one or more of: (a) a DNA ligase, (b) a DNA-dependent DNA polymerase, (c) an RNA-dependent DNA polymerase, (d) a forward adapter (e) one or more oligonucleotides comprising reverse adaptor sequence and (f) one or more buffers suitable for one or more of the elements contained in said kit. The adapters, primers, other oligonucleotides, and reagents can be, without limitation, any of those described above. Elements of the kit can further be provided, without limitation, in any of the amounts and/or combinations (such as in the same kit or same container) described above. The kits may further comprise additional agents, such as those described above, for use according to the methods of the invention. For example, the kit can comprise a first forward adaptor that is a partial duplex adaptor as described herein, a second forward adapter, and a nucleic acid modifying enzyme specific for a restriction and/or cleavage site present in the first forward adaptor. The kit elements can be provided in any suitable container, including but not limited to test tubes, vials, flasks, bottles, ampules, syringes, or the like. The agents can be provided in a form that may be directly used in the methods of the invention, or in a form that requires preparation prior to use, such as in the reconstitution of lyophilized agents. Agents may be provided in aliquots for single-use or as stocks from which multiple uses, such as in a number of reaction, may be obtained.

In one embodiment, the kit comprises a plurality of forward adaptor oligonucleotides, wherein each of said forward adaptor oligonucleotides comprises at least one of a plurality of barcode sequences, wherein each barcode sequence of the plurality of barcode sequences differs from every other barcode sequence in said plurality of barcode sequences at at least three nucleotide positions, and instructions for using the same. Forward adapters comprising different barcode sequences can be supplied individually or in combination with one or more additional forward adapters having a different barcode sequence. In some embodiments, the kit can comprises a plurality of first and second forward adapter oligonucleotides. Second forward adapter oligonucleotides can be supplied separately from or in combination with one or more first forward adapters, and/or one or more different second adapters. Combinations of first and second forward adapters can be supplied in accordance with combinations described above. In some embodiments, the kit can comprises a plurality of oligonucleotides comprising reverse adaptor sequence. In one embodiment, the kit can comprises a plurality of oligonucleotides comprising reverse adaptor sequence, wherein each of the plurality of oligonucleotides comprising reverse adaptor sequence further comprises sequence complementary to a specific target sequence of interest present in a nucleic acid. In one embodiment, the kit can comprises a plurality of oligonucleotides comprising reverse adaptor sequence, wherein each of the plurality of oligonucleotides comprising reverse adaptor sequence further comprises random sequence. In one embodiment, the kit comprises a plurality of oligonucleotides with reverse adaptor sequence, wherein each of said oligonucleotides with reverse adaptor sequence comprises at least one of a plurality of barcode sequences, wherein each barcode sequence of the plurality of barcode sequences differs from every other barcode sequence in said plurality of barcode sequences at at least three nucleotide positions, and instructions for using the same. Oligonucleotides with reverse adaptor sequence comprising different barcode sequences can be supplied individually or in combination with one or more additional oligonucleotides with reverse adaptor sequence having a different barcode sequence.

In some cases, methods, compositions, and/or kits described herein can be used to detect a fusion event (e.g., any fusion event). In some cases, the fusions can be a fusion found in cancer. In some cases, the cancer fusion is a fusion as curated by the Wellcome Trust Sanger Institute's Catalog of Somatic Mutations in Cancer (COSMIC). (//www.sanger.ac.uk/resources/databases/cosmic.html). In some cases, the number of unique fusions that can be detected in a single assay is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500. In some cases, the number of unique cancer fusions that can be detected in a single assay is about 1 to about 10, about 5 to about 25, about 20 to about 50, about 25 to about 50, about 50 to about 100, about 100 to about 200, about 100 to about 300, about 100 to about 400, about 100 to about 450, about 100 to about 500, about 200 to about 300, about 200 to about 400, about 200 to about 500, about 300 to about 400, or about 300 to about 500. In some cases, the number of unique cancer fusions that be detected in a single assay is about 10, 25, 50, 75, 100, 125, 150, 175, 200, 225, 250, 275, 300, 325, 350, 375, 400, 425, 450, 475 or 500. In some cases, fusions are detected using genomic nucleic acid (e.g., genomic DNA) as a starting material. In some cases, fusions are detected using RNA (e.g., messenger RNA) as a starting material. In some cases, the methods and compositions described herein can be used to enrich for and detect gene fusion events in a nucleic acid sample. In these examples, a target specific primer or oligonucleotide may anneal to a portion of at least one gene in the gene fusion. In other examples, a target specific primer or oligonucleotide may anneal to a portion of both genes in the gene fusion (i.e., at the boundary of the gene fusion event).

In some cases, a target specific primer anneals to an exon of a gene. In some cases, a target specific primer anneals to an intron of a gene. In some cases, each of a plurality of target specific primers anneals an exon in a unique gene. In some cases, a primer anneals to a sequence spanning an exon/intron junction. In some cases, a single assay can employ a plurality of primers, wherein the primers anneal to at least 10, 50, 100, 200, 300, 400, 500, 600, 700, 800, 900, 1000, 2000, 3000, 4000, 5000, 6000, 7000, 8000, 9000, 10,000, 20,000, 30,000, 40,000, or 50,000 unique exons. In some cases, a single assay employs a plurality of primers that anneal to about 5769 exons. In some cases, a single assay employs a plurality of primers that anneal to about 100 to about 500, about 500 to about 1000, about 1000 to about 2000, about 2000 to about 3000, about 3000 to about 4000, about 4000 to about 10,000, about 5000 to about 10,000, about 4000 to about 6000, or about 5000 to about 7500 unique exons.

A kit can comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, or 1000 different barcodes. A kit can comprise at least 8, 32, or 96 barcodes. A kit can comprise 8, 32, or 96 barcodes.

In some cases, the methods, compositions, and/or kits described herein can be used to discover novel gene fusions. In some cases, the methods, compositions, and/or kits described herein can be used to detect alternative splice forms. The methods, compositions, and/or kits described herein can be used to detect a low abundance of gene fusion transcripts. The amount of starting material can be, e.g., about, or at least 0.1 ng, 0.5 ng, 1 ng, 5 ng, 10 ng, 20 ng, 30 ng, 40 ng, 50 ng, 75 ng, 100 ng, 200 ng, 300 ng, 400 ng, 500 ng, 750 ng, 1000 ng, 5000 ng, or 10,000 ng of nucleic acid, e.g., RNA. The amount of starting material can be, e.g., about 0.1 ng to about 1 ng, about 1 ng to about 10 ng, about 10 ng, about 10 ng to about 50 ng, about 10 ng to about 100 ng, about 10 ng to about 500 ng, about 10 ng to about 1000 ng, about 100 ng to about 500 ng, or about 100 ng to about 1000 ng. In some cases, the amount of starting material is about or less than 10 ng of total RNA. In some cases, the amount of starting material is about or greater than 10 ng of total RNA. In some cases, nucleic acid (e.g., RNA) is obtained from a fresh sample. In some cases, nucleic acid is obtained from a formalin-fixed tissue. In some cases, the sample is a formalin-fixed paraffin-embedded tissue.

In some cases, the methods, compositions, and/or kits described herein can be used for basic research. In some cases, the methods, compositions, and/or kits described herein can be used for in RNA-Seq based cancer diagnostic and/or prognostic tests.

In some cases, a nucleic acid sample comprises a fusion of genetic material (e.g., a translocation). In some cases a fusion can be in cancer. In some cases, the cancer is acute myeloid leukemia; bladder cancer, including upper tract tumors and urothelial carcinoma of the prostate; bone cancer, including chondrosarcoma, Ewing's sarcoma, and osteosarcoma; breast cancer, including noninvasive, invasive, phyllodes tumor, Paget's disease, and breast cancer during pregnancy; central nervous system cancers, adult low-grade infiltrative supratentorial astrocytoma/oligodendroglioma, adult intracranial ependymoma, anaplastic astrocytoma/anaplastic oligodendroglioma/glioblastoma multiforme, limited (1-3) metastatic lesions, multiple (>3) metastatic lesions, carcinomatous lymphomatous meningitis, nonimmunosuppressed primary CNS lymphoma, and metastatic spine tumors; cervical cancer; chronic myelogenous leukemia (CML); colon cancer, rectal cancer, anal carcinoma; esophageal cancer; gastric (stomach) cancer; head and neck cancers, including ethmoid sinus tumors, maxillary sinus tumors, salivary gland tumors, cancer of the lip, cancer of the oral cavity, cancer of the oropharynx, cancer of the hypopharynx, occult primary, cancer of the glottic larynx, cancer of the supraglottic larynx, cancer of the nasopharynx, and advanced head and neck cancer; hepatobiliary cancers, including hepatocellular carcinoma, gallbladder cancer, intrahepatic cholangiocarcinoma, and extrahepatic cholangiocarcinoma; Hodgkin disease/lymphoma; kidney cancer; melanoma; multiple myeloma, systemic light chain amyloidosis, Waldenstrom's macroglobulinemia; myelodysplastic syndromes; neuroendocrine tumors, including multiple endocrine neoplasia, type 1, multiple endocrine neoplasia, type 2, carcinoid tumors, islet cell tumors, pheochromocytoma, poorly differentiated/small cell/atypical lung carcinoids; Non-Hodgkin's Lymphomas, including chronic lymphocytic leukemia/small lymphocytic lymphoma, follicular lymphoma, marginal zone lymphoma, mantle cell lymphoma, diffuse large B-Cell lymphoma, Burkitt's lymphoma, lymphoblastic lymphoma, AIDS-Related B-Cell lymphoma, peripheral T-Cell lymphoma, and mycosis fungoides/Sezary Syndrome; non-melanoma skin cancers, including basal and squamous cell skin cancers, dermatofibrosarcoma protuberans, Merkel cell carcinoma; non-small cell lung cancer (NSCLC), including thymic malignancies; occult primary; ovarian cancer, including epithelial ovarian cancer, borderline epithelial ovarian cancer (Low Malignant Potential), and less common ovarian histologies; pancreatic adenocarcinoma; prostate cancer; small cell lung cancer and lung neuroendocrine tumors; soft tissue sarcoma, including soft-tissue extremity, retroperitoneal, intra-abdominal sarcoma, and desmoid; testicular cancer; thymic malignancies, including thyroid carcinoma, nodule evaluation, papillary carcinoma, follicular carcinoma, Hiirthle cell neoplasm, medullary carcinoma, and anaplastic carcinoma; uterine neoplasms, including endometrial cancer or uterine sarcoma.

In some cases, the methods described herein can be used to assess the expression level of a gene. In some cases, the expression level can be, for example, higher than normal, normal, or below normal. In some cases, a nucleic acid analyzed using a method, compositions, or kit described herein lack a mutation (e.g., wild-type) or contains one or more mutations (e.g., de novo mutation, nonsense mutation, missense mutation, silent mutation, frameshift mutation, insertion, substitution, point mutation, single nucleotide polymorphism (SNP), deletion, rearrangement, amplification, chromosomal translocation, interstitial deletion, chromosomal inversion, loss of heterozygosity, loss of function, gain of function, dominant negative, or lethal). In some cases, methods, compositions or kits described herein can be used to determine copy number alteration or a copy number variation.

Fusion-Caused Cancers

In some cases, fusion genes can be found in cancer. Cancers in which a gene fusion can be found include acute lymphoblastic leukemia (ALL), acute nonlymphocytic leukemia (ANLL), acute myelogenous, acute myelogenous leukemia (AML), congenital fibrosarcoma (CFS), congenital mesoblastic nephroma (CMN), secretory ductal carcinoma of breast, congenital mesoblastic nephroma (CMN), angiomatoid fibrous histiocytoma (AFH), myxoid liposarcoma (MLS), acute myelomonocytic, alveolar rhabdomyosarcoma (ARMS), acute promyelocytic leukemia (APL), anaplastic large cell lymphoma (ALCL), B-cell non-Hodgkin lymphoma (NHL), and marginal zone B-cell lymphoma (MZBCL) or mucosa-associated lymphoid tissue (MALT).

Cancer Gene Fusions

In some cases, a cancer fusion can be TEL-AML1 fusion, E2A-PBX (PBX1) fusion, BCR-ABL fusion, MLL-AF4 fusion, IGH-MYC fusion, and TCR-RBTN2 fusion in ALL. In other cases, a cancer fusion can be RET and NTRK1 fusion in papillary thyroid carcinoma, PAX8-PPARG in follicular thyroid carcinoma, MECT1-MAML2 in mucoepidermoid carcinoma, TFE3 and TFEB fusion in kidney carcinomas, BRD4-NUT in midline carcinomas, ETV6-NTRK3 in secretory breast carcinomas, TMPRSS2-ETS fusion in prostate carcinomas, and TMPRSS2-ERG in prostate cancer. In certain embodiments, a cancer fusion can be a MLL-AF4 fusion in ALL, MLL-MLLT3 fusion in ANLL, MLL-MLLT1 fusion in ALL and ANLL, MLL-CREBBP fusion in AML, ABL-BCR fusion in CML, ALL, and ANLL, ETV6-NTRK3 fusion in CFS, and CMN, ETV6-RUNX1 fusion in B cell ALL, FKHRL1-PAX3 fusion in ARMS, RARA-PML fusion in APL, RARA-NPM1 fusion in ALCL, or DDX10-NUP98 fusion in MDS and ANLL. In some cases, the cancer fusion is a RET fusion. In other cases, the cancer fusion is a ROS1 fusion. In other cases, the cancer fusion is an ALK fusion.

Non-limiting examples of genes that can be involved in cancer fusions include: ABI1, ABL1, ABL2, ACBD6, ACCN1, ACLY, ACSL3, ACTB, AF15Q14, AF1Q, AF3p21, AFF4, AGPAT5, AGTRAP, AHRR, AKAP9, ALDH2, ALK, ALO17, AMD1, ARFGEF2, ARFIP1, ARHGAP6, ARHGEF12, ARHH, ARID1A, ARNT, ASPSCR1, ASXL1, ATF1, ATG4C, ATIC, ATP8B2, BAIAP2L1, BBS9, BCAP29, BCAS3, BCAS4, BCL10, BCL11A, BCL11B, BCL2, BCL3/5, BCL6, BCL7A, BCL9, BCOR, BCR, BIRC3, BRAF, BRD3, BRD4, BTG1, C15orf21, C16orf75, C2orf44, CACNA2D4, CAMTA1, CANT1, CARS, CBFA2T3, CBFB, CBL, CCDC6, CCNB1IP1, CCND1, CCND2, CCND3, CD273, CD274, CD74, CDH11, CDK6, CDR1, CDX1, CDX2, CEP1, CEP85L, CHCHD7, CHD7, CHEK2, CHIC2, CHN1, CIC, CIITA, CLCN6, CLDN18, CLK2, CLTC, CLTCL1, CMKOR1, COL1A1, COL1A2, COL6A3, COX6C, CPB2, CREB1, CREB3L1, CREB3L2, CREBBP, CRLF2, CRTC1, CRTC3, CSF1, CTAGE5, CTNNB1, CUTA, CXorf67, CYP39A1, DAZL, DDIT3, DDX10, DDX5, DDX6, DEK, DUS4L, EBF1, EFNA5, EHF, EIF3E, EIF3K, EIF4A2, EIF4E2, ELF4, ELK4, ELL, ELN, EML1, EML4, EP300, EPC1, EPS15, ERC1, ERCC2, ERG, ERO1L, ESRP1, ETS1, ETV1, ETV4, ETV5, ETV6, EWSR1, EZR, FACL6, FAM131B, FAM22A, FBXL18, FBXO38, FCGR2B, FCHSD1, FERMT2, FEV, FGFR1, FGFR1OP, FGFR3, FHDC1, FHIT, FIP1L1, FLI1, FLII, FLJ27352, FLT3, FN1, FNBP1, FOXO1A, FOXO3A, FOXP1, FSTL3, FUS, FVT1, GAB2, GABBR2, GASS, GAS6, GAS7, GLI1, GMDS, GMPS, GNAI1, GOLGA5, GOPC, GPBP1L1, GPC3, GPHN, GRAF, GSTP1, HAS2, HCMOGT-1, HEAB, HERPUD1, HEY1, HIP1, HIST1H4I, HJURP, HLF, HLXB9, HMGA1, HMGA2, HN1, HNRNPA2B1, HOOK3, HOXA11, HOXA13, HOXA9, HOXC11, HOXC13, HOXD11, HOXD13, HSPCA, HSPCB, IKZF1, IL2, IL21R, IL6R, INTS4, IQCG, IRF2BP2, IRF4, IRTA1, ITK, ITPR2, JAK2, JAZF1, KDELR2, KDM5A, KIAA0284, KIAA1549, KIF3B, KIF5B, KLC1, KLK2, KRAS, KTN1, LAF4, LASP1, LCK, LCP1, LCX, LGR5, LHFP, LIFR, LMO1, LMO2, LPP, LRIG3, LYL1, MACROD1, MAF, MAFB, MALAT1, MALT1, MAML2, MARK4, MAST1, MAST2, MBOAT2, MBTD1, MCPH1, MDS2, MEAF6, MECOM, MET, MIPOL1, MKL1, MKRN1, MLF1, MLL, MLLT1, MLLT10, MLLT2, MLLT3, MLLT4, MLLT6, MLLT7, MN1, MSF, MSI2, MSN, MTCP1, MUC1, MYB, MYC, MYH11, MYH9, MYO1C, MYO1F, MYST4, NAB2, NACA, NACC2, NCOA1, NCOA2, NCOA4, NDRG1, NF1, NFATC1, NFATC2, NFIA, NFIB, NFIX, NFKB2, NIN, NONO, NOP2, NOTCH1, NPM1, NR4A3, NRG1, NSD1, NTN1, NTRK1, NTRK2, NTRK3, NUMA1, NUP107, NUP214, NUP98, NUTM1, ODZ4, OLIG2, OMD, OMG, OSCP1, P2RY8, PACS1, PAFAH1B2, PAFAH1B3, PAX3, PAX5, PAX7, PAX8, PBX1, PCM1, PCSK7, PDE4DIP, PDE8B, PDGFB, PDGFRA, PDGFRB, PERI, PHF1, PICALM, PIM1, PKD1L1, PLA2R1, PLAG1, PLXND1, PML, PMX1, PNUTL1, POU2AF1, POU5F1, PPARG, PPFIBP1, PPP1R1B, PPP2R2A, PRCC, PRDM16, PRDX4, PRKAR1A, PRKAR2A, PRKCE, PRKG2, PSIP2, PTPRK, PTPRZ1, QKI, RAB5EP, RAD51L1, RAF1, RALGDS, RANBP17, RAP1GDS1, RARA, RASA3, RBM14, RBM15, RBMS1, RET, RGS17, RGS22, RLF, RNF130, RNF216, ROD1, ROS1, RPL22, RPN1, RRP15, RSPO2, RSPO3, RUNDC2A, RUNX1, RUNX1T1, RUNXBP2, SDC4, SEC16A, SEC31A, SEPT6, SET, SFPQ, SFRS3, SH3GL1, SIL, SIP1, SLC22A1, SLC26A11, SLC26A6, SLC34A2, SLC45A3, SLCO1B3, SMARCA5, SNAP91, SP3, SPTBN1, SQSTM1, SRGAP3, SS18, SS18L1, SSBP2, SSH2, SSX1, SSX2, SSX4, STARD3, STAT5B, STATE, STL, STRN, SULF2, SUSD1, SUZ12, SYCP1, SYK, SYT1, TACC1, TACC3, TADA2A, TAF15, TAL1, TAL2, TAS2R38, TBCEL, TBL1XR1, TCEA1, TCF12, TCF3, TCF7L1, TCF7L2, TCL1A, TCL6, TECTA, TFE3, TFEB, TFG, TFPT, TFRC, THRAP3, TLX1, TLX3, TMCC1, TMPRSS2, TNFRSF17, TNFRSF1B, TOP1, TOP2B, TOX4, TPM3, TPM4, TPR, TRIM24, TRIM27, TRIM33, TRIP11, TTL, UBE2L3, USH1G, USP6, VCL, VTI1A, WHSC1, WHSC1L1, WIF1, WT1, WWTR1, XKR3, YWHAE, YY1, ZC3H7B, ZNF145, ZNF198, ZNF278, ZNF331, ZNF384, ZNF444, ZNF521, ZNF700, ZNF703, and ZNF9.

### Products Based on the Methods of the Invention

Products based on the methods of the invention may be commercialized by the Applicants under the trade name Ovation®. Ovation® is a trademark of NuGEN Technologies, Inc.

### EXAMPLES

### Example 1

### Characterization of the Human Oral Microbiome by Selective Enrichment of Bacterial 16S Ribosomal DNA Sequences

Sample Nucleic Acid

Microbial genomic DNA is isolated from human saliva using the OMNIgene-DISCOVER sample collection kit (DNA Genotek) according to the manufacturer's instructions. Extracted DNA is then fragmented via sonication to an average length of 400 bp and purified using Agencourt AMPure XP beads (Beckman Coulter Genomics).

Generation of Control and Test Libraries with Ligated Forward Adaptors

The NuGEN Ovation Ultralow Library System (NuGEN Technologies) is used to generate two next generation sequencing libraries from 100 ng of the purified sample. The first library, an unenriched control, is made as recommended by the manufacturer. A second ‘test’ library, the input for downstream enrichment steps, is generated using the same library construction kit modified as follows. Briefly, DNA is blunted and prepared for ligation under the standard end-repair reaction conditions described in the kit. Fragments are then ligated to the forward adaptor only. As depicted in FIG. 2, ligation attaches the forward adaptor to each end of each DNA fragment, leaving a single-strand nick on the opposite strand. Adaptor fill-in will be performed, thus generating ligation products where complementary ends exist on each insert.

Ligation products of at least 100 bp in length are purified by selective binding to Agencourt AMPure XP beads and taken forward into the enrichment process.

Amplification

Ribosomal DNA fragments from the test library are selectively amplified with two distinct steps: 1) gene-specific primer extension; and 2) PCR with universal adaptor sequences. The primer extension step is performed with oligonucleotides containing a 3′ gene-specific region and a 5′common region that contains a portion of the Illumina reverse adaptor sequence. Consensus 16S sequences making up the gene-specific segment are selected by comparing the ribosomal operons from 40 diverse bacterial species using the ClustalW multiple sequence alignment program (European Bioinformatics Institute). Oligonucleotides representing each of the 18 highly conserved sequence blocks identified across the 16S genomic loci are synthesized and mixed in equimolar proportions.

The pool of primer extension probes is combined with the test DNA library (above) containing the forward adaptor and the HotStarTaq PCR mastermix (QIAGEN, USA) containing buffer, dNTPs, and a thermally-activated Taq DNA polymerase. This solution is placed in a thermal cycler, heated to 95° C. for 15 minutes to activate the polymerase and cooled to 70° C. for 5 minutes to allow the 16S primers to anneal to DNA inserts and extend into the forward adaptor site. Amplification primers that bind to the forward and reverse adaptor sites are added. Selection for fragments that contain both the forward (test library) and reverse (5′ common region on 16S primers) adaptor, and the respective universal priming sites, is accomplished with PCR using a 3-step temperature routine (94° C. for 30 seconds, 60° C. for 30 seconds, 72° C. for 1 minute) for 25 cycles. PCR products are purified using AMPure XP beads and analyzed with a 2100 Bioanalyzer (Agilent Technologies).

Sequencing and Data Analysis

Single end sequencing reads of 100 nt length are obtained for both the control and enriched test libraries using a MiSeq System (Illumina) Raw sequencing data is processed using Illumina base calling software and mapped to a ribosomal RNA database. Sequences that do not align to bacterial rRNA are mapped to human and bacterial full genome reference sequences. Fold enrichment is determined by calculating the number of rRNA reads as a percentage of total mapped reads in the control and test samples.

### Example 2

### Characterization of Changes Over Time to the Human Oral Microbiome by Selective Enrichment of Bacterial 16S Ribosomal DNA Sequences

Sample Nucleic Acid

Microbial genomic DNA is isolated from human saliva using the OMNIgene-DISCOVER sample collection kit (DNA Genotek) according to the manufacturer's instructions at 1 hour intervals for 16 hours following use of dental rinse. Extracted DNA is then fragmented via sonication to an average length of 400 bp and purified using Agencourt AMPure XP beads (Beckman Coulter Genomics).

Generation of DNA Fragments with Ligated Forward Adapters

Components from the NuGEN Ovation Ultralow Library System (NuGEN Technologies) are used to generate 16 independent next generation sequencing libraries from 100 ng of the purified sample. Briefly, DNA is blunted and prepared for ligation under the standard end-repair reaction conditions described in the kit. Fragments are then ligated to the forward adapter only. As depicted in FIG. 2, ligation attaches the forward adapter to each end of each DNA fragment, leaving a single-strand nick on the opposite strand. Adapter fill-in will be performed, thus generating ligation products where complementary ends exist on each insert.

Ligation products of at least 100 bp in length are purified by selective binding to Agencourt AMPure XP beads and taken forward into the enrichment process.

Primer Extension

Libraries containing ribosomal genes are generated by introducing the reverse adapter attached to the 5′ end of oligonucleotides specific to conserved regions within these genes. There are two distinct steps: 1) annealing of the gene-specific primer; and 2) extension of that primer through the action of a DNA polymerase. The resulting product is a functional library containing the forward adapter on one end and the reverse adapter on the other end. The gene-specific primer extension step is performed with oligonucleotides containing a 3′ gene-specific region and a 5′ region that contains a portion of the Illumina reverse adapter sequence. Embedded in the reverse adapter sequence is a variable region of 8 bases that differentiates this adapter from the 16 other adapters used with the other samples. Thus, 16 gene-specific libraries have been generated; one from each sample. Each library has a common forward adapter. Each library also contains a common sequence on the opposite end but within that common sequence there is a unique 8 nucleotide region. Consensus 16S sequences making up the gene-specific segment are selected by comparing the ribosomal operons from 40 diverse bacterial species using the ClustalW multiple sequence alignment program (European Bioinformatics Institute). Oligonucleotides representing each of the 18 highly conserved sequence blocks identified across the 16S genomic loci are synthesized and mixed in equimolar proportions.

Individual samples with forward adapters ligated onto each strand are combined with the primer extension probes (described above) in 16 independent reactions. These are mixed with HotStarTaq PCR mastermix (QIAGEN, USA) containing buffer, dNTPs, and a thermally-activatable Taq DNA polymerase. This solution is placed in a thermal cycler, heated to 95° C. for 15 minutes to activate the polymerase and cooled to 70° C. for 5 minutes to allow the 16S primers to anneal to DNA inserts and extend into the forward adapter site.

Amplification

The 16 individual primer extension products (above) are pooled, amplification primers that are complementary to the 5′ ends of the forward and reverse adapter sites but also contain portions complementary to flow cell oligonucleotide sequences are added. Selection for fragments that contain both the forward and reverse (5′ common region on 16S primers) adapter, and the respective universal priming sites, is accomplished with PCR using a 3-step temperature routine (94° C. for 30 seconds, 60° C. for 30 seconds, 72° C. for 1 minute) for 25 cycles. PCR products are purified using AMPure XP beads and analyzed with a 2100 Bioanalyzer (Agilent Technologies).

Sequencing and Data Analysis

Single end sequencing reads of 100 nt length are obtained for both the control and enriched test libraries using a MiSeq System (Illumina) Raw sequencing data is processed using Illumina base calling software. Samples from the various time points are binned based on their unique 8 base code and mapped to a ribosomal RNA database. Sequences that do not align to bacterial rRNA are mapped to human and bacterial full genome reference sequences. Changes in microbial populations are assessed by comparing 16S read counts from the different organisms in the samples over time.

### Example 3

### Characterization of Transcriptional Activity of Individual Cells within a Population

Sample Nucleic Acid

Individual cells are isolated from whole blood using a FACS cell sorter. The cells are suspended in 10 μl of Prelude Lysis solution (a component of NuGEN Technologies, One Direct system), resulting in lysis of the cell membrane while the nuclear membrane remains intact. Sixteen of the single cell suspensions are selected for expression profiling. Briefly, kit reagents are used as described by the manufacturer to generate first and second strand cDNA from the total RNA present in the lysate. Double stranded cDNA products are purified using Agencourt AMPure XP beads (Beckman Coulter Genomics).

Generation of Fragments with Ligated Forward Adapters

Components from the NuGEN Ovation Ultralow Library System (NuGEN Technologies) are used to generate next generation sequencing libraries from each of the purified sample. Briefly, DNA is blunted and prepared for ligation under the standard end-repair reaction conditions described in the kit. Fragments are then ligated to the forward adapter only. As depicted in FIG. 2, ligation attaches the forward adapter to each end of each DNA fragment, leaving a single-strand nick on the opposite strand. Adapter fill-in will be performed, thus generating ligation products where complementary ends exist on each insert.

Ligation products of at least 100 bp in length are purified by selective binding to Agencourt AMPure XP beads and taken forward into library generation.

Primer Extension

Libraries are generated by introducing the reverse adapter attached to the 5′ end of a random hexamer. There are two distinct steps: 1) annealing of the primer; and 2) extension of that primer through the action of a DNA polymerase. The resulting product is a functional library containing the forward adapter on one end and reverse adapter on the other end. The primer extension step is performed with oligonucleotides containing a 3′ random region and a 5′ region that contains a portion of the Illumina reverse adapter sequence. Embedded in the reverse adapter sequence is a variable region of 8 bases that differentiates this adapter from the 16 other adapters used with the other samples. Thus, 16 libraries have been generated; one from each sample. Each library has a common forward adapter. Each library also contains a common sequence on the opposite end but within that common sequence there is a unique 8 nucleotide region.

Individual samples with forward adapters ligated onto each strand are combined with the primer extension probes (described above) in 16 independent reactions. These are mixed with HotStarTaq PCR mastermix (QIAGEN, USA) containing buffer, dNTPs, and a thermally-activatable Taq DNA polymerase. This solution is placed in a thermal cycler, heated to 95° C. for 15 minutes to activate the polymerase and cooled to 70° C. for 5 minutes to allow the primers to anneal to DNA inserts and extend into the forward adapter site.

Amplification

Amplification primers that are complementary to the 5′ ends of the forward and reverse adapter sites but also contain portions complementary to flow cell oligonucleotide sequences are added to the 16 individual primer extension products (above). Selection for fragments that contain both the forward and reverse adapter, and the respective universal priming sites, is accomplished with PCR using a 3-step temperature routine (94° C. for 30 seconds, 60° C. for 30 seconds, 72° C. for 1 minute) for 25 cycles. PCR products are purified using AMPure XP beads and analyzed with a 2100 Bioanalyzer (Agilent Technologies).

Sequencing and Data Analysis

Equal masses of each of the amplified libraries (above) are pooled and diluted to working concentrations according to manufacturer's recommendations. Single end sequencing reads of 100 nt length are obtained for libraries using a MiSeq System (Illumina) Raw sequencing data is processed using Illumina base calling software. Samples from the various time points are binned based on their unique 8 base code and mapped to a reference database. Based on the mapping characteristics, individual samples or a new pool of samples can be rerun on the sequencer to obtain greater read depth. Samples with poor gene coverage will be eliminated from the pool.

### Example 4

### cDNA Enrichment from Target RNA

First Strand cDNA Synthesis

In some cases the following protocol can be used: First Strand Primer Mix (A1), First Strand Buffer Mix (A2), First Strand Enhancer (A4), First Strand Enzyme Mix (A3) and the Nuclease-free Water (D1) from −20° C. storage. Also remove the Agencourt RNAClean XP Beads from 4° C. storage and place at room temperature. Spin down the contents of A3 and place on ice. Thaw the other reagents at room temperature, mix by vortexing, spin and place on ice. Leave the nuclease-free water at room temperature. Add 2 μL of A1 to a 0.2 mL PCR tube. Add 10-200 ng of total RNA to the primer, bringing the total volume to 6 μL with nuclease-free water if necessary. Mix by pipetting 5 times, spin and place on ice. Place the tubes in a pre-warmed thermal cycler programmed to run Program 1: 65° C. for 1 min.

Immediately remove the tubes from the thermal cycler and snap chill on ice. Prepare First Strand master mix by combining and mixing according to the volumes shown below (volumes listed are for a single reaction):

| First Strand | First Strand | First Strand |
|---|---|---|
| Buffer Mix (A2) | Enhancer Mix (A4) | Enzyme Mix (A3) |
| 2.5 μl | 1 μl | 0.5 μl |

Add 4 μL of the First Strand Master Mix to each sample tube. Mix by pipetting, cap and spin the tubes and place on ice. Place the tubes in a pre-warmed thermal cycler programmed to run Program 2: 25° C. for 5 min, 40° C. for 5 min, 70° C. for 15 min, and hold at 4° C. Remove the tubes from the thermal cycler, spin to collect condensation and place on ice. Continue immediately to the Second Strand cDNA Synthesis.

Second Strand cDNA Synthesis

In some cases, the following protocol can be used: Remove Second Strand Buffer (B1), Second Strand Enzyme Mix (B2), and Second Strand Stop Mix (B3) from −20° C. storage. Spin down the contents of B2 and place on ice. Thaw reagents B1 and B3 at room temperature, mix by vortexing, spin and place on ice. Prepare a master mix by combining B1 and B2 with water (D1) in a 0.5 mL capped tube, according to the volumes shown below (volumes listed are for a single reaction):

| Water | Second Strand | Second Strand |
|---|---|---|
| (D1) | Buffer Mix (B1) | Enzyme Mix (B2) |
| 55.5 μl | 7.5 μl | 2.0 μl |

Add 65 μL of the Second Strand Master Mix to each First Strand reaction tube. Mix by pipetting, spin and place on ice. Place the tubes in a pre-cooled thermal cycler programmed to run Program 3: 16° C. for 60 min, and hold at 4° C. Remove the tubes from the thermal cycler and spin to collect condensation. Add 2 μL of Second Strand Stop Buffer (B3). Mix by pipetting and spin. Continue with cDNA purification.

cDNA Purification

In some cases, the following protocol can be used: Ensure RNAClean XP beads are fully resuspended and at room temperature. Aliquot 120 μl of RNAClean XP beads into a fresh tube for each sample to be purified. In some cases, the beads can be added first to the tube, before the sample is added. Add 78 μl of second strand reaction to the beads and mix by pipetting the full volume at least 7 times. Incubate for 5 minutes at room temperature. Place tubes on a magnet for 5 minutes to collect the beads. Remove and discard 185 μl of supernatant; attempt to avoid discarding any beads. Wash beads by gently pipetting 200 μl of fresh 85% ethanol into each tube while still on the magnet. Allow to sit for 30 seconds; in some cases, allow to sit for no more than 1 minute. Remove all ethanol and allow beads to dry on magnet for 10 minutes. Remove tubes from magnet and fully resuspend beads in 11 μl of DNA Resuspension Buffer. Incubate at room temperature for 2 minutes. Place tubes on magnet for 5 minutes to collect beads. Transfer purified cDNA supernatant into a fresh tube. Proceed to End Repair Step of Ovation Target Enrichment protocol using 10 μl of cDNA as input. In some case, Covaris fragmentation of cDNA can be performed. In other cases, Covaris fragmentation of cDNA can be omitted.

### Example 5

### cDNA Enrichment from Target RNA Using, e.g., Ovation Target Enrichment System

8-, 32-, and 96-Sample Cancer Panel and FFPE Cancer Panel, 32- and 96-Sample Custom Kits

I—Introduction

Methods, compositions and kits provided herein, e.g., the Ovation Target Enrichment System can provide custom target enrichment and multiplex library preparation solution for targeted resequencing. A fully customizable probe design can enable the interrogation in some cases of up to 10 Mb of custom content defined by a user. In some cases, the system can work with as little as 10 ng of fresh/frozen tissue gDNA as well as DNA isolated from FFPE tissue. A single-tube workflow can be completed in 24 hours with an optional fast protocol that can be completed in as little as 8 hours from sample to target enriched library. A barcoding design can allow both a high degree of sample multiplexing and the unambiguous identification of true PCR duplicates. In some cases, paired-end sequencing is not used. Each kit includes reagents for multiplexed target enrichment and Illumina sequencing library creation for 8, 32 or 96 samples, respectively.

B. Workflow

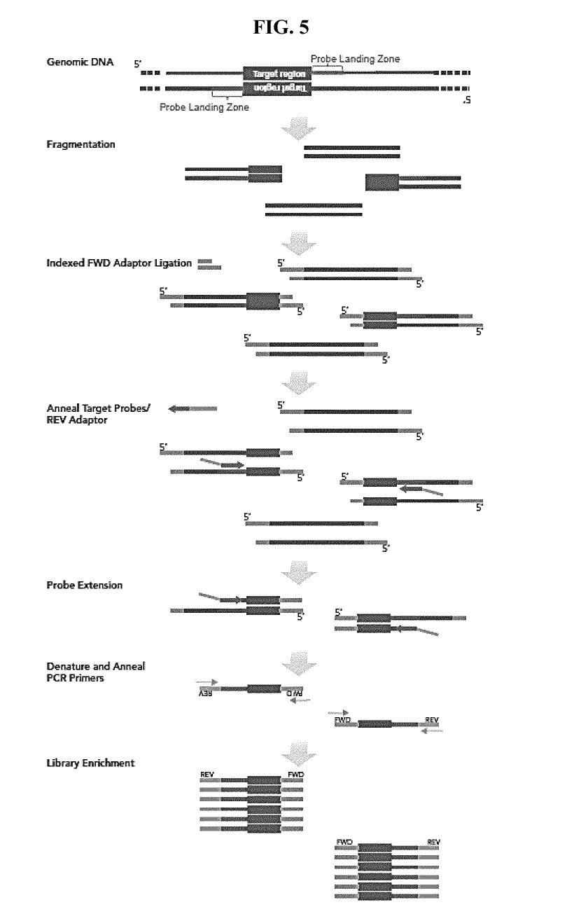

In the workflow shown in FIG. 5, fragmented samples are end-repaired and individually ligated with barcoded adaptors. Following a bead purification step, samples are combined for multiplex target enrichment during which time the targeting probes are annealed and extended, creating amplifiable library molecules. Following a second bead purification, an optional qPCR step can determine the optimal number of PCR cycles to use in the final library amplification step. After library amplification, final bead purification prepares the multiplexed, target enriched library for sequencing. Using this workflow, a single user can easily process 32 samples or more at a time.

C. Performance Specifications

Methods, compositions and kits described herein, e.g., the Ovation Target Enrichment System can produce custom target-enriched, multiplex libraries suitable for sequencing on the Illumina Genome Analyzer IN/He (GAII), MiSeq, NextSeq, HiScan SQ or HiSeq 2000/2500 NGS platforms. The methods, compositions and kits can produce target-enriched libraries from about 10 to about 500 ng fresh or FFPE-derived genomic DNA. Sequencing metrics such as on-target rates and uniformity of coverage can vary from one custom probe design to another.

D. Quality Control

Reagents provided herein, e.g., the Ovation Target Enrichment System can undergo functional testing to assess performance specifications.

E. Storage and Stability

Compositions and kits provided herein, e.g., the Ovation Target Enrichment System can be shipped on dry ice and can be unpacked immediately upon receipt. A product can contain components with multiple storage temperatures. Vials labeled Agencourt® RNAClean® XP Beads (clear cap) can be removed from the top of the shipping carton upon delivery and be stored at 4° C. All other components can be stored at −20° C. on internal shelves of a freezer without a defrost cycle. In some cases, a kit is tested to perform to specifications after as many as six freeze/thaw cycles. Kits handled and stored according to the above guidelines can perform to specifications for at least six months.

II. Components

A. Reagents Provided

TABLE 1
Ovation Target Enrichment Core Library Reagents

| | 0400-08 | 0400-32 | 0400-96 | | |
|---|---|---|---|---|---|
| | PART | PART | PART | VIAL | VIAL |
| COMPONENTS | NUMBER | NUMBER | NUMBER | CAP | NUMBER |
| End Repair Buffer Mix | S01860 | S01899 | S01907 | Blue | ER1 VER 9 |
| End Repair Enzyme Mix | S01510 | S01533 | S01909 | Blue | ER2 VER 4 |
| End Repair Enhancer | S01862 | S01709 | S01908 | Blue | ER3 VER 2 |
| End Repair FFPE Enhancer | S01938 | S01935 | S01939 | Blue | ER5 |
| Ligation Buffer Mix | S01936 | S01534 | S01910 | Yellow | L1 VER 4 |
| Ligation Enzyme Mix | S01467 | S01535 | S01911 | Yellow | L3 VER 4 |
| DNA Resuspension Buffer Mix | S01901 | S01901 (2) | S01901 (3) | Green | DR1 |
| Target Extension Buffer Mix | S01866 | S01900 | S01912 | Clear | TX1 |
| Target Extension Enzyme Mix | S01870 | S01902 | S01913 | Clear | TX2 |
| Amplification Buffer Mix | S01871 | S01903 | S01914 | Red | P1 VER 4 |
| Amplification Primer Mix | S01934 | S01906 | S01917 | Red | P2 VER 14 |
| Amplification Enzyme Mix | S01873 | S01764 | S01895 | Red | P3 VER 2 |
| Amplification Enhancer Mix | S01872 | S01904 | S01915 | Red | P6 |

Ovation Cancer Panel Target Enrichment System Reagents (Part Nos. 0400-08, 0400-32, and 0400-96),

| | | | | | |
|---|---|---|---|---|---|
| Nuclease Free Water | S01001 | S01001, | S01001 (2), | Green | D1 |
| | | S01113 | S01113 (3) | | |
| RNACleanXP Beads | S01502 | S01502 | S01502 | Clear | — |
| Ligation Adaptor Mix | S01941 | N/A | N/A | Yellow | L2V15DR-BC1 |
| | S01942 | | | | L2V15DR-BC2 |
| | S01943 | | | | L2V15DR-BC3 |
| | S01944 | | | | L2V15DR-BC4 |
| | S01945 | | | | L2V15DR-BC5 |
| | S01946 | | | | L2V15DR-BC6 |
| | S01947 | | | | L2V15DR-BC7 |
| | S01948 | | | | L2V15DR-BC8 |
| Target Enrichment | N/A | S01937 | N/A | N/A | N/A |
| 32-Plex Adaptor Plate | | | | | |
| Target Enrichment | N/A | N/A | S01940 | N/A | N/A |
| 96-Plex Adaptor Plate | | | | | |

TABLE 2
Ovation Target Enrichment Probes

| TARGET | 08 RXN | 32 RXN | 96 RXN | | |
|---|---|---|---|---|---|
| ENRICHMENT | KIT PROBE | KIT PROBE | KIT PROBE | VIAL | VIAL |
| SYSTEM | PART NUMBER | PART NUMBER | PART NUMBER | CAP | NUMBER |
| Ovation Cancer | S02007 | S02008 | S02008 (3) | Clear | N/A |
| Panel Target | | | | | |
| Enrichment System | | | | | |
| (Part Nos. 9079-08, | | | | | |
| 9079-32, 9079-96) | | | | | |
| Ovation Cancer | S02009 | S02010 | S02010 (3) | Clear | N/A |
| Panel FFPE Target | | | | | |
| Enrichment System | | | | | |
| (Part Nos. 9099-08, | | | | | |
| 9099-32, 9099-96) | | | | | |
| Ovation Custom | N/A | S02011 | S02011 (3) | Clear | N/A |
| Target Enrichment | | | | | |
| System (Part Nos. | | | | | |
| 9###-32, 9###-96) | | | | | |

B. Additional Equipment, Reagents and Labware

The equipment used in the method in this example can include Covaris Sonication System, Agilent 2100 Bioanalyzer or materials and equipment for electrophoretic analysis of nucleic acids, Real-time PCR system capable of SYBR Green detection, microcentrifuge for individual 1.5 mL and 0.5 mL tubes, microcentrifuge for individual or strip 0.2 mL tubes, vortexer, thermal cycler with 0.2 mL tube heat block, heated lid, and 100 μL reaction capacity, and appropriate spectrophotometer and cuvettes or Nanodrop UV-Vis Spectrophotometer for quantitation of fragmented DNA.

The reagents used in the method in this example can include ethanol (e.g., Sigma-Aldrich, Cat. #E7023) for e.g., purification steps, Eva Green Dye (e.g., Biotium, Cat. #31000) or SYBR Green for, e.g., qPCR

The supplies and labware used in the method in this example can include Amicon Ultra 30K MWCO (e.g., Millipore, Cat. #UFC503024) or vacuum, concentrator, nuclease-free pipette tips, 1.5 mL and 0.5 mL RNase-free microcentrifuge tubes, 0.2 mL individual thin-wall PCR tubes or 8×0.2 mL strip PCR tubes or 0.2 mL, thin-wall PCR plates, magnetic bead separation device, disposable gloves, Kimwipes, ice buckets, cleaning solutions such as DNA-OFF™ (e.g., MP Biomedicals, Cat. #QD0500).

The equipment, reagents, supplies and/or labware can be ordered from companies including Agilent, Inc., Biotium, Covaris, Millipore, MP Biomedicals, and Sigma-Aldrich, Inc.

III. Planning the Experiment

A. Input DNA Requirements

Methods, compositions and kits provided herein, e.g., the Ovation Target Enrichment System can work with inputs of 10 ng to 500 ng of fragmented genomic DNA. DNA samples can be free of contaminating proteins, RNA, organic solvents (e.g., phenol and ethanol) and salts. In some cases, use of a commercially available system for genomic DNA isolation is recommended. In some cases, the A260:A280 ratio for DNA samples should be in excess of 1.8. Use of DNA samples with lower ratios can result in low amplification yield. Genomic DNA derived from FFPE samples can work well in the Ovation Target Enrichment System.

B. Working with the Target Enrichment 32-Plex and 96-Plex Adaptor Plates

Adaptor plates, e.g., the Target Enrichment Adaptor Plates, can contain ligation adaptor mixes, each with a unique eight-base barcode. Each well (first 32 wells (A01-H04) or all 96 wells, respectively) can contain sufficient volume for preparation of a single library. The Target Enrichment Adaptor Plates can be sealed with a foil seal designed to provide for airtight storage. Prior to thawing the adaptor plate, it can be spun for 5 to 10 minutes at room temperature at about 1000×g in a centrifuge with a rotor appropriate for microwell plates. This can allow the plate to thaw while spinning and help to minimize cross-contamination between wells. To use only a portion of the plate, the seal covering only the portion of the plate you wish to use can be removed. The remaining wells of the plate can remain sealed for use at a later date. Used wells can be covered with a new foil seal (e.g., AlumaSeal II) to prevent any remaining adaptor-containing liquid from contaminating future reactions.

C. Enriched, Amplified Library Storage Amplified libraries may be stored at −20° C.

IV. Overview

A. Overview

The library preparation process used in the methods described herein, e.g., the Ovation Target Enrichment System can be performed in four stages: 1. DNA end repair for 0.75 hours; 2. adaptor ligation and purification for 1.75 hours; 3. probe hybridization and extension for from 3 to 12 or more hours (optionally, this step can be performed for about 3 hours); 4. amplification and purification for 2.25 hours. Total time to prepare enriched library can be about 8 to about 20 hours.

Components in kits provided herein, e.g., the Ovation Target Enrichment System, can be color coded, with each color linked to a specific stage of the process. Performing each stage can require making a master mix then adding it to a reaction, followed by incubation. Master mixes can be prepared by mixing components provided for that stage.

B. Protocol Notes

A positive control DNA can be routinely used to establish of a baseline of performance and provide the opportunity for an experimenter to become familiar with the protocol steps.

Water used in this method can be the water provided with a kit (e.g., D1) or an alternate source of nuclease-free water. In some cases, use of DEPC-treated water is not preferred with this protocol.

Components used in each step can be thawed and immediately placed on ice. In some cases, all reagents are not thawed at once.

Thawed reagents and reaction tubes can be kept on ice.

After thawing and mixing buffer mixes, if any precipitate is observed, the mixes can be re-dissolved completely prior to use. The buffer mixes can be gently warmed for 2 minutes at room temperature followed by brief vortexing. In some cases, enzyme mixes are not warmed.

When placing small amounts of reagents into the reaction mix, pipetting up and down several times can be helpful for transfer.

Pipetting a mix can be done by gently aspirating and dispensing a volume that is at least half of the total volume of the reaction mix.

Tubes or plates can be placed in the block after the thermal cycler to reach the initial incubation temperature.

Master mixes can be prepared by using the minimal amount of extra material to help run the maximal number of reactions using the components provided in a kit.

In some cases, the Ovation Target Enrichment Systems are not used with components and reagents from other kits, e.g., kits produced by NuGEN.

The ethanol mixes can be made fresh by measuring both the ethanol and water with pipettes. In some cases, fresh ethanol stocks are used to make 85% ethanol used in the purification steps.

C. Agencourt® RNAClean® XP Purification Beads

There can be significant modifications to the Agencourt RNAClean XP beads standard procedure; in some cases, it is preferred to follow the protocols outlined in this user guide for the use of these beads. However, the Beckman Coulter user guide may be reviewed to become familiar with the manufacturer's recommendations.

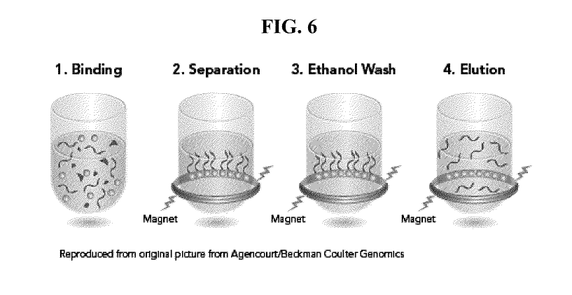

The bead purification processes used in this kit can consist of the following steps (FIG. 6): 1. Binding of DNA to RNAClean XP beads; 2. Magnetic separation of beads from supernatant; 3. Ethanol wash of bound beads to remove contaminants; 4. Elution of bound DNA from beads.

Tips and Notes

Beads can be removed from 4° C. and be left at room temperature for at least 30 minutes before use, and in some case, the reagents can have completely reached room temperature. Cold beads can reduce recovery. Beads can fully resuspended by inverting and tapping before adding to sample. The beads can be separated on the magnet for a full 5 minutes. Binding buffer can be removed before the beads have completely separated will impact DNA yields. After completing the binding step, bead loss can be minimized when removing the binding buffer. Significant loss of beads during the ethanol washes can impact DNA yields, so in some cases it is preferred to minimize bead loss throughout the procedure. The ethanol wash can be freshly prepared from fresh ethanol stocks at the indicated concentration. Lower percent ethanol mixes can reduce recovery. During the ethanol washes, keep the samples on the magnet. In some cases, the beads should not be allowed to disperse; the magnet can keep the beads on the walls of sample wells or tubes in a small ring. All residual ethanol can be removed prior to continuing with the next step. For example, when removing the final ethanol wash, most of the ethanol can first be removed, then the excess can be collected at the bottom of the tube before removing the remaining ethanol. This can reduce the required bead air drying time. After drying the beads for the time specified in the protocol, each tube can be inspected carefully so that the ethanol has evaporated before proceeding. Strip tubes or partial plates can be firmly placed when used with the magnetic plate. In some cases individual tubes are not used because they are difficult to position stably on the magnetic plates.

D. Programming the Thermal Cycler

Use a thermal cycler with a heat block designed for PCR plates, equipped with a heated lid, and with a capacity of 100 μL reaction volume. Prepare the programs shown in Table 3 following the operating instructions provided by the manufacturer. For thermal cyclers with an adjustable heated lid, in some cases the lid temperature can be set to 100° C. only when sample temperature reaches above 30° C. For thermal cyclers with a fixed temperature heated lid (e.g., ABI GeneAmp® PCR 9600 and 9700 models), the default settings (e.g., 100 to 105° C.) can be used.

TABLE 3
Thermal Cycler Programming
END REPAIR

| | |
|---|---|
| Program 1 | 25° C. - 30 min, 70° C. - 10 min, hold at 4° C. |
| End Repair/Ligation | |

DNA REPAIR (OPTIONAL)

| | |
|---|---|
| Program 2 | 16° C. - 10 min, 70° C. - 10 min, hold at 4° C. |
| FFPE DNA Repair | |
| (optional) | |

HYBRIDIZATION

| | |
|---|---|
| Program 3 | 95° C. - 5 min, 200 cycles (80° C. - 10 sec, |
| Probe Hybridization | decrease 0.1° C. each cycle), hold at |
| & Extension | 60° C., 72° C. - 10 min, hold at 4° C. |

AMPLIFICATION

| | |
|---|---|
| Program 4 | 37° C. - 10 min, 95° C. - 3 min, 15-30 cycles* |
| Library Amplification | (95° C. - 30 sec, 62° C. - 15 sec, 72° C. - 20 sec), |
| | 72° C. - 2 min, hold at 4° C. |

*The number of cycles may be adjusted and optimized for desired yield based on qPCR assessment of the unamplified library.

V. Protocol

A. Prepare gDNA

Genomic DNA used in the target enrichment protocol can be fragmented to an average of 500 bp in size. Covaris acoustic shearing can be used following the manufacturer's protocols. The gDNA can be concentrated after fragmentation as the protocol requires the gDNA to be in a volume of 10 μL. The volume of the gDNA can be reduced by using a vacuum concentrator (Speedvac) or an Amicon concentration column according to the manufacturer's instructions. Alternatively, Agencourt AMPure XP or RNAClean XP beads can be used to purify and concentrate the fragmented DNA. Since loss of material can occur during bead purification, quantitation of the samples prior to End Repair can be repeated.

The 8-sample Cancer Panel and FFPE Cancer Panel kits can be used for multiplex enrichment of 4 samples at a time. Processing fewer than 4 samples can result in insufficient Targeting Probe Pool and PCR reagents to complete the reactions. The 32-sample and 96-sample enrichment kits can be used for multiplex enrichment of 8 samples at a time. Processing fewer than 8 samples can result in insufficient Targeting Probe Pool and PCR reagents to complete the reactions. A heated lid can be used to prevent evaporation. PCR tubes or plates that seal tightly and may not deform when the thermal cycler lid is closed can be used. Insufficient tube sealing can result in sample loss due to evaporation during extended incubation steps.

Samples intended to be multiplexed together at Target Enrichment (e.g., step F) can be of equivalent mass, quality and average fragment length. Failure to normalize samples that are processed together can result in unequal sample representation in the final target-enriched library.

B. End Repair

The following end repair process can be used in some cases: 1. The End Repair Buffer Mix (blue: ER1), End Repair Enzyme Mix (blue: ER2) and End Repair Enhancer (blue: ER3) can be obtained from −20° C. storage. 2. ER1 can be thawed at room temperature. Mix by vortexing, spin and place on ice. 3. Spin down contents of ER2 and ER3 and place on ice. 4. Prepare a master mix by combining ER1, ER2 and ER3 in a 0.5 mL capped tube, according to the volumes shown in Table 4. 5. Add 5 μL of the End Repair Master Mix to 10 μL of fragmented DNA (10 ng to 500 ng). 6. Mix by pipetting, cap and spin tubes and place on ice. 7. Place the tubes in a pre-warmed thermal cycler programmed to run Program 1 (End Repair/Ligation; see Table 3): 25° C. −30 min, 70° C. −10 min, and hold at 4° C. 8. Remove the tubes from the thermal cycler, spin to collect condensation and place on ice. 9. Proceed to DNA Repair (optional, Section C) or continue immediately with the Adaptor Ligation protocol (Section D).

TABLE 4
End Repair Master Mix (Volumes listed are for a single reaction)

| END REPAIR | END REPAIR | END REPAIR |
|---|---|---|
| BUFFER MIX | ENZYME MIX | ENHANCER |
| (BLUE: ER1 VER 9) | (BLUE: ER2 VER 4) | (BLUE: ER3 VER 2) |
| 4 μL | 0.5 μL | 0.5 μL |

C. FFPE DNA Repair (Optional)

This is an optional repair step for use with damaged DNA, e.g., FFPE DNA. 1. Add 1 μL of End Repair FFPE Enhancer (ER5). 2. Mix well by pipetting. 3. Place the tubes in a pre-warmed thermal cycler programmed to run Program 2 (FFPE DNA Repair; see Table 3): 16° C. −10 min, 70° C. −10 min, and hold at 4° C. 4. Remove the tubes from the thermal cycler, spin briefly and place on ice.

D. Adaptor Ligation

In some cases, the following adaptor ligation protocol can be used: 1. Remove the Ligation Buffer Mix (yellow: L1), Ligation Enzyme Mix (yellow: L3) and Nuclease-free Water (D1) from −20° C. storage. For 8-reaction kits: Remove Ligation Adaptor Mixes (L2V15DR-BC1 to BC8) from −20° C. storage. For 32- and 96-reaction kits: Remove Target Enrichment Adaptor Plate (32-plex or 96-plex) from −20° C. storage. 2. Thaw L1, Nuclease-free Water and Ligation Adaptor Mixes at room temperature. Mix L1 by vortexing, spin and place on ice. 3. For 8-reaction kits: Spin down Ligation Adaptor Mixes and place on ice. For 32- and 96-reaction kits: Spin down Target Enrichment Adaptor Plate and place on ice. 4. Spin down L3 and place on ice. 5. Add 3 μL of Ligation Adaptor Mix directly to each end-repaired sample. Each tube of Ligation Adaptor Mix and each well of Target Enrichment Adaptor Plates contains adaptors with a unique barcode. 6. Make a master mix by combining the Nuclease-free Water (D1), L1 and L3 in a 0.5 mL capped tube, according to the volumes shown in Table 5. Prepare the master mix by combining water and Ligation Buffer Mix, vortexing to mix, then adding Ligation Enzyme Mix. Mix thoroughly by repeatedly pipetting up and down using a large volume (100-200 μL) pipettor—the larger bore tips used with such pipettors can be used to sufficiently mix the Ligation Master Mix and sample. The L1 Ligation Buffer Mix can be viscous so it can be pipetted slowly. 7. Add 12 μL Ligation Master Mix to each tube of sample/adaptor. Mix thoroughly by repeatedly pipetting up and down using a large volume (100-200 μL) pipettor—the larger bore tips used with such pipettors can be used to sufficiently mix the Ligation Master Mix and sample. 8. Place the tubes in a pre-warmed thermal cycler programmed to run Program 1 (End Repair/Ligation; see Table 3): 25° C. −30 min, 70° C. −10 min, and hold at 4° C. 9. Remove the tubes from the thermal cycler, spin to collect condensation and place on ice. 10. Continue immediately with the Ligation Purification protocol.

TABLE 5
Ligation Master Mix (volumes listed are for a single reaction)

| | LIGATION | LIGATION |
|---|---|---|
| WATER | BUFFER MIX | ENZYME MIX |
| (GREEN: D1) | (YELLOW: L1 VER 4) | (YELLOW: L3 VER 4) |
| 4.5 μL | 6.0 μL | 1.5 μL |

E. Ligation Purification

In some cases, the following ligation purification scheme can be used: Retrieve the Agencourt RNAClean XP Beads from 4° C. and ensure they are at room temperature. Also remove the DNA Resuspension Buffer (DR1) from −20° C. and thaw at room temperature. Resuspend the beads by inverting and tapping the tube. Ensure the beads can be fully resuspended before adding to the sample. After resuspending, the beads do not need to be spun. (An excess of beads can be provided; therefore, it is not necessary to recover any trapped in the cap.) Add 70 μL of room-temperature Nuclease-free Water (D1) to each ligation reaction. Add 80 μL (0.8 volumes) room temperature bead suspension to each sample. Mix thoroughly by pipetting 10 times. Incubate at room temperature for 5 minutes. Transfer the tubes to the magnet plate and let stand 5 minutes to completely clear the solution of beads. Remove 160 μL of the binding buffer and discard it. Leaving some of the volume behind can minimize bead loss at this step. Care may be taken to prevent beads from being removed with the binding buffer or the wash. With the tubes still on the magnet, add 180 μL of freshly prepared 85% ethanol and allow to stand for at least 30 seconds but, in some cases, no more than 1 minute. Remove the ethanol wash using a pipette. Air dry the beads on the magnet for 10 minutes. Inspect each tube to check that all the ethanol has evaporated. All residual ethanol can be removed prior to continuing. Remove the tubes from the magnet. Add 21 μL of DNA Resuspension buffer (DR1) to the dried beads. Mix thoroughly by pipetting to ensure all the beads are resuspended. Incubate for 2 minutes at room temperature. Transfer the tubes to the magnet and let stand for 3 minutes to completely clear the solution of beads. Carefully remove 20 μL of the eluate, ensuring as few beads as possible are carried over. Combine the eluted DNA of 8 samples (combine 4 samples when using the 0400-08 core kit) into a single fresh 1.5 mL tube. Concentrate the DNA by either using an Amicon 0.5 mL 30 k MWCO spin column (e g, Millipore UFC503024) following the manufacturers' recommendations or by vacuum concentration. If final volume falls below 32 μL after the concentration step, add water to bring the volume up to 32 μL. At this point, samples can be stored at −20° C. until needed or can be used immediately in the Enrichment protocol.

F. Target Enrichment

In some cases, the following target enrichment protocol can be performed: 1. Remove the Target Extension Buffer Mix (clear: TX1) and Target Probe Pool (e.g., S02007, 502008, 502009, 502010 or 502011) from −20° C. storage. 2. Thaw TX1 and Target Probe Pool at room temperature. Mix by vortexing, spin and place on ice. 3. Prepare a master mix by combining TX1 and Target Probe Pool in a 0.5 mL capped tube, according to the volumes shown in Table 6. 4. Add 17 μL of Target Enrichment Master Mix to 32 μL of pooled, ligated gDNA. Mix by vortexing. Spin down briefly. 5. Place the tubes in a pre-warmed thermal cycler programmed to run Program 3 (Probe Hybridization and Extension; see Table 3): 95° C. −5 min, 200 cycles (80° C. −10 sec, decrease 0.1° C. each cycle), hold at 60° C. (after a minimum of 12 hours at 60° C., open lid of thermal cycler, open tube caps and, without removing the tubes from the thermal cycler, add 1 μL of Target Extension Enzyme Mix (clear: TX2). Pipette mix up and down more than 5 times with a volume equal to or greater than 40 μL Reseal the tubes, close the thermal cycler lid and immediately continue to the next step of the program), 72° C. for 10 min, and hold at 4° C. 6. Remove the tubes from the thermal cycler, spin to collect condensation and place on ice. 7. Continue immediately with the Enriched Library Purification protocol.

TABLE 6
Target Enrichment Mix (volumes listed are for a single reaction)

| TARGET EXTENSION BUFFER MIX | TARGET PROBE POOL |
|---|---|
| (CLEAR: TX1) | (CLEAR: N/A) |
| 7 μL | 10 μL |

Step 5 can also be performed by Alternative Quick Hybridization and Extension to run the quick protocol to complete the target enrichment and library preparation in a single day. A larger amount of input gDNA can be used for the Quick Protocol. Lower input amounts can exhibit lower on-target reads and elevated duplication rates. Alternative step 5. Place the tubes in a pre-warmed thermal cycler programmed to run Program 3 (Probe Hybridization and Extension; see Table 3): 95° C. for 5 min, 200 cycles (80° C. for 10 sec, decrease 0.1° C. each cycle), hold at 60° C. (After a minimum of 2 hours at 60° C., open lid of thermal cycler, open tube caps and, without removing tubes from the thermal cycler, add 1 μL of Target Extension Enzyme Mix (clear: TX2). Pipette mix up and down more than 5 times with a volume equal to or greater than 40 μL. Reseal the tubes, close the thermal cycler lid and immediately continue to the next step of the program), 72° C. −10 min, 10 cycles of (95° C. for 20 sec, 60° C. for 30 sec, 72° C. for 30 sec) 72° C. for 10 min, hold at 4° C.

G. Enriched Library Purification

In some cases, the following enriched library purification protocol can be used: This procedure can involve two sequential bead purifications. 1. Retrieve the Agencourt RNAClean XP Beads from 4° C. and ensure they are at room temperature before using. Also remove the DNA Resuspension Buffer (DR1) from −20° C. and thaw at room temperature. 2. Resuspend the beads by inverting and tapping the tube. Ensure the beads are fully resuspended before adding to the sample. After resuspending, the beads do not need to be spun. (An excess of beads can be provided; therefore, it is not necessary to recover any trapped in the cap.) 3. Add 50 μL of room-temperature Nuclease-free Water (D1) to each Target Extension reaction. 4. Add 80 μL (0.8 volumes) room temperature bead suspension to each sample. Mix thoroughly by pipetting 10 times. 5. Incubate at room temperature for 5 minutes. 6. Transfer the tubes to the magnet plate and let stand 5 minutes to completely clear the solution of beads. 7. Remove only 160 μL of the binding buffer and discard it. Leaving some of the volume behind can minimize bead loss at this step. Note: The beads can stay on the walls of the tubes. Significant loss of beads at this stage can impact the amount of DNA carried into Library Amplification, so in some cases ensure beads are not removed with the binding buffer or the wash. 8. With the plate still on the magnet, add 180 μL of freshly prepared 85% ethanol and allow to stand for at least 30 seconds but no more than 1 minute. 9. Remove the ethanol wash using a pipette. 10. Air dry the beads on the magnet for a minimum of 10 minutes. Inspect each tube to check that all the ethanol has evaporated. All residual ethanol can be removed prior to continuing. 11. Remove the tubes from the magnet. 12. Add 100 μL of DNA Resuspension buffer (DR1) to the dried beads. Mix thoroughly by pipetting to ensure all the beads are resuspended. Incubate for 2 minutes at room temperature. 13. Transfer the tubes to the magnet and let stand for 3 minutes to completely clear the solution of beads. 14. Carefully remove 100 μL of the eluate to a new tube. 15. Add 80 μL (0.8 volumes) room temperature bead suspension to the 100 μL eluate. Mix thoroughly by pipetting 10 times. 16. Incubate at room temperature for 5 minutes. 17. Transfer the tubes to the magnet and let stand 5 minutes to completely clear the solution of beads. 18. Carefully remove only 160 μL of the binding buffer and discard it. Leaving some of the volume behind minimizes bead loss at this step. Note: The beads may stay on the walls of the tubes. Significant loss of beads at this stage can impact the amount of DNA carried into Library Amplification, so in some cases ensure beads are not removed with the binding buffer or the wash. 19. With the tubes still on the magnet, add 180 μL of freshly prepared 85% ethanol and allow to stand for at least 30 seconds but no more than 1 minute. 20. Remove and discard ethanol wash. 21. Air dry the beads on the magnet for at least 10 minutes at room temperature. In some cases, inspect each tube to ensure that all the ethanol has evaporated. All residual ethanol can be removed prior to continuing. 22. Remove the tubes from the magnet. 23. Add 25 μL of DNA Resuspension buffer (DR1) to the dried beads. Mix thoroughly by pipetting to ensure all the beads are resuspended. Incubate for 2 minutes at room temperature. 24. Transfer the tubes to the magnet and let stand for 3 minutes to completely clear the solution of beads. 25. Remove 24 μL of the eluate, ensuring as few beads as possible are carried over. 26. At this point, samples can be stored at −20° C. until needed or can be used immediately for qPCR (if being performed) or in the Library Amplification protocol.

H. qPCR (Optional)

When using compositions or kits provided herein, e.g., the Ovation Target Enrichment System for the first time with a new probe set or sample type, a qPCR assay can be performed in order to determine the number of cycles necessary for library amplification.

I. Library Amplification

In some cases, the following library amplification protocol can be used: 1. Remove the Library Amplification Buffer Mix (P1) Amplification Primer Mix (P2), Amplification Enzyme Mix (red: P3) and Amplification Enhancer Mix (P6) from −20° C. storage. 2. Spin down P3 and place on ice. 3. Thaw P1 and P2 at room temperature. Mix each by vortexing, spin and place on ice. 4. Prepare a master mix by combining P1, P2, P3, P6 and water in a 0.5 mL capped tube, according to the volumes shown in Table 7. 5. Add 78 μL of Library Amplification Master Mix to 22 μL of purified DNA from step G.25 above. 6. Mix well by pipetting. 7. Place the tubes in a pre-warmed thermal cycler programmed to run Program 4 (Library Amplification; see Table 3): 37° C. for 10 min, 95° C. for 3 min, 15-30 cycles* (95° C. for 30 sec, 62° C. for 15 sec, 72° C. for 20 sec; number of cycles determined by qPCR), 72° C. for 2 min, and hold at 4° C. 8. Remove the tubes from the thermal cycler, spin to collect condensation and place on ice. 9. Continue immediately with the Amplified Library Purification protocol.

TABLE 7
Amplification Master Mix (volumes listed are for a single reaction)

| AMPLIFI- | AMPLIFI- | AMPLIFI- | AMPLIFI- | |
|---|---|---|---|---|
| CATION | CATION | CATION | CATION | |
| BUFFER | PRIMER | ENHANCER | ENZYME | WATER |
| MIX (RED: | MIX (RED: | MIX (RED: | MIX (RED: | (GREEN: |
| P1 VER 4) | P2 VER 14) | P6) | P3 VER 2) | D1) |
| 20 μL | 8 μL | 4μL | 2 μL | 44 μL |

J. Amplified Library Purification

In some cases, the following amplified library purification protocol can be used: This procedure involves two sequential bead purifications. 1. Retrieve the Agencourt RNAClean XP Beads from 4° C. and ensure they are at room temperature before using. Also remove the DNA Resuspension Buffer (DR1) from −20° C. and thaw at room temperature. 2. Resuspend the beads by inverting and tapping the tube. Ensure the beads are fully resuspended before adding to the sample. After resuspending, the beads do not need to be spun. (An excess of beads can be provided; therefore, it is not necessary to recover any trapped in the cap.) 3. Add 80 μL (0.8 volumes) room temperature bead suspension to each sample. Mix thoroughly by pipetting 10 times. 4. Incubate at room temperature for 5 minutes. 5. Transfer the tubes to the magnet plate and let stand 5 minutes to completely clear the solution of beads. 6. Remove 160 μL of the binding buffer and discard it. Leaving some of the volume behind can minimize bead loss at this step. Note: The beads can stay on the walls of the tubes. Care may be taken to prevent beads from being removed with the binding buffer or the wash. 7. With the tubes still on the magnet, add 180 μL of freshly prepared 85% ethanol and allow to stand for at least 30 seconds but no more than 1 minute. 8. Remove and discard the ethanol wash. 9. Air dry the beads on the magnet for at least 10 minutes at room temperature. Inspect each tube to check that all the ethanol has evaporated. Remove ethanol prior to continuing. 10. Remove the tubes from the magnet. 11. Add 100 μL of DNA Resuspension buffer (DR1) to the dried beads. Mix thoroughly by pipetting to ensure all the beads are resuspended. Incubate for 2 minutes at room temperature. 12. Transfer the tubes to the magnet and let stand for 3 minutes to completely clear the solution of beads. 13. Remove 100 μL of the eluate to a new tube, attempting to transfer as few beads as possible. 14. Add 80 μL (0.8 volumes) room temperature bead suspension to the 100 μL eluate. Mix thoroughly by pipetting 10 times. 15. Incubate at room temperature for 5 minutes. 16. Transfer the tubes to the magnet and let stand 5 minutes to completely clear the solution of beads. 17. Remove only 160 μL of the binding buffer and discard it. Leaving some of the volume behind minimizes bead loss at this step. The beads can stay on the walls of the tubes. Care can be taken to prevent the beads from being removed with the binding buffer or the wash. 18. With the tubes still on the magnet, add 180 μL of freshly prepared 85% ethanol and allow to stand for at least 30 seconds but no more than 1 minute. 19. Remove and discard ethanol wash. 20. Air dry the beads on the magnet for at least 10 minutes at room temperature. Inspect each tube carefully to ensure that all the ethanol has evaporated. Remove residual ethanol prior to continuing. 21. Remove the tubes from the magnet. 22. Add 25 μL of DNA Resuspension buffer (DR1) to the dried beads. Mix thoroughly by pipetting to ensure all the beads are resuspended. Incubate for 2 minutes at room temperature. 23. Transfer the tubes to the magnet and let stand for 3 minutes to completely clear the solution of beads. 24. Carefully remove 24 μL of the eluate, ensuring as few beads as possible are carried over. 25. The library is now ready for quantification and sequencing and can be stored at −20° C. until needed.

K. Quantitative and Qualitative Assessment of the Library

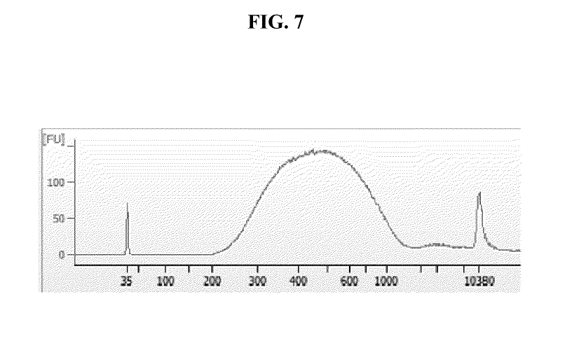

1. Run 0.5-1 μL of the sample on a Bioanalyzer High Sensitivity DNA Chip (Agilent Technologies). A typical fragment distribution is shown in FIG. 7, below. 2. Validate the library as described in the most recent Illumina user guides for DNA library construction, e.g., Genomic DNA Sample Prep Manual.

VI Guidelines for Optional qPCR

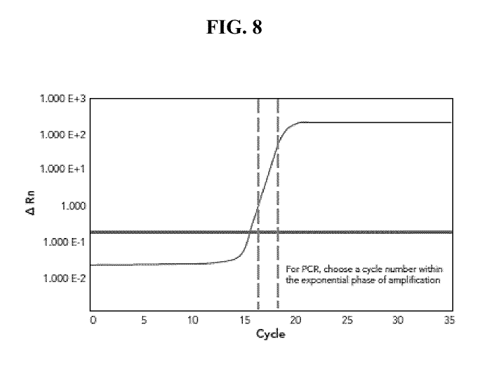

When using methods, compositions or kits as provided herein, e.g., the Ovation Target Enrichment System for the first time with a new probe set or sample type, a qPCR can be performed to assay after protocol step G.25 in order to determine the number of cycles necessary for subsequent library amplification. In some cases, the following protocol is used: 1. Remove the Library Amplification Buffer Mix (P1) Amplification Primer Mix (P2), Amplification Enzyme Mix (P3) and Amplification Enhancer Mix (P6) from −20° C. storage. 2. Spin down P3 and place on ice. 3. Thaw P1 and P2 at room temperature. Mix each by vortexing, spin and place on ice. 4. Prepare a master mix by combining P1, P2, P3, P6, water and dye (Eva Green or SYBR Green reagent) in a 0.5 mL capped tube, according to the volumes shown in Table 8. 5. Add 8 μL of qPCR Master Mix to 2 μL of purified library DNA (or water for the negative control) in a qPCR plate and mix well. 6. Perform qPCR using the following protocol: 37° C. for 10 min, 95° C. for 3 min, 35 cycles (95° C. for 30 sec, 62° C. for 15 sec, 72° C. for 20 sec). The cycle number used for subsequent library amplification can be within the exponential phase of the amplification plot (17 or 18 cycles in FIG. 8 as an example). In some cases, there is sufficient reagent included to perform a single test qPCR reaction for each of the multiplexed libraries.

TABLE 8
qPCR Master Mix (volumes listed are sufficient
for 2 reactions and a small overage)

| AMPLIFI- | AMPLIFI- | AMPLIFI- | AMPLIFI- | QPCR | |
|---|---|---|---|---|---|
| CATION | CATION | CATION | CATION | DYE AT | |
| BUFFER | PRIMER | ENHANCER | ENZYME | 1X FINAL | WATER |
| MIX (RED: | MIX (RED: | MIX (RED: | MIX (RED: | REACTION | (GREEN: |
| P1 VER 4) | P2 VER 14) | P6) | P3 VER 2) | CONC. | D1) |
| 5 μL | 2 μL | 1 μL | 0.5 μL | X | To total |
| | | | | | volume of |
| | | | | | 20 μL |

VII Sequencing Recommendations and Guidelines

Standard Illumina single end 50-150 base sequencing with a 14-base Indexing read can be used for most applications using methods, compositions and kits provided herein, e.g., the Ovation Target Enrichment System. For detecting translocations it can be beneficial to perform paired end sequencing. First, in some cases, trim the first 65 bases of the reverse read as this sequence is primarily probe derived and not useable for identifying variants. The second consideration with paired end sequencing is that the first 15 bases of all probe sequences are the same sequence and on the HiSeq platform can result in reverse read failures due to lack of diversity. This is not the case on the MiSeq which uses the color matrix generated in read 1 for all subsequent reads. However, the HiSeq recalculates the color matrix for the reverse read. To mitigate this problem, the user can specify a different lane of the flow cell that contains sufficient diversity to be used for lane alignment and error rate calculations. Please refer to Illumina's documented guidance on using a separate lane of sequence for lane alignment and error rate calculation or consult with Illumina technical support. The system uses 8-base barcodes and the same approach to multiplexing found in the standard Illumina method. The resulting libraries can be sequenced using the Illumina protocol for multiplex sequencing of 8-base barcodes, but with an additional 6 bases added to the index read for a total of 14 bases. The additional 6 bases can be used for true duplicate read determination using the Duplicate Marking Software Tool. The 8-base barcode sequences can be accessed through the barcodes link in the left hand menu of the Ovation Target Enrichment product page at www.nugen.com and may be entered into the sample sheet for the Illumina sequencing run prior to the analysis. The additional 6 random bases can be entered as ‘N’s.

VIII. Optional Duplicate Determination

The index reads can consist of eight bases for library identification followed immediately by six random bases. By increasing the indexing read length settings on the sequencer to 14 bases and using the NuGEN Technologies Duplicate Marking Software tool, you can be able to accurately identify PCR duplicates in the data set. The 8-base barcodes followed by NNNNNN can be entered into the sample sheet to enable proper multiplex library parsing. The Duplicate Marking tool and instructions for use can be obtained by contacting NuGEN Technical Support (techserv@nugen.com) or from the NuGEN website, www.nugen.com.

IX. Barcode Sequences and Guidelines for Multiplex Experiments

The Ovation Target Enrichment System 32-reaction kit can contain 32 different barcodes and the 96-reaction kit contains 96 different barcodes that can be used for multiplex sequencing to interrogate several independent libraries on a single lane of the Illumina NGS platforms. Barcode sequences and multiplex guidelines for adaptors used in Ovation Target Enrichment Systems can be found in Table 9. All barcode sequences can be separated by an edit distance of three. For further details on the barcode design strategy, please refer to Faircloth B C, Glenn T C (2012) Not All Sequence Tags Are Created Equal: Designing and Validating Sequence Identification Tags Robust to Indels. PLoS ONE 7(8): e42543. doi:10.1371/journal.pone.0042543, which is incorporated herein by reference in its entirety.

TABLE 9
The 32-reaction kit can contain the 32 barcodes shown in
plate positions A01-H04. The 96-reaction kit can
contain 96 barcodes in plate positions as shown. Barcodes
can be color-balanced insets of 8 by column on the plate.
(e.g., A01-H01 is a color-balanced set of barcodes.)

| PLATE | BARCODE | PLATE | BARCODE | PLATE | BARCODE |
|---|---|---|---|---|---|
| POSITION | SEQUENCE | POSITION | SEQUENCE | POSITION | SEQUENCE |
| A01 | CACGTCTA | A05 | ATGGCGAT | A09 | CAAGAAGC |
| B01 | AGCTAGTG | B05 | AACGCCTT | B09 | CTGATGAG |
| C01 | ACTATCGC | C05 | GTAAGGTG | C09 | CTCGAACA |
| D01 | GCGTATCA | D05 | TGTCGACT | D09 | TCGACAAG |
| E01 | ACTCTCCA | E05 | ACTCTGAG | E09 | AGTGCATC |
| F01 | CGTCCATT | F05 | GATGGAGT | F09 | TGGCTACA |
| G01 | AGCCGTAA | G05 | CTAGCTCA | G09 | GCACACAA |
| H01 | GAGTAGAG | H05 | CTGTACCA | H09 | GCATAGTC |
| A02 | ACGTCGTT | A06 | CCTGTCAA | A10 | AACACGCT |
| B02 | GTCCTGTT | B06 | GGTCGTAT | B10 | TTCACGGA |
| C02 | AGAAGCCT | C06 | CGCTGATA | C10 | TGCTGTGA |
| D02 | GAAGATCC | D06 | TAGCTTCC | D10 | CCTCGAAT |
| E02 | TAGCTGAG | E06 | CAAGTCGT | E10 | TGTGGCTT |
| F02 | ACGTCCAA | F06 | GTCTCATC | F10 | CTGTGGTA |
| G02 | CACACATC | G06 | ACCAAGCA | G10 | TCACTCGA |
| H02 | CGGATCAA | H06 | AGTCAGGT | H10 | ACTCCTAC |
| A03 | TCAGCCTT | A07 | TATCGCGA | A11 | CCACAACA |
| B03 | AAGGCTCT | B07 | TAGCAGGA | B11 | CCGCTTAA |
| C03 | TGTTCCGT | C07 | AGAAGGAC | C11 | GTGGTATG |
| D03 | GGAATGTC | D07 | TGAGCTGT | D11 | GGTGTACA |
| E03 | CATCCAAG | E07 | CAGAGTGA | E11 | TCTAGGAG |
| F03 | GTCAACAG | F07 | AGGTTCCT | F11 | TGGAAGCA |
| G03 | TCGCTATC | G07 | AGACCTTG | G11 | AACACCAC |
| H03 | AGCCTATC | H07 | CTTCCTTC | H11 | CATACGGA |
| A04 | TCGGATTC | A08 | CAGGTTCA | A12 | CTCTCAGA |
| B04 | CGGAGTAT | B08 | ACTGGTGT | B12 | AAGCTGGT |
| C04 | GAACCTTC | C08 | GGATTCAC | C12 | GTGTCCTT |
| D04 | AGAGGATG | D08 | CACGATTC | D12 | AGGTCTGT |
| E04 | ACGCTTCT | E08 | AGACATGC | E12 | CATTCGTC |
| F04 | CACAGGAA | F08 | GACACAGT | F12 | CTCACCAA |
| G04 | ACGAATCC | G08 | CCAGTTGA | G12 | GACTACGA |
| H04 | CCTTCCAT | H08 | CATGGATC | H12 | ATACGCAG |

X Other Considerations.

The custom kits can be available as 32 or 96 reaction size with the corresponding number of unique barcodes.

The kit can have a shelf life of 6 months from receipt.

A member of a Technical Services team can help to obtain the correct information for a custom design. RefSeq IDs or genomic coordinates will generally suffice.

If the targets are provided in the appropriate format, it can take no more than a couple of days for the design to be complete. For non-human sequence this process can take longer in order for the correct genome sequence to be identified and added to the design pipeline. The estimated turnaround time can be 3-4 weeks between obtaining customer approval on a finalized design and shipping the custom kit.

The genomic coordinates and 50 bases of sequence information are also provided that includes the probe.

Covaris focused acoustic shearing can be used to shear the DNA, and other methods (e.g., fragmentase) can work.

The Ovation Target Enrichment System features a simple add and incubate workflow, so it can be automated.

FFPE material can works well in the system and can work best with a probe design optimized to accommodate the smaller expected fragment sizes of FFPE genomic DNA. Because the DNA is modified and degraded, starting with a larger amount of input material (more than 100 ng) can be preferred.

In some cases, cell-free DNA can require an optimized probe design that addresses smaller fragments, similar to FFPE material. If there is sufficient plasma DNA as input, this may be possible.

In some cases, genomic DNA from a single cell can require a preamplification step prior to the library prep and enrichment.

Probes can be designed for regions that target the differences between genes and pseudogenes.

In design, probes can be placed outside the target region. However, for larger target regions a probe can be tiled within the target region in order to make sure that longer exons are accurately represented.

Reads up to 150 bp can work. Longer reads can be performed and can require a larger average fragment size and the read may extend into the probe.

Single end reads can be sufficient and advised for many applications. Paired end sequencing can be performed to look specifically for rearrangements like inversions and translocations.

Due to the probe extension technology employed by the target enrichment system, the first 65 bases of reverse read sequence are probe-derived and can be trimmed prior to alignment.

### Example 6

### Enrichment cDNA from 100 ng of UHR RNA

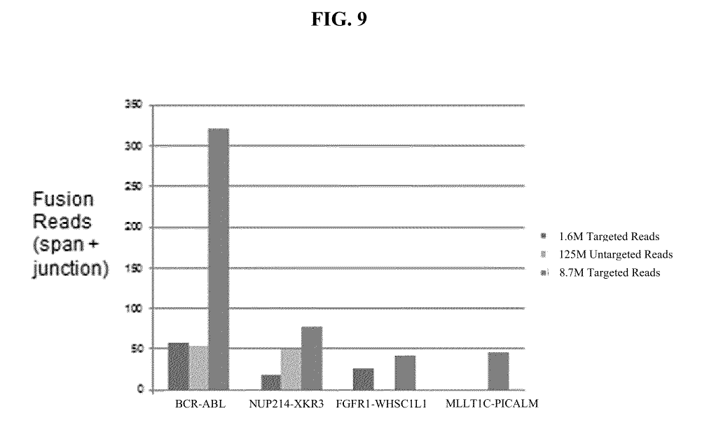

100 ng of universal human reference (UHR) RNA was used as input into the Ovation target enrichment cDNA module and libraries were prepared with the Gene Fusion Panel of probes following the manufacturers protocol. Sequencing libraries were quantitated by Bioanalyzer (Agilent Technologies, USA) and Kapa Library Quantification Kit (KAPA Biosytems, USA). Libraries were sequenced on the Illumina Hiseq as paired end reads, 100 bases in length. Resulting sequences were analyzed for the presence of gene fusions with SOAPFuse v1.22 software using default settings (//soap.genomics.org.cn/soapfuse.html). Standard RNAseq data were obtained for comparison at //sra.dnanexus.com/studies/SRP028705. The targeted reads and untargeted reads are shown in FIG. 9.

### Example 7

### Selective Target Enrichment Using a Priming Reaction to Append Forward and Reverse Adaptors

In this example, both forward and reverse adaptors are appended to a nucleic acid sequence by priming reactions. As depicted in FIG. 10, nucleic acids are fragmented to generate a plurality of fragmented nucleic acids 1001. The nucleic acid fragments are denatured and adaptor 1 is appended to the nucleic acid fragments by annealing primer A with adaptor 1 at the 5′ end to the nucleic acid fragments 1003, 1005. Primer A can anneal to either strand of the nucleic acid fragments 1003, 1005. Primer A is extended with a polymerase to generate an oligonucleotide extension product 1003, 1005. After extension, primer B is added 1007, 1009 that anneals to a sequence on the oligonucleotide extension product. Primer B has an adaptor 2 sequence at the 5′ end and a sequence that anneals to the oligonucleotide extension product 1007, 1009. Primer B is extended with a polymerase 1007, 1009 to generate a second oligonucleotide extension product with the complement of adaptor 1 at a first end, the target sequence of interest, and adaptor 2 at a second end. Primer A may be random when primer B is target specific or primer A may be target specific when primer B is random. The second oligonucleotide extension product is then amplified using primers that anneal to the complement of adaptors 1 and 2 1011, 1013.

### Example 8

### Selective Target Enrichment Using a Reverse Protocol to Append Forward and Reverse Adaptors

As depicted in FIG. 11, target-specific sequences can be enriched using a protocol that appends a first adaptor to a nucleic acid fragment by annealing and extending a primer and then ligating a second adaptor to the opposite end of the nucleic acid fragment. Nucleic acids are fragmented to generate fragmented nucleic acid sequences 1101. The first adaptor is appended to the nucleic acids by annealing a primer comprising a target-specific portion at the 3′ end and adaptor 1 at the 5′ end 1103. The target-specific portion of the primer will anneal to a target-specific sequence in the nucleic acid fragments 1103. The primer is extended by addition of a polymerase 1103 generating an oligonucleotide extension product 1103 comprising a sequence complementary to the target-specific sequence and adaptor 1 at a first end 1103. Double-stranded adaptor 2 is ligated to the double-stranded blunt-end extension product 1105. The double-stranded extension product is denatured and a primer comprising adaptor 2 sequence is annealed and extended 1107. The target library is amplified with primers that are specific for adaptor 1 and adaptor 2 1109.

### Example 9

### Selective Target Enrichment Using Transposon-Tagging

As depicted in FIG. 12, target-specific sequences can be enriched using transposon-tagging. In this example, a transposome complex containing a transposase enzyme and a transposon sequence are combined with a nucleic acid 1201. The transposome complex randomly binds to the nucleic acid and simultaneously fragments the nucleic acid and tags the nucleic acid with the transposon sequence 1203 (TS1 and TS2). The known transposon sequence then acts as a primer-binding site for a primer that has an adaptor 1 sequence at the 5′ end and a sequence complementary to the transposon sequence at the 3′ end 1205. The primer is annealed to the nucleic acid fragment and extended with a polymerase 1205 generating an oligonucleotide extension product with a sequence complementary to the nucleic acid fragment and adaptor 1 at a first end 1207. A target-specific primer that contains a sequence that anneals to the complement of a target nucleic acid sequence at the 3′ end and an adaptor 2 at the 5′ end is then annealed to the oligonucleotide extension product 1207 and extended by a polymerase to generate a second oligonucleotide extension product with the complement of adaptor 1 at a first end (C1), the target nucleic acid sequence and the second adaptor at a second end. The target nucleic acid sequence is then amplified with primers that anneal to the complement of adaptor 1 1209 and the complement of adaptor 2 1211 thereby generating a double-stranded nucleic acid molecule with adaptor 1 at one end and adaptor 2 at the other end 1213.

### Example 10

### Selective Target Enrichment Using Transposon-Tagging

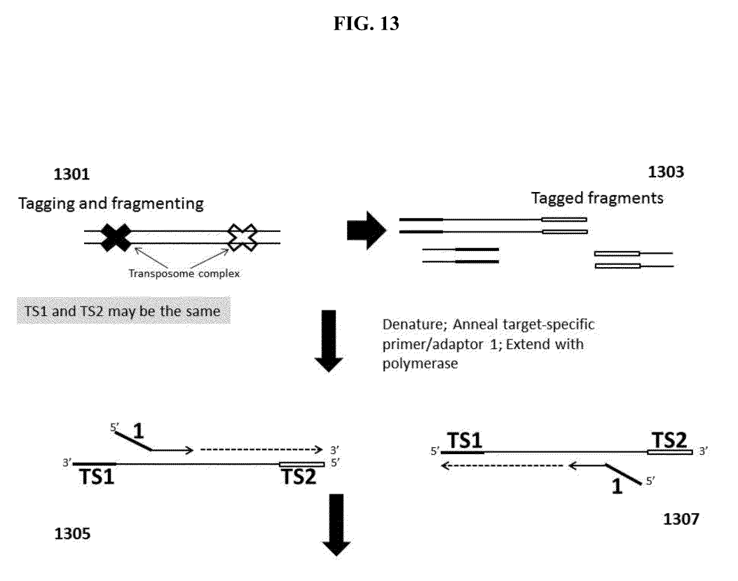

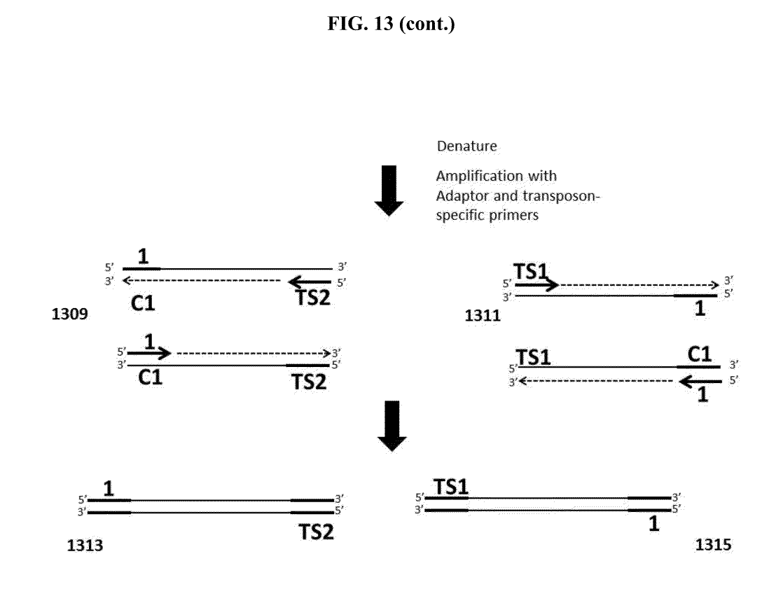

As depicted in FIG. 13, target-specific sequences can be enriched using transposon-tagging. In this example, a transposome complex containing a transposase enzyme and a transposon sequence are combined with a nucleic acid 1301. The transposome complex randomly binds to the nucleic acid and simultaneously fragments the nucleic acid and tags the nucleic acid with the transposon sequence 1303 (TS1 and TS2). The tagged fragments are denatured and a primer that contains a sequence that anneals to the complement of a target nucleic acid sequence at the 3′ end and an adaptor 1 sequence at the 5′ end is annealed to a nucleic acid fragment 1305, 1307 and extended by a polymerase to generate an oligonucleotide extension product. The double-stranded oligonucleotide extension product is then denatured and the target nucleic acid sequence is amplified using primers that anneal to the complement of adaptor 1 and the transposon sequence 1309, 1311 thereby generating a double-stranded nucleic acid molecule with adaptor 1 at one end and the transposon sequence at the other end 1313, 1315.

### Example 11

### Selective Target Enrichment Using a Priming Reaction to Append Forward and Reverse Adaptors

As depicted in FIG. 14, both forward and reverse adaptors can be appended to a nucleic acid sequence by priming reactions. Double-stranded nucleic acids containing a nucleic acid sequence of interest are denatured to produce single-stranded nucleic acid molecules. Primers containing random sequence (RS) at the 3′ end and adaptor 1 sequence (A1) at the 5′ end are annealed to the single-stranded nucleic acid molecules 1401. The random primers are composite primers (e.g., the random sequence includes DNA and adaptor 1 sequence includes RNA). The primers are extended with a polymerase to generate a first oligonucleotide extension product 1403. The first oligonucleotide extension product is denatured and target-specific primers (TS) are annealed to the first oligonucleotide extension product 1405, wherein the target-specific primers contain a 3′ end that specifically anneals to the nucleic acid sequence of interest within the first oligonucleotide extension product and a 5′ end that contains adaptor 2 sequence (A2). The target-specific primers are extended with a polymerase to generate a second oligonucleotide extension product forming an RNA/DNA heteroduplex between adaptor 1 (RNA) and the complement of adaptor 1 (DNA) 1407. The adaptor 1 sequence is digested with RNase H 1409 to generate a pool of tagged nucleic acid molecules containing the nucleic acid sequence of interest 1411. The nucleic acid sequence of interest is then amplified and enriched with primers (A1 and A2) that bind to the complement of the adaptor 2 sequence (A2′) and the complement of the adaptor 1 sequence (A1′) 1411, 1413.

1. A method for enriching for a nucleic acid sequence of interest in a sample comprising nucleic acids, the method comprising:
a) annealing an oligonucleotide to the nucleic acid sequence of interest in a nucleic acid fragment, wherein the oligonucleotide comprises a 3′ portion complementary to the nucleic acid sequence of interest and a 5′ portion comprising a first adaptor sequence;
b) extending the oligonucleotide annealed to the nucleic acid sequence of interest in the nucleic acid fragment with a polymerase, thereby generating an oligonucleotide extension product comprising sequence complementary to the nucleic acid sequence of interest and the first adaptor sequence at a first end;
c) ligating a sequence complementary to a second adaptor sequence to the oligonucleotide extension product, thereby generating an oligonucleotide extension product comprising the first adaptor sequence at the first end, the sequence complementary to the nucleic acid sequence of interest, and a sequence complementary to the second adaptor sequence at a second end; and
d) amplifying the nucleic acid sequence of interest using a first primer that anneals to the complement of the first adaptor sequence and a second primer that anneals to the complement of the second adaptor sequence to enrich for the nucleic acid sequence of interest, thereby generating an enriched nucleic acid sequence of interest.

2. The method of claim 1, further comprising, before step a), fragmenting a nucleic acid in the sample, thereby generating the nucleic acid fragment of step a) comprising the sequence of interest.

3. The method of claim 1, wherein the nucleic acid fragment is double-stranded prior to step a).

4. The method of claim 1, wherein the nucleic acid fragment comprises deoxyribonucleic acid (DNA).

5. The method of claim 4, wherein the DNA is genomic DNA.

6. The method of claim 4, wherein the DNA is cDNA.

7. The method of claim 3, further comprising denaturing the double-stranded nucleic acid fragment prior to step b), wherein the nucleic acid fragment comprising the nucleic acid sequence of interest in step b) is single-stranded prior to annealing to the oligonucleotide.

8. The method of claim 1, wherein the 3′ portion of the oligonucleotide comprises a random sequence.

9. The method of claim 1, wherein the 3′ portion of the oligonucleotide comprises a sequence designed to anneal to the nucleic acid sequence of interest.

10. The method of claim 1, wherein ligating the sequence complementary to the second adaptor sequence comprises ligating the sequence complementary to the second adaptor sequence to a 3′ end of the oligonucleotide extension product.

11. The method of claim 10, wherein the second adaptor sequence is within a strand of a double-stranded adaptor.

12. The method of claim 1, wherein the first adaptor sequence and the second adaptor sequence are distinct.

13. The method of claim 1, wherein the first adaptor sequence and/or the second adaptor sequence comprise barcode sequence.

14. The method of claim 1, wherein the first adaptor sequence and/or the second adaptor sequence comprise sequence that can anneal to a sequencing primer, or complement of a sequence that can anneal to a sequencing primer.

15. The method of claim 1, wherein the polymerase is a DNA polymerase.

16. The method of claim 1, further comprising:
(i) annealing a plurality of oligonucleotides to different nucleic acid sequences of interest in nucleic acid fragments, wherein each of the plurality of oligonucleotides comprises a 3′ portion complementary to a nucleic acid sequence of interest among the nucleic acid sequences of interest and a 5′ portion comprising a first adaptor sequence;
(ii) extending the plurality of oligonucleotides annealed to the different nucleic acid sequences of interest in the nucleic acid fragments with a polymerase, thereby generating a plurality of oligonucleotide extension products comprising sequence complementary to the nucleic acid sequences of interest and the first adaptor sequence at a first end;
(iii) ligating a sequence complementary to a second adaptor sequence to the plurality of oligonucleotide extension products, thereby generating a plurality of oligonucleotide extension products comprising the first adaptor sequence at the first end, the sequence complementary to the nucleic acid sequence of interest, and a sequence complementary to the second adaptor sequence at a second end; and
(iv) amplifying the nucleic acid sequences of interest using a first primer that anneals to the complement of the first adaptor sequence and a second primer that anneals to the complement of the second adaptor sequence to enrich for the nucleic acid sequences of interest, thereby generating enriched nucleic acid sequences of interest.

17. The method of claim 1, wherein the amplifying in step d) comprises a polymerase chain reaction.

18. The method of claim 1, further comprising enriching a plurality of nucleic acid sequences of interest using the steps of claim 1.

19. The method of claim 18, further comprising sequencing the plurality of enriched nucleic acid sequences of interest.

20. The method of claim 1, wherein the nucleic acid sequence of interest comprises a gene.

21. The method of claim 20, wherein the gene is a cancer gene.

22. The method of claim 1, further comprising sequencing the enriched nucleic acid sequence of interest.

23. The method of claim 22, wherein the sequencing comprises use of a reversible-dye terminator.

24.-88. (canceled)
